(19) United States
(12) Patent Application Publication
(10) Pub. No.: US 2022/0260565 A1
(43) Pub. Date: Aug. 18, 2022

(54) METHODS OF TREATING AND DIAGNOSING INFLAMMATORY BOWEL DISEASE

(71) Applicant: CEDARS-SINAI MEDICAL CENTER, Los Angeles, CA (US)

(72) Inventors: David Underhill, Tarzana, CA (US); Dermot McGovern, Los Angeles, CA (US); Jose Limon, Los Angeles, CA (US); Gil Y. Melmed, Los Angeles, CA (US)

(73) Assignee: CEDARS-SINAI MEDICAL CENTER, Los Angeles, CA (US)

(21) Appl. No.: 17/279,259

(22) PCT Filed: Sep. 19, 2019
(86) PCT No.: PCT/US19/51995

§ 371 Date: Mar. 24, 2021

Related U.S. Application Data
(60) Provisional application No. 62/745,785, filed on Oct. 15, 2018.

(51) Int. Cl. G01N 33/569; A61K 31/4196; A61K 31/7048

(52) U.S. Cl. CPC G01N 33/56961; A61K 31/4196; A61K 2039/505; C07K 2317/24; A61K 31/7048

(57) ABSTRACT

Described herein are methods of treatment of an inflammatory condition related to fungal immunity. The present disclosure relates to methods and systems for identifying patients suitable for treatment with active agents, as described herein. Further, described herein are various compositions for treating and identifying a subject in need of an active agent for treatment.

### CROSS-REFERENCE TO RELATED APPLICATIONS

This application is a National Phase of International Application No. PCT/US2019/051995, filed Sep. 19, 2019, which designated the U.S. and that International Application was published under PCT Article 21(2) in English, which claims priority under 35 U.S.C. § 119(e) to U.S. provisional patent application No. 62/745,785 filed Oct. 15, 2018, the entirety of which is hereby incorporated by reference.

### STATEMENT REGARDING FEDERALLY SPONSORED RESEARCH OR DEVELOPMENT

This invention was made with government support under Grant Nos. DK093426, DK046763 and TR001881 awarded by National Institutes of Health. The government has certain rights in the invention.

### FIELD OF INVENTION

This inventive subject matter relates to methods treating and diagnosing inflammatory disease related to fungal organisms in a subject in need thereof.

### BACKGROUND

Inflammatory bowel disease (IBD) is a significant cause of morbidity in the United States, with roughly three million adults affected by Crohn's disease (CD) or ulcerative colitis (UC), the two most common forms of IBD. Both CD and UC are chronic, relapsing inflammatory disorders of the gastrointestinal tract. IBD susceptibility and severity are influenced by a combination of genetics, microbiota, and environment. The intestinal microbiota in patients with IBD includes fungi (mycobiota).

There has been progress defining genetic, intestinal microbiome, host pathway, and environmental roles in disease. However, there is no cure for IBD; treatments have largely focused on targeting aberrantly active immune pathways, and they are effective in only a minority of patients. A major goal in IBD research and care is to improve outcomes by better matching disease treatment with patients' specific underlying mechanisms of disease.

There is a need for a better understanding of immune responses to intestinal fungi in the context of IBD, in order to best select and treat patients with IBD for which an antimycotic therapy may be useful to prevent or ameliorate inflammatory disease or symptoms of inflammatory disease.

### SUMMARY OF INVENTION

Various embodiments of the present invention provide for a method of treating an inflammatory disease or condition in a subject in need thereof, comprising: administering an anti-fungal therapy to the subject in need thereof, wherein the subject in need thereof has a fungus or an elevated level of a fungus as compared to a control level of the fungus in the gastrointestinal system.

Various embodiments of the present invention provide for method of manipulating the microbiome of a subject having an inflammatory disease or condition, comprising: administering an anti-fungal therapy to the subject, wherein the subject in need thereof has a fungus or an elevated level of a fungus as compared to a control level of the fungus in the gastrointestinal system.

Various embodiments of the present invention provide for a method of inhibiting or reducing fungal growth in a subject having an inflammatory disease or condition, comprising: administering an anti-fungal therapy to the subject in need thereof, wherein the subject in need thereof has a fungus or an elevated level of a fungus as compared to a control level of the fungus in the gastrointestinal system.

In various embodiments, the subject in need thereof can have a c-type lectin domain containing 7A (CLEC7A) gene risk variant, a caspase recruitment domain family member 9 (CARD9) gene risk variant or both. In various embodiments, the subject in need thereof can have a caspase recruitment domain family member 9 (CARD9) gene risk variant A in rs4077515 resulting in an amino acid substitution S12N. In various embodiments, the subject in need thereof can have an elevated level of a serological marker, as compared to a control level of the serological marker, said serological marker selected from the group consisting of anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globosa antibody, anti-Cladosporium spp. antibody, and a combination thereof.

In various embodiments, the fungus can be Malassezia, Cladosporium or both.

In various embodiments, the inflammatory disease can be inflammatory bowel disease. In various embodiments, the inflammatory disease can be ulcerative colitis or Crohn's disease.

In various embodiments, the anti-fungal therapy can comprise posaconazole. In various embodiments, the anti-fungal therapy can comprise a fungicide agent or a fungistatic agent. In various embodiments, the anti-fungal therapy can comprise an antimycotic agent. In various embodiments, the antimycotic agent can comprise a polyene, an azole, an echinocandin, an flucytosine, an allylamine, a tolnaftate, or griseofulvin. In various embodiments, the azole can comprise triazole, imidazole, clotrimazole, ketoconazole, itraconazole, terconazole, oxiconazole, miconazole, econazole, tioconazole, voriconazole, fluconazole, isavuconazole, itraconazole, pramiconazole, ravuconazole, or posaconazole. In various embodiments, the polyene can comprise amphotericin B, nystatin, or natamycin. In various embodiments, the echinocandin can comprise caspofungin, anidulafungin, or micafungin. In various embodiments, the allylamine can comprise naftifine or terbinafine.

Various embodiments of the present invention provide for a method of determining a level of a fungus, a fungal antigen, an anti-fungal antibody, or an enzyme of a fungus, in a subject in need thereof, comprising obtaining a biological sample from the subject; subjecting the biological sample to an assay suitable to detect the fungus, the fungal antigen the anti-fungal antibody, or the enzyme of a fungus, in the biological sample; and measuring the level of the fungus, the fungal antigen, the anti-fungal antibody, or the enzyme of the fungus, to determine the level of the fungus, the fungal antigen, the anti-fungal antibody, or the enzyme of the fungus.

Various embodiments of the present invention provide for a method of diagnosing an inflammatory disease or condition in a subject, comprising: determining a level of a fungus, a fungal antigen, an anti-fungal antibody, or an enzyme of a fungus, in a subject in need thereof accordingly to a method of the present invention; and diagnosing the subject with the inflammatory disease or condition when an elevated level of the fungus, the fungal antigen, the anti-fungal antibody, or the enzyme of a fungus as compared to each respective control level, is measured in the biological sample.

In various embodiments, detecting the presence of the fungal antigen can comprise contacting an antibody capable specifically binding to the fungal antigen to the biological sample to form a binding complex, and detecting the presence of the binding complex.

In various embodiments, detecting the presence of anti-fungal antibody comprises contacting an antibody capable specifically binding to the anti-fungal antibody to form a binding complex, and detecting the presence of the binding complex.

In various embodiments, detecting the presence of the enzyme of a fungus can comprise contacting an antibody capable specifically binding to the enzyme to form a binding complex, and detecting the presence of the binding complex.

In various embodiments, the biological sample can be selected from the group consisting of blood plasma, blood serum, stool, intestinal aspirate, intestinal tissue sample, intestinal mucosa, intestinal mucus and combinations thereof.

In various embodiments, the fungus can be Malassezia, Cladosporium, or both; the fungal antigen can be from Malassezia, Cladosporium, or both; the anti-fungal antibody can be an antibody capable of specifically binding to Malassezia, Cladosporium, or both, or specifically binding to an antigen of Malassezia, Cladosporium, or both; and the enzyme can be a lipase.

In various embodiments, the enzyme can be Malassezia lipase 1 or Cladosporium lipase 1.

In various embodiments, the subject in need thereof can have a c-type lectin domain containing 7A (CLEC7A) gene risk variant, a caspase recruitment domain family member 9 (CARD9) gene risk variant or both.

In various embodiments, the subject in need thereof can have a caspase recruitment domain family member 9 (CARD9) gene risk variant A in rs4077515 resulting in an amino acid substitution S12N.

In various embodiments, wherein the subject can have an elevated level of a serological marker, as compared to a control level of the serological marker, said serological marker selected from the group consisting of anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globosa antibody, anti-Cladosporium spp. antibody, and a combination thereof.

In various embodiments, the subject can have an elevated level of a serological marker, as compared to a control level of the serological marker, said serological marker is an enzyme of a fungus.

In various embodiments, wherein the enzyme is Malassezia lipase 1, or Cladosporium lipase 1.

### BRIEF DESCRIPTION OF THE FIGURES

Exemplary embodiments are illustrated in referenced figures. It is intended that the embodiments and figures disclosed herein are to be considered illustrative rather than restrictive.

FIGS. 1A-1F depict, in accordance with various embodiments herein, the mycobiome characterization. FIG. 1A) Sequences from non-diseased control and Crohn's disease (CD) sigmoid colon and cecum were identified to the genus level, and the relative representation of each genus in the datasets is illustrated. “Others” include 67 relatively rare genera, and “Unmapped” indicates the fraction of the sequences that could not be convincingly identified as belonging to specific genera (defined as ≥97% identity to a known reference sequence). FIG. 1B) The data are shown broken down by individual patient (x-axis), showing the relative abundance of sequences detected in each sample. FIG. 1C) MaAsLin analysis reveals that CD is associated with a decrease in ascomycetes and an increase in basidiomycetes compared to non-diseased controls (HC). Notched box plots show individual arcsine square-root transformed relative abundance's median and confidence intervals as well as the first and third quartiles. The strength of the difference in the normal samples is noted by the brightness (lower in CD—green (*), higher in CD—red (\hat{ })). FIG. 1D) Specific genera and their associations with CD (all patients) are illustrated. FIG. 1E) Analysis as in FIG. 1D but restricted to patients with Ileocolonic Crohn's disease. FIG. 1F) Presence or absence of the CD-associated variant of CARD9 was assessed for association with fungal genera. Fungal genera approaching or exceeding statistical significance (Bonferroni-corrected 6.7×10⁻⁴) are listed. FIG. 1G) Serum samples were selected from a biobank of CD patient sera previously characterized as “High” or “Low” for ASCA IgG or IgA (n=18-22 per group) and screened by flow cytometry for IgA and IgG reactivity against Malassezia restricta. (Mann-Whitney U Test) Notches indicate 95% confidence interval and whiskers extend no further than 1.5 times IQR from the hinge.

FIGS. 2A-2N depict, in accordance with various embodiments herein, M. restricta exacerbates colitis in mice. FIGS. 2A and 2B) Colon length upon termination of experiment in which mice were gavaged with the indicated live yeast and exposed to DSS in their drinking water for 7 days and then without DSS for 5 days (n=5/group). FIG. 2C) Disease activity over duration of experiment. FIG. 2D) Fecal lipocalin-2 levels were measured by ELISA on day 6. FIGS. 2E and 2F) Representative H&E-stained colon sections of an experiment terminated on day 7 (FIG. 2E) and histological assessment of disease severity (FIG. 2F). FIGS. 2G and 2H) Percentage of IL-17A- and IFN-γ-producing colonic Lamina propria CD4+ T cells was determined upon sacrifice (day 12). FIGS. 2I and 2J) Colon length upon termination of experiment in which fungal-free altered Schaedler flora (ASF) mice were gavaged with live yeast and exposed to DSS in their drinking water. FIG. 2K) Disease activity over duration of experiment. FIG. 2L) Fecal lipocalin-2 levels were measured by ELISA on day 5. FIGS. 2M and 2N) Percentage of IL-17A- and IFN-γ-producing colonic Lamina propria CD4+ T cells was determined upon sacrifice. (*, p<0.05; **, p<0.01; ***, p<0.001; one-way ANOVA with Tukey's multiple comparison test). Each dot represents an individual mouse. Data are representative of three independent experiments.

FIGS. 3A-3I depict, in accordance with an embodiment herein, immunostimulatory activity of M. restricta. FIG. 3A) C. albicans, S. cerevisiae, and M. restricta were grown in liquid culture and imaged by differential interference contrast microscopy. Diameters (n>150 each) were measured using ImageJ. FIGS. 3B-3F) M. restricta, C. albicans or S. cerevisiae were fixed (killed) in paraformaldehyde, and phagocytes were exposed to killed yeasts at the indicated multiplicities of infection (MOI), or to E. coli lipopolysaccharide (LPS, 100 ng/ml) for 24 hours. Cytokines in culture supernatants were measured by ELISA. FIG. 3B) TNF-α in culture supernatants of human dendritic cells. FIG. 3C) TNF-α and IL-6 in culture supernatants of mouse bone marrow-derived dendritic cells. FIG. 3D) TNF-α and IL-6 in culture supernatants of mouse bone marrow-derived macrophages. FIG. 3E) TNF-α in culture supernatants of mouse bone marrow-derived dendritic cells from wild-type (WT) or Card9−/− mice. FIG. 3F) Expression of CD86 by stimulated bone marrow-derived dendritic cells was assessed by flow cytometry. FIGS. 3G and 3H) Mouse bone marrow-derived dendritic cells were stimulated with the indicated fungi in the presence of anti-CD3ε antibodies and co-cultured with naïve CD4+ T cells. Production of IL-17A and IFN-γ by CD4+ cells was assessed by flow cytometry. FIG. 3I) Human peripheral blood-derived dendritic cells were prepared from non-diseased donors determined to be homozygous for the S12N CARD9 A (risk) or G (protective) alleles. Cells were stimulated (MOI 10) with the indicated yeasts or LPS (100 ng/ml) for 24 hours and production of TNF-α (n=9-11) and IL-8 (n=6-15) were measured. Each dot is an individual patient (measured in duplicate), and the boxes indicate means and standard deviations. (*, p<0.05; **, p<0.01; ***, p<0.001; one-way ANOVA with Tukey's multiple comparison test). Data in A-H are representative of three independent experiments.

FIGS. 4A-4E depict, in accordance with an embodiment herein, in vivo effects of M. restricta require CARD9. FIGS. 4A and 4B) Colon length upon termination of experiment in which Card9−/− mice or wild type littermates were gavaged with the indicated live yeast and exposed to DSS in their drinking water for 7 days and then without DSS for 5 days (n=6-7/group). FIGS. 4C and 4D) Disease activity over duration of experiment. FIG. 4E) Fecal lipocalin-2 levels were measured by ELISA on day 6. (*, p<0.05; **, p<0.01; *** p<0.001; one-way ANOVA with Tukey's multiple comparison test). Each dot represents an individual mouse. Data are representative of at least two independent experiments.

FIGS. 5A-5D depict, in accordance with an embodiment herein, fungal ITS sequencing characterization. FIG. 5A), Rarefaction curve of observed OTU richness in pooled mucosal samples. The curve depicts the number of OTUs observed at different sampling depths where the X axis is the number of ITS1 reads and the Y axis is the number of OTUs observed. After around 4000 reads the discovery of new OTUs tapers off FIG. 5B) Box-and-whisker plot illustrating the mapping efficiency in each of the sample groups. Whiskers represent maximum and minimum values. Samples in each of the groups were identified with similar efficiencies. FIG. 5C) A two-dimensional metric multidimensional-scaling (MDS) analysis approach was used to compare fungal compositions of paired sigmoid colon and cecum samples recovered from 28 patients. Paired samples are connected by lines. FIG. 5D) Sequences from non-diseased control and Crohn's disease sigmoid colon and cecum were identified to the phylum level, and the relative representation of each phylum in the datasets is illustrated. “Unmapped” indicates the fraction of the sequences that could not be convincingly identified (defined as ≥97% identity to a known reference sequence).

FIGS. 6A-6C depict, in accordance with an embodiment herein, fungal distribution in Crohn's Disease. FIG. 6A) MaAsLin analysis reveals that CD is associated with increased Aureobasidium and decreased Fusarium. Candida and Pichia are not different in CD. Notched box plots show individual arcsine square-root transformed relative abundance's median and confidence intervals as well as the first and third quartiles. The strength of the difference in the normal samples is noted by the brightness (lower in CD—green (*), higher in CD—red (\hat{ })). FIG. 6B) Relative decreases in detection of Trichoderma and Fusarium are noted when restricting analysis to patients with colonic Crohn's disease. FIG. 6C) Box-and-whisker plot showing relative abundance of Malassezia and Pichia in sigmoid colon CD samples (all patients, left, and patients with ileocolonic CD, right) according to the CARD9 S12N SNP genotype. (**, p<0.01; one-way ANOVA with Tukey's multiple comparison test).

FIGS. 7A-7C depict, in accordance with an embodiment herein, colitis in Malassezia- and Candida-treated mice. FIG. 7A) Experimental setup for oral gavage of mice with fungi and induction of colitis with DSS. FIG. 7B) Total number of IL-17A- and IFN-γ-producing colonic Lamina propria CD4+ T cells was determined upon sacrifice. FIG. 7C) Percentage of activated (CD44+) CD4+ T cells in the colonic Lamina propria and the mesenteric lymph nodes was determined upon sacrifice. (*, p<0.05; ***, p<0.001; one-way ANOVA with Tukey's multiple comparison test). Each dot represents an individual mouse. Data are representative of three independent experiments.

FIGS. 8A-8F depict, in accordance with an embodiment herein, colitis in M. restricta and S. cerevisiae-treated mice. FIGS. 8A and 8B) Colon length upon termination of experiment in which mice were gavaged with live yeast and exposed to DSS in their drinking water (n=5/group). Each dot represents an individual mouse. FIG. 8C) Disease activity over duration of experiment. FIGS. 8D and 8E) Colonic Lamina propria T cells were restimulated, and production of IL-17A and IFN-γ was measured by flow cytometry.

FIG. 8F) TNF-α and IL-6 mRNA levels in colon tissue was assessed by quantitative real-time PCR. (*, p<0.05; ** p<0.01; ***, p<0.001; one-way ANOVA with Tukey's multiple comparison test). Each dot represents an individual mouse. Data are representative of three independent experiments.

FIGS. 9A-9B depict, in accordance with an embodiment herein, fungal-free altered Schaedler flora mouse model. FIG. 9A) Detection of bacterial and fungal rDNA by quantitative PCR in fecal specimens of specific pathogen-free (SPF), germ-free (GF), altered Schaedler flora-colonized (ASF) mice, and ASF mice gavaged with M. restricta (n=5/group). FIG. 9B) Levels of the 8 ASF bacteria were assessed by quantitative PCR of 16 s rDNA before and after exposure to M. restricta. Each column is a different mouse as labeled.

FIG. 10 depicts, in accordance with an embodiment herein, the response of CARD9 S12N human cells to fungi. Human peripheral blood-derived dendritic cells were prepared from non-diseased donors determined to be homozygous for the S12N CARD9 A (risk) or G (protective) alleles (n=9-11). Cells were stimulated with the indicated yeasts (MOI 10, fixed in paraformaldehyde) or LPS (100 ng/ml) for 24 hours and production of cytokines was measured in the supernatants. IL-1β, IL-10, and IL-6 were measured in the supernatants. Each dot is an individual patient (measured in duplicate). The median and first and third quartiles are shown, and whiskers extend no further than 1.5 times IQR from the hinge. No responses were found to be significantly different between genotypes (one-way ANOVA with Tukey's multiple comparison test).

FIGS. 11A-11D depict, in accordance with an embodiment herein, that fungus is associated with the mucosal surface in the mouse cecum. FIG. 11A) Fungi were stained with recombinant soluble Dectin-1 (box) in sections from mouse colon counterstained with DAPI to show nuclear DNA. The expanded inset was further adjusted to show DAPI staining of bacterial cells, demonstrating the colocalization of bacterial and fungal cells in the mucosa. FIG. 11B) Fungi in colonic sections were localized using an anti-yeast antibody (box) that the inventors have confirmed reacts with many species of yeast and counterstained with DAPI. FIG. 11C) Prevalence of fungi in mucosa isolated from ileum, cecum, proximal (prox) and distal (dist) colon of C57BL/6J mice. Fungal rDNA (ITS1-2 region) was analyzed by qPCR and normalized to β-actin DNA. FIG. 11D) The presence of fungi was measured (as in C) in stool samples from the indicated species.

FIGS. 12A-12E depict, in accordance with an embodiment herein, that Dectin-1 regulates severity of colitis. FIG. 12A) Body weight in wild type (WT) and Clec7a−/− mice treated with 2.5% DSS for 7 days and kept on water for 4 additional days. FIG. 12B) Histology score and FIG. 12C) haematoxylin and eosin stained colon sections was determined 4 days after the 7 days of DSS treatment. FIG. 12D) Dot plots show the percentage of IL-17 and IFN-γ producing CD4+ T cells isolated from large intestine Lamina propria (LI-LP) and mesenteric lymph nodes (MLN) 4 days after the 7 days of DSS treatment. FIG. 12E) LI-LP and MLN cells were cultured with antibodies against CD3 and CD28. The production of IL-17 and IFN-γ was measured by ELISA.

FIG. 13 depicts, in accordance with an embodiment herein, the lack of ASCA response in Dectin-1 knockout mice. ASCA titers in serum of DSS-treated mice were measured.

FIG. 14 depicts, in accordance with an embodiment herein, the CLEC7A “AG/AG” haplotype associates with severity of disease as indicated by earlier progression to colectomy. Haplotypes were tested for association with time to surgery by fitting the MRUC/non-MRUC and time-to surgery with a Cox proportional hazards model.

FIGS. 15A-15B depict, in accordance with an embodiment herein, murine commensal fungi. DNA was isolated from murine fecal specimens (FIG. 15A) and food (FIG. 15B) and fungal microbiome analysis was performed using multitag 454 pyrosequencing of ITS1-2 rDNA. The taxonomic distribution of the most abundant fungal species is shown. There was very little overlap (red arrows) suggesting that the majority of the fungi detected in murine fecal specimens derived from commensal populations. Nearly identical data were obtained when samples were sequenced on the Illumina Next-gen platform indicating that the shorter read length (but greater throughput) does not compromise species identifications.

FIG. 16 depicts, in accordance with an embodiment herein, human fungal mycobiome. Fungal species identified in samples from a pilot test of two non-diseased volunteers. The most prevalent Saccharomyces cerevisiae (Baker's yeast) was removed to highlight other diversity.

FIGS. 17A-17C depict, in accordance with an embodiment herein, analysis of CLEC7A SNPs and haplotypes. FIG. 17A) Location of CLEC7A SNPs. Cartoon of CLEC7A gene with track showing position on Human Genome Build 37, track showing the location of the 5 SNPs examined in this study, and track showing exon/intron structure of transcript variants. Arrows indicate locations for SNPS in our evaluation FIG. 17B) Linkage disequilibrium between CLEC7A SNPs. Using Haploview v4, linkage disequilibrium between CLEC7A SNPs was plotted; red diamonds without numbers indicate r2=1. The rs2078178-rs16910631 haplotype is marked as “Block 1”. (C) CLEC7A haplotypes and MRUC. Haplotypes were assigned using the expectation-maximization algorithm in Haploview and tested for association with MRUC with results as shown. Permutation test for the CLEC7A haplotype AG gave p value=0.0005.

FIGS. 18A-18B depict, in accordance with an embodiment herein, CARD9 overexpression & risk genotype. Human macrophages from donors homozygous for the protective CARD9 allele (controls) or the risk allele (that causes a S12N coding region change) were analyzed for CARD9 protein expression. FIG. 18A) Representative immunoblot. FIG. 18B) Quantitative compilation of the data.

FIG. 19 depicts innate antifungal receptors and signaling through CARD9. Proteins/processes linked to IBD through genetic studies are indicated with asterisks.

FIG. 20A-20D depict Malassezia spp. are enriched in the colonic mucosa of Crohn's Disease patients carrying the CARD9S12N allele. A) Mucosa-associated fungal microbiome sequencing was performed in healthy controls (HC) and Crohn's Disease patients (CD). MaAsLin analysis revealed a strong association of Malassezia with disease, particularly in patients with ileocolonic CD. Notched box plots show individual arcsine square-root transformed relative abundance's median and confidence intervals. B) Presence or absence of the CD-associated variant of CARD9 was assessed for association with fungal genera. Fungal genera approaching or exceeding statistical significance (*=Bonferroni-corrected 6.7×10⁻⁴) are listed. Malassezia was associated with the presence of the CARD9 IBD risk allele. C) Box-and-whisker plot showing relative abundance of Malassezia in sigmoid colon CD samples according to the CARD9 S12N SNP genotype. Malassezia was markedly enriched in patients with the CARD9 risk allele (CARD9 AA). (*, p<0.05; one-way ANOVA with Tukey's multiple comparison test). D) Human peripheral blood-derived dendritic cells were prepared from 8-15 pairs of healthy donors determined to be homozygous for the S12N CARD9 A (risk) or G (protective) alleles. Cells were stimulated (MOI 10) with the indicated yeasts for 24 hours and production of TNF-α and IL-8 were measured by ELISA. Each dot is an individual patient (measured in duplicate). (*, p<0.05; **, p<0.01; ***, p<0.001; one-way ANOVA with Tukey's multiple comparison test.

FIG. 21 depicts CARD9 polymorphisms and their effects. The CARD9 gene comprises 13 exons. A common single nucleotide polymorphism in exon 2 causes one allele to code for a serine in position 12 and the others to code for an arginine. A second polymorphism at the end of exon 11 linked to the CARD9^{N12} variant results in production of a splice variant lacking exon 11 and production of a protein lacking function. While the N12 variant confers increased risk of developing Crohn's Disease, the truncation variant is protective. (Population distribution from ensembl.org)

FIG. 22 depicts Efficacy of azole drug on colonic Malassezia in mice. Mice were treated with oral fluconazole for 1 week, and Malassezia levels were measured in the stool by rDNA sequencing.

FIG. 23 depicts a clinical trial and project overview.

FIG. 24A-24D Antifungal treatment affects bacterial and fungal microbiota in mice. Mice were treated or not for 3 weeks with fluconazole or amphotericin B in their drinking water, DNA was isolated from stool, and fungal and bacterial communities were assessed by sequencing ITS1 and 16S rDNA regions respectively. A) Principal Coordinates Analysis of fungal microbiota confirms that antifungal drugs alter the fungal community. B) Cladogram showing LEfSe analysis of 16S sequences reveals changes in bacterial microbiota after antifungal treatment. C) Examples of fungi (genera) up- and down-regulated upon antifungal treatment. D) Examples of bacteria (families) regulated upon antifungal treatment. * p<0.05; ** p<0.01; *** p<0.001; LDA scores from LEfSe analysis.

FIG. 25A-25D depicts serological data. A) M. restricta was stained with human sera from CD patients previously characterized as “low” or “high” for ASCA, and levels of anti-Malassezia IgA (left) and IgG (right) were evaluated by flow cytometry. ASCA “high” sera positively stain M. restricta. B)M. restricta and M. globosa secrete lipases that are essential for acquiring long chain fatty acids that the yeast need to grow. C) M. restricta and M. globosa Lip1 were recombinantly produced in E. coli. and protein purity was assessed by SDS-PAGE and Coomassie staining. D) M. restricta Lip1-reactive IgA was measured by ELISA in sera from CARD9^{S12N} CD patients. A higher proportion of ASCA+ patients have IgA against Malassezia Lip1. P values by Mann-Whitney U Test. Notches indicate 95% confidence interval and whiskers extend no further than 1.5 times IQR from the hinge.

### DETAILED DESCRIPTION

Unless defined otherwise, technical and scientific terms used herein have the same meaning as commonly understood by one of ordinary skill in the art to which this inventive subject matter belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed, Cold Spring Harbor Laboratory Press (Cold Spring Harbor, N.Y. 2012), provide one skilled in the art with a general guide to many of the terms used in the present application.

One skilled in the art will recognize many methods and materials similar or equivalent to those described herein, which could be used in the practice of the present embodiments. Indeed, the present embodiments are in no way limited to the methods and materials described. For purposes of the present embodiments, the following terms are defined below.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 5%. If specifically indicated in a claim, the term “about” can refer to an amount that is near the stated amount by 10%, 9%, 8%, 7%, 6%, 5%, 4%, 3%, 2% or 1%.

The terms “increased,” or “increase” are used herein to generally mean an increase by a statically significant amount; in some embodiments, the terms “increased,” or “increase,” mean an increase of at least 10% as compared to a reference level, for example an increase of at least 10%, at least 20%, or at least 30%, or at least 40%, or at least 50%, or at least 60%, or at least 70%, or at least 80%, or at least 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, 60-fold, 70-fold, 80-fold, 90-fold, 100-fold, at least 100-fold, at least 1000-fold or more as compared to a reference level.

The terms, “decreased” or “decrease” are used herein generally to mean a decrease by a statistically significant amount. In some embodiments, “decreased” or “decrease” means a reduction by at least 10% as compared to a reference level, for example a decrease by at least 20%, or at least 30%, or at least 40%, or at least 50%, or at least 60%, or at least 70%, or at least 80%, or at least 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The term, “genetic risk variant” as disclosed herein, refers to a variation in a polynucleotide sequence of a gene. In some instances, the genetic risk variant comprises a single nucleotide variant (SNV) at an allele. In some instances, the single nucleotide variant comprises a single nucleotide polymorphism (SNP). In some instances, the genetic risk variant comprises a substitution, insertion, or deletion, of a nucleobase (indel).

“Linkage disequilibrium,” or “LD,” as used herein refers to the non-random association of alleles at different loci in a population. LD may be defined by a D′ value corresponding to the difference between an observed and expected allele frequencies in the population (D=Pab−PaPb), which is scaled by the theoretical maximum value of D. LD may be defined by an r² value corresponding to the difference between an observed and expected allele frequencies in the population (D=Pab−PaPb), which is scaled by the individual frequencies of the different loci.

“Treatment” and “treating” as used herein refer to both therapeutic treatment and prophylactic or preventative measures, wherein the object is to prevent or slow down (lessen) the targeted pathologic condition, prevent the pathologic condition, pursue or obtain good overall survival, or lower the chances of the individual developing the condition even if the treatment is ultimately unsuccessful. In some aspects provided herein, subjects in need of treatment include those already with a disease or condition, as well as those susceptible to develop the disease or condition or those in whom the disease or condition is to be prevented. The disease or condition may comprise an inflammatory disease or condition, thiopurine toxicity or disease related to thiopurine toxicity, non-response to anti-TNF therapy, steroids or immunomodulators.

Non-limiting examples of “biological sample” include any material from which nucleic acids and/or proteins can be obtained. As non-limiting examples, this includes whole blood, peripheral blood, plasma, serum, saliva, mucus, urine, semen, lymph, fecal extract, stool, cheek swab, cells, intestinal aspirate or other bodily fluid or tissue, including but not limited to tissue obtained through surgical biopsy or surgical resection. In various embodiments, the sample comprises tissue from the large and/or small intestine. In various embodiments, the large intestine sample comprises the tissue or cells from the cecum, colon (the ascending colon, the transverse colon, the descending colon, and the sigmoid colon), rectum and/or the anal canal. In some embodiments, the small intestine sample comprises tissue or cells from the duodenum, jejunum, and/or the ileum. Alternatively, a sample can be obtained through primary patient derived cell lines, or archived patient samples in the form of preserved samples, or fresh frozen samples.

Provided throughout this application are kits, compositions and methods for the characterization and treatment of a disease or condition. In some embodiments, the disease or condition comprises inflammatory bowel disease (IBD). Also provided are methods of detection of genetic risk variants, biomarkers, and/or serological markers. It should be understood that kits and compositions disclosed herein may be used according to, or for, methods described herein. Conversely, methods disclosed herein may appropriately employ compositions disclosed herein.

### Genetic Risk Variants

Aspects disclosed herein provide genetic risk variants that are associated with a particular disease or condition, or a subclinical phenotype of the disease or condition. In some embodiments, one or more genetic risk variants is associated with Crohn's disease (CD). In some embodiments, the subclinical phenotype comprises intestinal inflammation, or colitis. In some embodiments, the disease or condition comprises medically refractor CD. In some embodiments, the disease or condition comprises medically refractory UC. In some embodiments, the genetic risk variants are also associated with a microbiome comprising fungi within an intestine of a subject.

A non-limiting example of fungal genera include, Malassezia, Cladosporium, Aureobasidium, Fusarium, Candida, Pichia, and Saccharomyces. Non-limiting examples of species of fungi include S. cerevisiae, M. restricta, M. pachydermatis, M. furfur, and M. globosa, C. albicans. In some embodiments, the presence of the fungi disclosed herein is indicative of a severe form of the disease or condition.

In some embodiments, the characteristic of a subject having the particular disease or condition that is related to the presence of a genotype provided herein is increased or decreased level of CARD9 or Dectin-1A expression or activity, as compared to a subject who does not express the one or more genetic risk variants. In some embodiments, the characteristic of a subject having the particular disease or condition that is related to the presence of a one or more genetic risk variants provided herein is suitability for treatment with a modulator of CARD9 and/or Dectin-1A, or an antimycotic agent, such as the active agents disclosed herein.

The genotypes presented herein may be determined by analyzing a biological sample obtained from a subject. The subject may be human. In some embodiments, the biological sample is obtained from a subject having an inflammatory disease, a fibrostenotic disease, a fibrotic disease, or a combination thereof. In some cases, the biological sample is obtained from tissue biopsy, fecal specimen, blood, serum, plasma, sweat, hair, tears, urine, and other similar techniques. In some cases, the fecal specimen is obtained by way of a stool sample. In some cases, the fecal specimen is obtained from the mucosal surface of the intestine. In some instances, the fecal specimen is obtained from the mucosal surface of the intestine by way of a water-lavage. In some cases, the genetic specimen is obtained for a biopsy, e.g., from the intestinal track of the subject.

In some embodiments, the one or more genetic risk variants comprises a single nucleotide polymorphism (SNP) at a gene locus. In some embodiments, the one or more genetic risk variants comprise an insertion or deletion (indel) of a nucleobase at a gene locus. In some embodiments, the one or more genetic risk variants is located at a gene locus comprising CARD9 and/or CLEC7A. In some embodiments, the SNP and/or indel is represented by an “rs” number, which refers to the accession of reference cluster of one more submitted SNPs or indels in the FASTA bioinformatics database, and which is characterized by a FASTA sequence that comprises the total number of nucleobases from 5′ to 3′, including the variation, that was submitted. In some embodiments, the SNP and/or indel is further defined by the position of the risk allele and/or insertion of deletion, respectively, within this sequence, which is always the 5′ length of the sequence plus 1. In some embodiments, the SNP comprises a risk allele at rs4077515, a risk allele at rs10870077, a risk allele at rs2078178, a risk allele at rs795945, a risk allele at rs11053603, a risk allele at rs11053624, and/or a risk allele at rs16910631. In some embodiments, the SNP comprises a SNP in linkage disequilibrium with the SNPs disclosed herein. In some embodiments, risk allele at rs4077515 comprises an “A”. In some embodiments, the risk allele at rs10870077 comprises a “C”. In some embodiments, the risk allele at rs2078178 comprises a “G” or an “A”. In some embodiments, the risk allele at rs16910631 comprises a “G”. Further provided is a haplotype at the CLEC7A gene locus comprises rs2078178 and rs16910631. Also provided is a haplotype at the CARD9 gene locus comprising rs4077515 and rs10870077. A “haplotype,” as used herein refers, in some instances, to a set of SNPs that tend to be inherited together. Exemplary SNP sequences are provided within SEQ ID NOS: 1-7. The “Y” indicates the position of the risk allele in the context of each polynucleotide sequence of SEQ ID NOS: 1-7.

### Biomarkers

Aspects disclosed herein provide methods of diagnosing, characterizing, and treating a disease or condition in a subject, provided one or more biomarkers is detected in a biological sample obtained from the subject. A biomarker is a biological substance in an organism, the presence and/or quantity of which, is indicative of a particular condition of the organism. In some instances, the genes CLEC7 (Dectin-1A) and/or CARD9, or gene expression products thereof, are biomarkers. In some instances, an increased level or activity of CARD9 is associated with a disease or condition. In some instances, an increased level or activity of CLEC7A (Dectine-1A) is associated with a disease or condition. In some instances, the disease or condition comprises inflammatory bowel disease (IBD), Crohn's disease (CD), or ulcerative colitis (UC). In some instances, the ulcerative colitis is medically refractory UC (mrUC). In some instances, the CD is medically refractory. In some instances, the disease or condition is characterized by an increase in colitis or gut inflammation. In some instances, an increase or a decrease in a level of one or more biomarkers is associated with a presence of one or more genetic risk variants, such as those disclosed herein. In some instances, the increase or decrease in the level or activity of a biomarker is caused by expression of one or more genetic risk variants disclosed herein. In some instances, a presence of a risk allele A at rs4077515 causes a serine to asparagine mutation at codon 12 in the CARD9 protein, thereby activating CARD9 protein. In some instances, the presence of a risk allele A at rs4077515 (heterozygous) or two risk alleles AA at rs4077515 (homozygous) may be detected in a biological sample obtained from a subject in order to determine whether there is an increase in expression of activity of CARD9.

### Serological Markers

A serological marker is a type of biomarker, such as an autoantigen, that represent a serological response to microbial antigens in the body. In some instances, the serological marker comprises an antibody against a species of fungi belonging to a fungal genus comprising Malassezia, Cladosporium, Aureobasidium, Fusarium, Candida, Pichia, and/or Saccharomyces. Non-limiting examples of species of the above fungal genera, include S. cerevisiae, M. restricta, M. pachydermatis, M. furfur, and M. globosa, C. albicans. In some embodiments, the serological marker is an enzyme of a fungus; for example, a lipase. In particular embodiments, the serological marker is Malassezia lipase 1, or Cladosporium lipase 1.

Aspects disclosed herein provide serological markers that are associated with a particular disease or condition, or a subclinical phenotype of the disease or condition. In some embodiments, one or more serological markers is associated with Crohn's disease (CD). In some embodiments, the one or more serological markers is associated with a subclinical phenotype comprising intestinal inflammation, or colitis. In some embodiments, the one or more serological markers is also associated with a presence of one or more genetic risk variants. A non-limiting example of genetic risk variants includes, a genetic risk variant, or haplotype, at the CARD9 gene locus and/or the CLEC7A gene locus. In some embodiments, the genetic risk variant, comprises one or more single nucleotide polymorphisms (SNPs) disclosed herein. In some embodiments, the SNP comprises a risk allele at rs4077515, a risk allele at rs10870077, a risk allele at rs2078178, a risk allele at rs795945, a risk allele at rs11053603, a risk allele at rs11053624, and/or a risk allele at rs16910631. In some embodiments, risk allele at rs4077515 comprises an “A”. In some embodiments, the risk allele at rs10870077 comprises a “C”. In some embodiments, the risk allele at rs2078178 comprises a “G” or an “A”. In some embodiments, the risk allele at rs16910631 comprises a “G”. In some instances, the haplotype comprising rs2078178 and rs16910631, or rs4077515 and rs10870077.

### Methods of Treatment

Aspects disclosed herein provide methods of treating an individual having a disease or condition. In some cases, the disease or condition is an inflammatory disease, fibrostenotic disease, and/or fibrotic disease. Non-limiting examples of inflammatory diseases include diseases of the gastrointestinal tract, liver, and/or gallbladder; including Crohn's disease (CD) and ulcerative colitis, systemic lupus erythematosus (SLE), and rheumatoid arthritis. An exemplary fibrotic disease is primary sclerosing cholangitis (PSC). In some instances, the disease or condition comprises inflammatory bowel disease (IBD), Crohn's disease (CD), or ulcerative colitis (UC). In some instances, the ulcerative colitis is medically refractory UC (mrUC). In some instances, the CD is medically refractory. In some instances, the disease or condition is characterized by an increase in colitis or gut inflammation. In some embodiments, the subject is susceptible to, or is inflicted with, thiopurine toxicity, or a disease caused by thiopurine toxicity (such as pancreatitis or leukopenia). In further embodiments provided, the subject is non-responsive to a therapy comprising anti-TNF alpha therapy, anti-a4-b7 therapy (vedolizumab), anti-IL12p40 therapy (ustekinumab), Thalidomide, or Cytoxin. In some cases, the subject is not responsive to the induction of said therapy. In some cases, the subject loses responsiveness to said therapy after a period of time during treatment.

Various embodiments of the present embodiments provide for a method of treating a disease or condition in a subject comprising administering to the subject a therapeutically effective amount of active agent, provided one or more genetic risk variants, serological markers, and/or biomarkers is detected in a biological sample obtained from the subject. In some embodiments, the active agent comprises an antifungal agent and/or an anti-mycotic treatment. In yet other embodiments, the active agent comprises a targeted therapy, a small molecule, an antibody, gene therapy, an agonist modulator, or an antagonist modulator to Dectin-1 (CLEC7A), or CARD9 or a combination thereof.

Various embodiments of the present invention provide for a method of treating an inflammatory disease or condition in a subject in need thereof, comprising: administering an anti-fungal therapy to the subject in need thereof, wherein the subject in need thereof has a fungus in the gastrointestinal system.

Various embodiments of the present invention provide for a method of treating an inflammatory disease or condition in a subject in need thereof, comprising: requesting the results of a diagnosis regarding the inflammatory disease or condition, wherein the diagnosis is performed by a method of the present invention described herein; and administering an anti-fungal therapy to the subject in need thereof, wherein the subject in need thereof has a fungus in the gastrointestinal system.

Various embodiments of the present invention provide for a method of manipulating the microbiome of a subject having an inflammatory disease or condition, comprising: administering an anti-fungal therapy to the subject, wherein the subject in need thereof has a fungus in the gastrointestinal system.

Various embodiments of the present invention provide for a method of inhibiting or reducing fungal growth in a subject having an inflammatory disease or condition, comprising: administering an anti-fungal therapy to the subject in need thereof, wherein the subject in need thereof has a fungus in the gastrointestinal system.

Various embodiments of the present invention provide for a method of inhibiting or reducing fungal growth in a subject having an inflammatory disease or condition, comprising: requesting the results of a diagnosis regarding the inflammatory disease or condition, wherein the diagnosis is performed by a method of the present invention described herein; and administering an anti-fungal therapy to the subject in need thereof, wherein the subject in need thereof has a fungus in the gastrointestinal system.

In various embodiments, the subject in need thereof has a c-type lectin domain containing 7A (CLEC7A) gene risk variant, a caspase recruitment domain family member 9 (CARD9) gene risk variant or both. In various embodiments, the subject in need thereof has a caspase recruitment domain family member 9 (CARD9) gene risk variant A in rs4077515 resulting in an amino acid substitution S12N. In various embodiments, the subject has an elevated level of a serological marker, as compared to a control level of the serological marker, said serological marker selected from the group consisting of anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globosa antibody, anti-Cladosporium spp. antibody, and a combination thereof.

In various embodiments, the subject has an elevated level of a serological marker, as compared to a control level of the serological marker, said serological marker selected from the group consisting of anti-Malassezia restricta antibody, anti-Malassezia pachydermatis antibody, anti-Malassezia furfur antibody, anti-Malassezia globosa antibody, anti-Cladosporium spp. antibody, and a combination thereof.

In various embodiments, the fungus is Malassezia, Cladosporium or both.

In some embodiments, the subject has an elevated level or a serological marker, as compared to a control level of the serological marker, and said serological marker is an enzyme of a fungus; for example, a lipase. In particular embodiments, the serological marker is Malassezia lipase 1, or Cladosporium lipase 1.

In various embodiments, the inflammatory disease is inflammatory bowel disease. In various embodiments, the inflammatory disease is ulcerative colitis. In various embodiments, the inflammatory disease is Crohn's disease.

In various embodiments, the anti-fungal therapy comprises posaconazole. In various embodiments, the anti-fungal therapy comprises a fungicide agent or a fungistatic agent. In various embodiments, the anti-fungal therapy comprises an antimycotic agent. In various embodiments, the antimycotic agent comprises a polyene, an azole, an echinocandin, an flucytosine, an allylamine, a tolnaftate, or griseofulvin. In various embodiments, the azole comprises triazole, imidazole, clotrimazole, ketoconazole, itraconazole, terconazole, oxiconazole, miconazole, econazole, tioconazole, voriconazole, fluconazole, isavuconazole, itraconazole, pramiconazole, ravuconazole, or posaconazole. In various embodiments, the polyene comprises amphotericin B, nystatin, or natamycin. In various embodiments, the echinocandin comprises caspofungin, anidulafungin, or micafungin. In various embodiments, the allylamine comprises naftifine or terbinafine.

### Modulators of CARD9

In some instances, the treatment comprises administering to the subject an active agent that modulates CARD9 activity or expression. In various embodiments, the inhibitor of CARD9 activity or expression comprises a CARD9 antibody, a small molecule, a direct inhibitor of CARD9, an indirect inhibitor of CARD9, an allosteric modulator of CARD9, an anti-CARD9 antibody or antibody fragment, antibody or antibody fragment that specifically binds to Rubicon, an anti-tripartite Motif Containing 62 (TRIM62) antibody or antibody fragment, an antibody or antibody fragment that specifically binds to B Cell CLL/Lymphoma 10 (BCL10), an inhibitor of CARD9-Rubicon interaction, an inhibitor of CARD9-Tripartite Motif Containing 62 (TRIM62) interaction, an inhibitor of CARD9-B Cell CLL/Lymphoma 10 (BCL10) interaction, a small molecule that specifically binds CARD9 a small molecule that specifically binds to Rubicon, a small molecule that specifically binds to Tripartite Motif Containing 62 (TRIM62), a small molecule that specifically binds to B Cell CLL/Lymphoma 10 (BCL10), an inhibitor of CARD9-Rubicon interaction, an inhibitor of CARD9-Tripartite Motif Containing 62 (TRIM62) interaction, an inhibitor of B Cell CLL/Lymphoma 10 (BCL10)-CARD9 interaction, or a combination thereof. In some other embodiments, the inhibitor of CARD9 activity or expression comprises the small molecule inhibitor BRD5529, BRD4203, BRD8991, BRD4098 or a combination thereof. In some embodiments, the CARD9 antibody recognizes the total CARD9 protein. In other embodiments, the CARD9 antibody recognizes 90%, 80%, 70%, 60%, 50%, 40%, 30%, 20%, or 10% of the total CARD9 protein. In some embodiments, the modulator of CARD9 comprises a stem cell therapy. The stem cell therapy may be embryonic or somatic stem cells. The stem cells may be isolated from a donor (allogeneic) or isolated from the subject (autologous). The stem cells may be expanded adipose-derived stem cells (eASCs), hematopoietic stem cells (HSCs), mesenchymal stem (stromal) cells (MSCs), or induced pluripotent stem cells (iPSCs) derived from the cells of the subject.

### Modulators of Dectin-1A

In some instances, the treatment comprises administering to the subject an antibody or antibody fragment, a small molecule, an allosteric modulator, an agonist, an antagonist, a direct modulator of Dectin-1A, an indirect modulator of Dectin-1A, or a combination thereof. In other embodiments, the treatment is an inhibitor of C-type lectin-like receptors. In various embodiments, the agonist is soluble R-glucan antagonist laminarin. In various other embodiments, the antagonist is soluble R-glucan antagonist laminarin. In some embodiments, the antibody binds to the C-type lectin-like receptors. In some embodiments, the Dectin-1 antibody recognizes the total Dectin-1 protein. In other embodiments, the Dectin-1 antibody recognizes 90%, 80%, 70%, 60%, 50%, 40%, 30%, 20%, or 10% of the total Dectine-1A protein. In some embodiments, the modulator of Dectin-1A comprises a stem cell therapy. The stem cell therapy may be embryonic or somatic stem cells. The stem cells may be isolated from a donor (allogeneic) or isolated from the subject (autologous). The stem cells may be expanded adipose-derived stem cells (eASCs), hematopoietic stem cells (HSCs), mesenchymal stem (stromal) cells (MSCs), or induced pluripotent stem cells (iPSCs) derived from the cells of the subject.

### Antimycotic Agents

In some instances, the treatment comprises administering to the subject an antimycotic agent. In some instances, the antimycotic agent comprises an active agent that inhibits growth of a fungus. In some instances, the antimycotic agent comprises an active agent that kills a fungus. In some embodiments, the antimycotic agent comprises polyene, an azole, an echinocandin, an flucytosine, an allylamine, a tolnaftate, or griseofulvin, or a combination thereof. In other embodiments, the azole comprises triazole, imidazole, clotrimazole, ketoconazole, itraconazole, terconazole, oxiconazole, miconazole, econazole, tioconazole, voriconazole, fluconazole, isavuconazole, itraconazole, pramiconazole, ravuconazole, or posaconazole. In some other embodiments, the polyene comprises amphotericin B, nystatin, or natamycin. In yet other embodiments, the echinocandin comprises caspofungin, anidulafungin, or micafungin. In various other embodiments, the allylamine comprises naftifine or terbinafine.

### Other Active Agents

Treatments useful with the methods described herein include active agents that may be used alone, or in combination with a modulator of CARD9 and/or Dectin-1A, and/or a antimycotic agent. In some embodiments, treatment comprises administering a first active agent and then a modulator of CARD9 and/or Dectin-1A, and/or a antimycotic agent, as described herein. In some embodiments, treatment comprises administering a first active agent and a modulator of CARD9 and/or Dectin-1A, and/or a antimycotic agent, as described herein, together. The combination therapies may be administered within the same day, or may be administered one or more days, weeks, months, or years apart. In some cases, a modulator of CARD9 and/or Dectin-1A, and/or a antimycotic agent, as described herein, is administered if the subject is determined to be non-responsive to a first line of therapy, e.g., such as TNF inhibitor and/or steroid. Such determination may be made by treatment with the first line therapy and monitoring of disease state and/or diagnostic determination that the subject would be non-responsive to the first line therapy.

In some embodiments, the other active agent comprises an anti-TNF therapy, e.g., an anti-TNFα therapy. In some embodiments, the other active agent comprises a second-line treatment to an anti-TNF therapy. In some embodiments, the other active agent comprises an immunosuppressant, or a class of drugs that suppress, or reduce, the strength of the immune system. In some embodiments, the immunosuppressant is an antibody. Non-limiting examples of immunosuppressant active agents include STELARA® (ustekinumab) azathioprine (AZA), 6-mercaptopurine (6-MP), methotrexate, cyclosporin A. (CsA).

In some embodiments, the other active agent comprises a selective anti-inflammatory drug, or a class of drugs that specifically target pro-inflammatory molecules in the body. In some embodiments, the anti-inflammatory drug comprises an antibody. In some embodiments, the anti-inflammatory drug comprises a small molecule. Non-limiting examples of anti-inflammatory drugs include ENTYVIO (vedolizumab), corticosteroids, aminosalicylates, mesalamine, balsalazide (Colazal) and olsalazine (Dipentum).

In some embodiments, the other active agent comprises a small molecule. The small molecule may be used to treat the inflammatory diseases or conditions, or fibrostenonic or fibrotic disease. Non-limiting examples of small molecules include Otezla® (apremilast), alicaforsen, or ozanimod (RPC-1063).

In some embodiments the other active agent comprises an inhibitor of TL1A expression or activity. In some cases, the inhibitor of TL1A expression or activity is effective to inhibit TL1A-DR3 binding. In some embodiments, the inhibitor of TL1A expression or activity comprises an allosteric modulator of TL1A. An allosteric modulator of TL1A may indirectly influence the effects TL1A on DR3, or TR6/DcR3 on TL1A or DR3. The inhibitor of TL1A expression or activity may be a direct inhibitor or indirect inhibitor. Non-limiting examples of an inhibitor of TL1A expression include RNA to protein TL1A translation inhibitors, antisense oligonucleotides targeting the TNFSF15 mRNA (such as miRNAs, or siRNA), epigenetic editing (such as targeting the DNA-binding domain of TNFSF15, or post-translational modifications of histone tails and/or DNA molecules). Non-limiting examples of an inhibitor of TL1A activity include antagonists to the TL1A receptors, (DR3 and TR6/DcR3), antagonists to TL1A antigen, and antagonists to gene expression products involved in TL1A mediated disease. Antagonists as disclosed herein, may include, but are not limited to, an anti-TL1A antibody, an anti-TL1A-binding antibody fragment, or a small molecule. The small molecule may be a small molecule that binds to TL1A or DR3. The anti-TL1A antibody may be monoclonal or polyclonal. The anti-TL1A antibody may be humanized or chimeric. The anti-TL1A antibody may be a fusion protein. The anti-TL1A antibody may be a blocking anti-TL1A antibody. A blocking antibody blocks binding between two proteins, e.g., a ligand and its receptor. Therefore, a TL1A blocking antibody includes an antibody that prevents binding of TL1A to DR3 or TR6/DcR3 receptors. In a non-limiting example, the TL1A blocking antibody binds to DR3. In another example, the TL1A blocking antibody binds to DcR3. In some cases, the TL1A antibody is an anti-TL1A antibody that specifically binds to TL1A.

Non-limiting methods for determining whether an anti-TL1A antibody binds to the same region of a reference antibody are known in the art. An exemplary method comprises a competition assay. For instance, the method comprises determining whether a reference antibody can compete with binding between the reference antibody and the TL1A protein or portion thereof, or determining whether the reference antibody can compete with binding between the reference antibody and the TL1A protein or portion thereof. Exemplary methods include use of surface plasmon resonance to evaluate whether an anti-TL1A antibody can compete with the binding between TL1A and another anti-TL1A antibody. In some cases, surface plasmon resonance is utilized in the competition assay.

### Methods of Determining Analyte Levels in Particular Subjects and Method of Diagnosis

Various embodiments of the present invention provides for a method of determining a level of a fungus, a fungal antigen, an anti-fungal antibody, or an enzyme of a fungus, in a subject in need thereof, comprising obtaining a biological sample from the subject; subjecting the biological sample to an assay suitable to detect the fungus, the fungal antigen the anti-fungal antibody, or the enzyme of a fungus, in the biological sample; and measuring the level of the fungus, the fungal antigen, the anti-fungal antibody, or the enzyme of the fungus, to determine the level of the fungus, the fungal antigen, the anti-fungal antibody, or the enzyme of the fungus.

Various embodiments of the present invention provide for a method of diagnosing an inflammatory disease or condition in a subject, comprising: obtaining a biological sample from the subject; subjecting the biological sample to an assay suitable to detect the fungus, the fungal antigen the anti-fungal antibody, or the enzyme of a fungus, in the biological sample; measuring the level of the fungus, the fungal antigen, the anti-fungal antibody, or the enzyme of the fungus; and diagnosing the subject with the inflammatory disease or condition when an elevated level of the fungus, the fungal antigen, the anti-fungal antibody, or the enzyme of a fungus as compared to each respective control level, is measured in the biological sample.

In various embodiments, detecting the presence of the fungal antigen comprises contacting an antibody capable specifically binding to the fungal antigen to the biological sample to form a binding complex, and detecting the presence of the binding complex.

In various embodiments, detecting the presence of anti-fungal antibody comprises contacting an antibody capable specifically binding to the anti-fungal antibody to form a binding complex, and detecting the presence of the binding complex.

In various embodiments, detecting the presence of the enzyme of a fungus comprise contacting an antibody capable specifically binding to the enzyme to form a binding complex, and detecting the presence of the binding complex.

In various embodiments, the biological sample is selected from the group consisting of blood plasma, blood serum, stool, intestinal aspirate, intestinal tissue sample, intestinal mucosa, intestinal mucus and combinations thereof.

In various embodiments, the fungus is Malassezia, Cladosporium, or both. In various embodiments, the fungal antigen is from Malassezia, Cladosporium, or both. In various embodiments, the anti-fungal antibody is an antibody capable of specifically binding to Malassezia, Cladosporium, or both, or specifically binding to an antigen of Malassezia, Cladosporium, or both. In various embodiments, the enzyme is a lipase.

In various embodiments, the subject in need thereof has a c-type lectin domain containing 7A (CLEC7A) gene risk variant, a caspase recruitment domain family member 9 (CARD9) gene risk variant or both.

In various embodiments, the subject in need thereof has a caspase recruitment domain family member 9 (CARD9) gene risk variant A in rs4077515 resulting in an amino acid substitution S12N. In various embodiments, the subject has an elevated level of a serological marker, as compared to a control level of the serological marker, said serological marker selected from the group consisting of anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globosa antibody, anti-Cladosporium spp. antibody, and a combination thereof. In some embodiments, the subject has an elevated level of a serological marker, as compared to a control level of the serological marker, said serological marker is an enzyme of a fungus; for example, a lipase. In particular embodiments, the serological marker is Malassezia lipase 1, or Cladosporium lipase 1.

### Methods of Monitoring Treatment

In certain embodiments, described herein are methods for evaluating an effect of a treatment described herein. In some instances, the treatment comprises administration with a modulator of CARD9 and/or modulator of Dectin-1A, and/or an antimycotic agent, and optionally, one or more additional active agents. In some instances, the treatment is monitored by evaluating the quantity of a CARD9 and/or Dectin-1A, and/or fungal organism, in the subject prior to and/or after administration of an active agent, such as a modulator of CARD9 and/or Dectin-1A, and/or a antimycotic agent. In some embodiments, the fungal organism comprises Malassezia spp. (e.g. Malassezia globosa) or Cladosporium spp. In some embodiments, the CARD9 protein comprises a serine to asparagine substitution at amino acid number 12 (S12N mutation). In some embodiments, the treatment is monitored by evaluating the quantity or presence of the S12N mutation. In some embodiments, the modulator of CARD9 comprises an inhibitor. In some embodiments, the inhibitor of CARD9 comprises BRD5529, BRD4203, BRD8991, BRD4098 or a combination thereof. In various embodiments, the quantity of the CARD9 S12N mutation in the subject prior to and/or after administration of an active agent is used to direct continued treatment of the subject. In instances wherein the active agent is not therapeutically effective or is not providing a sufficient alleviation of the disease or condition, then method further comprises ceasing the therapeutic treatment. In other embodiments, the method further comprises administering an additional active agent along with the initial active agent.

### Pharmaceutical Compositions, Formulations, and Methods of Administration

In one aspect, a method for treating any of the diseases or conditions described herein in a subject in need of such treatment, involves administration of pharmaceutical compositions that include an active agent described herein, in therapeutically effective amounts to said subject. In some embodiments, an active agent described herein is used in the preparation of medicaments for treating a disease or condition disclosed herein. In some embodiments, the disease or condition comprises an inflammatory disease, a fibrostenotic disease, and/or a fibrotic disease. Pharmaceutical compositions as used herein include compositions comprising a modulator of CARD9 and/or Dectin-1A and optionally an additional active agent, such as the other active agents disclosed herein. In various embodiments, the modulator of CARD9 is an inhibitor of CARD9. In various embodiments, the modulator of Dectin-1A is an inhibitor of Dectin-1A. In other embodiments, the inhibitor of CARD9 comprises, but is not limited to, BRD5529, BRD4203, BRD8991, BRD4098 or a combination thereof.

In certain embodiments, the compositions containing the active agent described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial. In some cases, a modulator of CARD9 and/or Dectin-1A, and/or a antimycotic agent is administered to a patient suffering from an inflammatory disease, fibrostenotic disease, and/or fibrotic disease.

In prophylactic applications, compositions containing an active agent described herein are administered to a patient susceptible to or otherwise at risk of a particular disease, disorder or condition, e.g., an inflammatory disease, fibrostenotic disease, and/or fibrotic disease. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a modulator of CARD9 and/or Dectin-1A, as described herein, in order to prevent a return of the symptoms of the disease or condition.

In certain embodiments wherein the patient's condition does not improve, upon the doctor's discretion the administration of active agent is administered chronically, that is, for an extended period of time, including throughout the duration of the patient's life in order to ameliorate or otherwise control or limit the symptoms of the patient's disease or condition.

In certain embodiments wherein a patient's status does improve, the dose of active agent being administered may be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

In certain embodiments, the dose of drug being administered may be temporarily reduced or temporarily suspended for a certain length of time (i.e., a “drug diversion”). In specific embodiments, the length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given active agent that corresponds to such an amount varies depending upon factors such as the particular active agent, disease condition and its severity, the identity (e.g., weight, sex) of the subject in need of treatment, but can nevertheless be determined according to the particular circumstances surrounding the case, including, e.g., the specific agent being administered, the route of administration, the condition being treated, and the subject or host being treated. In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously (or over a short period of time) or at appropriate intervals, for example as two, three, four or more sub-doses per day.

In some embodiments, as a patient is started on a regimen of an active agent, the patient is also weaned off (e.g., step-wise decrease in dose) a second treatment regimen.

In one embodiment, the daily dosages appropriate for a modulator of CARD9 is determined by one of skill in the art. In some embodiments, the CARD9 modulator, as described herein, can range from about 0.01 to about 10 mg/kg per body weight. In specific embodiments, an indicated daily dosage in a large mammal, including, but not limited to, humans, is in the range from about 0.5 mg to about 1000 mg, conveniently administered in divided doses, including, but not limited to, up to four times a day. In some embodiments, the daily dosage is administered in extended release form. In certain embodiments, suitable unit dosage forms for oral administration comprise from about 1 to 500 mg active ingredient. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the active agent used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the active agent described herein lies within a range of circulating concentrations that include the ED₅₀ with minimal toxicity. In certain embodiments, the daily dosage range and/or the unit dosage amount varies within this range depending upon the dosage form employed and the route of administration utilized.

Disclosed herein are active agents formulated into pharmaceutical compositions. The pharmaceutical composition may comprise a modulator of CARD9 and/or Dectin-1A, and/or an antimycotic agent. The pharmaceutical composition may comprise an antibody. The pharmaceutical composition may comprise an anti-CARD9 antibody.

Pharmaceutical compositions are formulated in a conventional manner using one or more pharmaceutically acceptable inactive ingredients that facilitate processing of the active agent into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), herein incorporated by reference for such disclosure.

Provided are pharmaceutical compositions that include a CARD9 modulators, Dectin-1a modulators, and/or antimycotic agents, as described herein, and at least one pharmaceutically acceptable inactive ingredient. Optionally, the compositions include other active agent as discussed herein. In some embodiments, the active agents described herein are administered as pharmaceutical compositions in which the active agents are mixed with other active ingredients, as in combination therapy. In some embodiments, the pharmaceutical compositions include other medicinal or pharmaceutical agents, carriers, adjuvants, preserving, stabilizing, wetting or emulsifying agents, solution promoters, salts for regulating the osmotic pressure, and/or buffers. In some embodiments, the pharmaceutical compositions include other therapeutically valuable substances.

A pharmaceutical composition, as used herein, refers to a mixture of an active agent, e.g., a modulator of CARD9 and/or Dectin-1A, and/or a antimycotic agent, with other chemical components (i.e., pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. Optionally, the compositions include two or more active agents as discussed herein. In practicing the methods of treatment or use provided herein, therapeutically effective amounts of active agents described herein are administered in a pharmaceutical composition to a mammal having a disease, disorder, or condition to be treated, e.g., an inflammatory disease, fibrostenotic disease, and/or fibrotic disease. In some embodiments, the mammal is a human. A therapeutically effective amount can vary widely depending on the severity of the disease, the age and relative health of the subject, the potency of the active agent used and other factors. The active agents can be used singly or in combination with one or more active agents as components of mixtures.

The pharmaceutical formulations described herein are administered to a subject by appropriate administration routes, including but not limited to, oral, parenteral (e.g., intravenous, subcutaneous, intramuscular), intranasal, buccal, topical, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including an active agent, e.g., modulator of CARD9 and/or Dectin-1A, are manufactured in a conventional manner, such as, by way of example only, by means of conventional mixing, dissolving, granulating, dragee-making, levigating, emulsifying, encapsulating, entrapping or compression processes. Optionally, the compositions include another active agent, e.g., one as discussed herein.

The pharmaceutical compositions may include at least an active agent, e.g., modulator of CARD9 and/or Dectin-1A, and/or a antimycotic agent as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, active agents exist in unsolvated form or in solvated forms with pharmaceutically acceptable solvents such as water, ethanol, and the like. The solvated forms of the active agents are also considered to be disclosed herein.

In some embodiments, an active agent exists as a tautomer. All tautomers are included within the scope of the agents presented herein. As such, it is to be understood that an active agent or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound.

In some embodiments, an active agent exists as an enantiomer, diastereomer, or other stereoisomeric form. The agents disclosed herein include all enantiomeric, diastereomeric, and epimeric forms as well as mixtures thereof.

In some embodiments, active agents described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be an active agent described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the active agent. In certain embodiments, a prodrug is enzymatically metabolized by one or more steps or processes to the biologically, pharmaceutically or therapeutically active form of the active agent.

Prodrug forms of the active agents, wherein the prodrug is metabolized in vivo to produce an agent as set forth herein are included within the scope of the claims. Prodrug forms of the herein described active agents, wherein the prodrug is metabolized in vivo to produce an agent as set forth herein are included within the scope of the claims. In some cases, some of the active agents described herein may be a prodrug for another derivative or active compound. In some embodiments described herein, hydrazones are metabolized in vivo to produce an active agent.

In certain embodiments, compositions provided herein include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

The pharmaceutical compositions described herein, which include an active agent such a modulator of CARD9 and/or Dectin-1A are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, an active agent as discussed herein, e.g., a modulator of CARD9 and/or Dectin-1A is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. In some cases it is desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections or drips or infusions, an active agent described herein is formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, an active agent is formulated for use as an aerosol, a mist or a powder. Pharmaceutical compositions described herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the active agent described herein and a suitable powder base such as lactose or starch.

Representative intranasal formulations are described in, for example, U.S. Pat. Nos. 4,476,116, 5,116,817 and 6,391,452. Formulations that include an active agent are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the active agents described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active agent doses.

In some embodiments, pharmaceutical formulations of an active agent are in the form of a capsules, including push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active agent is dissolved or suspended in suitable liquids, such as fatty oils, liquid paraffin, or liquid polyethylene glycols. In some embodiments, stabilizers are added. A capsule may be prepared, for example, by placing the bulk blend of the formulation of the active agent inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

All formulations for oral administration are in dosages suitable for such administration. In one aspect, solid oral dosage forms are prepared by mixing an active agent with one or more of the following: antioxidants, flavoring agents, and carrier materials such as binders, suspending agents, disintegration agents, filling agents, surfactants, solubilizers, stabilizers, lubricants, wetting agents, and diluents. In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the pharmaceutical formulation is in the form of a powder. Compressed tablets are solid dosage forms prepared by compacting the bulk blend of the formulations described above. In various embodiments, tablets will include one or more flavoring agents. In other embodiments, the tablets will include a film surrounding the final compressed tablet. In some embodiments, the film coating can provide a delayed release of an active agent from the formulation. In other embodiments, the film coating aids in patient compliance (e.g., Opadry® coatings or sugar coating). Film coatings including Opadry® typically range from about 1% to about 3% of the tablet weight. In some embodiments, solid dosage forms, e.g., tablets, effervescent tablets, and capsules, are prepared by mixing particles of an active agent with one or more pharmaceutical excipients to form a bulk blend composition. The bulk blend is readily subdivided into equally effective unit dosage forms, such as tablets, pills, and capsules. In some embodiments, the individual unit dosages include film coatings. These formulations are manufactured by conventional formulation techniques.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents. Exemplary useful microencapsulation materials include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to active agent the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Buccal formulations that include an active agent are administered using a variety of formulations known in the art. For example, such formulations include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. For buccal or sublingual administration, the compositions may take the form of tablets, lozenges, or gels formulated in a conventional manner.

For intravenous injections, an active agent is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, a pharmaceutical composition described herein is in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethylcellulose (HPMC), hydroxypropylmethylcellulose phthalate, hydroxypropylmethylcellulose acetate stearate (HPMCAS), sucrose, xylitol, lactitol, mannitol, sorbitol, sodium chloride, polyethylene glycol, and the like.

Suitable disintegrants for use in the solid dosage forms described herein include, but are not limited to, natural starch such as corn starch or potato starch, a pregelatinized starch, or sodium starch glycolate, a cellulose such as methylcrystalline cellulose, methylcellulose, microcrystalline cellulose, croscarmellose, or a cross-linked cellulose, such as cross-linked sodium carboxymethylcellulose, cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a gum such as agar, guar, locust bean, Karaya, pectin, or tragacanth, sodium starch glycolate, bentonite, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose, hydroxypropylmethylcellulose, hydroxypropylmethylcellulose acetate stearate, hydroxyethylcellulose, hydroxypropylcellulose, ethylcellulose, and microcrystalline cellulose, microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose, glucose, dextrose, molasses, mannitol, sorbitol, xylitol, lactose, a natural or synthetic gum such as acacia, tragacanth, ghatti gum, mucilage of isapol husks, starch, polyvinylpyrrolidone, larch arabogalactan, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Binder levels of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxypropylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms of the pharmaceutical compositions described herein. The amounts of such additives can be readily determined by one skilled in the art, according to the particular properties desired.

In various embodiments, the particles of an active agents and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In other embodiments, a powder including an active agent is formulated to include one or more pharmaceutical excipients and flavors. Such a powder is prepared, for example, by mixing the active agent and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared. Effervescent salts have been used to disperse medicines in water for oral administration.

In some embodiments, the pharmaceutical dosage forms are formulated to provide a controlled release of an active agent. Controlled release refers to the release of the active agent from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein are formulated as enteric coated delayed release oral dosage forms, i.e., as an oral dosage form of a pharmaceutical composition as described herein which utilizes an enteric coating to affect release in the small intestine or large intestine. In one aspect, the enteric coated dosage form is a compressed or molded or extruded tablet/mold (coated or uncoated) containing granules, powder, pellets, beads or particles of the active ingredient and/or other composition components, which are themselves coated or uncoated. In one aspect, the enteric coated oral dosage form is in the form of a capsule containing pellets, beads or granules, which include an active agent that are coated or uncoated.

Any coatings should be applied to a sufficient thickness such that the entire coating does not dissolve in the gastrointestinal fluids at pH below about 5, but does dissolve at pH about 5 and above. Coatings are typically selected from any of the following: Shellac—this coating dissolves in media of pH>7; Acrylic polymers—examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine; Poly Vinyl Acetate Phthalate (PVAP)—PVAP dissolves in pH>5, and it is much less permeable to water vapor and gastric fluids. Conventional coating techniques such as spray or pan coating are employed to apply coatings. The coating thickness must be sufficient to ensure that the oral dosage form remains intact until the desired site of topical delivery in the intestinal tract is reached.

In other embodiments, the formulations described herein are delivered using a pulsatile dosage form. A pulsatile dosage form is capable of providing one or more immediate release pulses at predetermined time points after a controlled lag time or at specific sites. Exemplary pulsatile dosage forms and methods of their manufacture are disclosed in U.S. Pat. Nos. 5,011,692, 5,017,381, 5,229,135, 5,840,329 and 5,837,284. In one embodiment, the pulsatile dosage form includes at least two groups of particles, (i.e., multiparticulate) each containing the formulation described herein. The first group of particles provides a substantially immediate dose of an active agent upon ingestion by a mammal. The first group of particles can be either uncoated or include a coating and/or sealant. In one aspect, the second group of particles comprises coated particles. The coating on the second group of particles provides a delay of from about 2 hours to about 7 hours following ingestion before release of the second dose. Suitable coatings for pharmaceutical compositions are described herein or known in the art.

In some embodiments, pharmaceutical formulations are provided that include particles of an active agent and at least one dispersing agent or suspending agent for oral administration to a subject. The formulations may be a powder and/or granules for suspension, and upon admixture with water, a substantially uniform suspension is obtained.

In some embodiments, particles formulated for controlled release are incorporated in a gel or a patch or a wound dressing.

In one aspect, liquid formulation dosage forms for oral administration and/or for topical administration as a wash are in the form of aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the particles of an active agent, the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

In some embodiments, the liquid formulations also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphatidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state, as defined in The USP Pharmacists' Pharmacopeia (2005 edition, chapter 905), for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch, or sodium starch glycolate; a cellulose such as methylcrystalline cellulose, methylcellulose, croscarmellose, or a cross-linked cellulose, such as cross-linked sodium carboxymethylcellulose, cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein include, for example, hydrophilic polymers, electrolytes, Tween® 60 or 80, PEG, polyvinylpyrrolidone, and the carbohydrate-based dispersing agents such as, for example, hydroxypropylcellulose and hydroxypropyl cellulose ethers, hydroxypropyl methylcellulose and hydroxypropyl methylcellulose ethers, carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer, 4-(1,1,3,3-tetramethylbutyl)-phenol polymer with ethylene oxide and formaldehyde (also known as tyloxapol), poloxamers; and poloxamines. In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers; hydroxypropyl methylcellulose and hydroxypropyl methylcellulose ethers; carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers; or poloxamines.

Wetting agents suitable for the aqueous suspensions and dispersions described herein include, but are not limited to, cetyl alcohol, glycerol monostearate, polyoxyethylene sorbitan fatty acid esters (e.g., the commercially available Tweens® such as e.g., Tween 20® and Tween 80®, and polyethylene glycols, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphatidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, aspartame, chocolate, cinnamon, citrus, cocoa, cyclamate, dextrose, fructose, ginger, glycyrrhetinate, Glycyrrhiza (licorice) syrup, monoammonium glyrrhizinate (MagnaSweet®), malitol, mannitol, menthol, neohesperidine DC, neotame, Prosweet® Powder, saccharin, sorbitol, Stevia, sucralose, sucrose, sodium saccharin, saccharin, aspartame, acesulfame potassium, mannitol, sucralose, tagatose, thaumatin, vanilla, xylitol, or any combination thereof.

In some embodiments, an active agent is prepared as transdermal dosage form. In some embodiments, the transdermal formulations described herein include at least three components: (1) an active agent; (2) a penetration enhancer; and (3) an optional aqueous adjuvant. In some embodiments the transdermal formulations include additional components such as, but not limited to, gelling agents, creams and ointment bases, and the like. In some embodiments, the transdermal formulation is presented as a patch or a wound dressing. In some embodiments, the transdermal formulation further include a woven or non-woven backing material to enhance absorption and prevent the removal of the transdermal formulation from the skin. In other embodiments, the transdermal formulations described herein can maintain a saturated or supersaturated state to promote diffusion into the skin.

In one aspect, formulations suitable for transdermal administration of an active agent described herein employ transdermal delivery devices and transdermal delivery patches and can be lipophilic emulsions or buffered, aqueous solutions, dissolved and/or dispersed in a polymer or an adhesive. In one aspect, such patches are constructed for continuous, pulsatile, or on demand delivery of pharmaceutical agents. Still further, transdermal delivery of the active agents described herein can be accomplished by means of iontophoretic patches and the like. In one aspect, transdermal patches provide controlled delivery of an active agent. In one aspect, transdermal devices are in the form of a bandage comprising a backing member, a reservoir containing the active agent optionally with carriers, optionally a rate controlling barrier to deliver the active agent to the skin of the host at a controlled and predetermined rate over a prolonged period of time, and means to secure the device to the skin.

In further embodiments, topical formulations include gel formulations (e.g., gel patches which adhere to the skin). In some of such embodiments, a gel composition includes any polymer that forms a gel upon contact with the body (e.g., gel formulations comprising hyaluronic acid, pluronic polymers, poly(lactic-co-glycolic acid (PLGA)-based polymers or the like). In some forms of the compositions, the formulation comprises a low-melting wax such as, but not limited to, a mixture of fatty acid glycerides, optionally in combination with cocoa butter which is first melted. Optionally, the formulations further comprise a moisturizing agent.

In certain embodiments, delivery systems for pharmaceutical active agents may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, an active agent described herein may be administered topically and can be formulated into a variety of topically administrable compositions, such as solutions, suspensions, lotions, gels, pastes, medicated sticks, balms, creams or ointments. Such pharmaceutical active agents can contain solubilizers, stabilizers, tonicity enhancing agents, buffers and preservatives.

### Methods of Detection

### Methods of Detecting Genetic Risk Variants

In one aspect, provided are methods of evaluating a biological sample of genetic material for the presence, absence, and/or quantity of a nucleic acid sequence from a particular genetic risk variant or genotype of a subject. In some embodiments, the genetic risk variant, comprises a single nucleotide polymorphism (SNP) disclosed herein. A “genotype” refers to the genetic composition of an organism. A genotype may include one genetic risk variant, or a plurality of genetic risk variants. A genotype may also include one or more haplotypes.

In some embodiments, the SNP comprises a risk allele at rs4077515, a risk allele at rs10870077, a risk allele at rs2078178, a risk allele at rs795945, a risk allele at rs11053603, a risk allele at rs11053624, and/or a risk allele at rs16910631. In some embodiments, risk allele at rs4077515 comprises an “A”. In some embodiments, the risk allele at rs10870077 comprises a “C”. In some embodiments, the risk allele at rs2078178 comprises a “G” or an “A”. In some embodiments, the risk allele at rs16910631 comprises a “G”. In some instances, the genetic risk variant comprises any one of SEQ ID NOS: 1-7. In some instances, the genotype comprises one, two, three, four, five, six, or all seven of the genetic risk variants. In some instances, a haplotype is detected in a biological sample obtained from the subject. In some instances, the haplotype comprises rs2078178 and rs16910631, or rs4077515 and rs10870077. In some instances, the haplotype comprises SEQ ID NOS: 3 and 4, or SEQ ID NOS: 1 and 2.

In some cases, the nucleic acid sequence comprises deoxyribonucleic acid (DNA). In some instances, the nucleic acid sequence comprises a denatured DNA molecule or fragment thereof. In some instances, the nucleic acid sequence comprises DNA selected from: genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. The circular DNA may be cleaved or fragmented. In some instances, the nucleic acid sequence comprises ribonucleic acid (RNA). In some instances, the nucleic acid sequence comprises fragmented RNA. In some instances, the nucleic acid sequence comprises partially degraded RNA. In some instances, the nucleic acid sequence comprises a microRNA or portion thereof. In some instances, the nucleic acid sequence comprises an RNA molecule or a fragmented RNA molecule (RNA fragments) selected from: a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (IncRNA), a small nuclear RNA (snRNA), a circulating RNA, a cell-free RNA, an exosomal RNA, a vector-expressed RNA, an RNA transcript, a synthetic RNA, and combinations thereof.

Disclosed herein, in some embodiments, the genotype of the individual is determined by subjecting a sample obtained from the individual to a nucleic acid-based detection assay. In some instances, the nucleic acid-based detection assay comprises quantitative polymerase chain reaction (qPCR), gel electrophoresis (including for e.g., Northern or Southern blot), immunochemistry, in situ hybridization such as fluorescent in situ hybridization (FISH), cytochemistry, or sequencing. In some embodiments, the sequencing technique comprises next generation sequencing. In some embodiments, the methods involve a hybridization assay such as fluorogenic qPCR (e.g., TaqMan™ or SYBR green), which involves a nucleic acid amplification reaction with a specific primer pair, and hybridization of the amplified nucleic acid probes comprising a detectable moiety or molecule that is specific to a target nucleic acid sequence. An additional exemplary nucleic acid-based detection assay comprises the use of nucleic acid probes conjugated or otherwise immobilized on a bead, multi-well plate, or other substrate, wherein the nucleic acid probes are configured to hybridize with a target nucleic acid sequence. In some instances, the nucleic acid probe is specific to one or more genetic variants disclosed herein is used. In some instances, the nucleic acid probe specific to a SNP or SNV comprises a nucleic acid probe sequence sufficiently complementary to a risk or protective allele of interest, such that hybridization is specific to the risk or protective allele. In some instances, the nucleic acid probe specific to an indel comprises a nucleic acid probe sequence sufficiently complementary to an insertion of a nucleobase within a polynucleotide sequence flanking the insertion, such that hybridization is specific to the indel. In some instances, the nucleic acid probe specific to an indel comprises a probe sequence sufficiently complementary to a polynucleotide sequence flanking a deletion of a nucleobase within the polynucleotide sequence, such that hybridization is specific to the indel. The present disclosure provides exemplary probes that are hybridizable to a target nucleic acid sequence within rs4077515, rs10870077, rs2078178, rs795945, rs11053603, rs11053624, and rs16910631. The present disclosure provides exemplary probes provided in SEQ ID NOS: 15-21, respectively.

Disclosed herein, in some embodiments, are methods of detecting a genotype of an individual by subject a sample obtained from the individual to a nucleic acid amplification assay. In some instances, the amplification assay comprises polymerase chain reaction (PCR), qPCR, self-sustained sequence replication, transcriptional amplification system, Q-Beta Replicase, rolling circle replication, or any suitable other nucleic acid amplification technique. A suitable nucleic acid amplification technique is configured to amplify a region of a nucleic acid sequence comprising one or more genetic risk variants disclosed herein. In some instances, the amplification assays require primers. The nucleic acid sequence for the genetic risk variants and/or genes known or provided herein is sufficient to enable one of skill in the art to select primers to amplify any portion of the gene or genetic variants. A DNA sample suitable as a primer may be obtained, e.g., by polymerase chain reaction (PCR) amplification of genomic DNA, fragments of genomic DNA, fragments of genomic DNA ligated to adaptor sequences or cloned sequences. A person of skill in the art would utilize computer programs to design of primers with the desired specificity and optimal amplification properties, such as Oligo version 7.0 (National Biosciences). It will be apparent to one skilled in the art that controlled robotic systems are useful for isolating and amplifying nucleic acids and can be used.

In some embodiments, detecting the genotype of the subject comprises sequencing genetic material obtained from a biological sample from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modem sequencing technologies such as Illumina sequencing (e.g., Solexa), Roche 454 sequencing, Ion torrent sequencing, and SOLiD sequencing. In some cases, next-generation sequencing involves high-throughput sequencing methods. Additional sequencing methods available to one of skill in the art may also be employed.

In some instances, a number of nucleotides that are sequenced are at least 5, 10, 15, 20, 25, 30, 35, 40, 45, 50, 100, 150, 200, 300, 400, 500, 2000, 4000, 6000, 8000, 10000, 20000, 50000, 100000, or more than 100000 nucleotides. In some instances, the number of nucleotides sequenced is in a range of about 1 to about 100000 nucleotides, about 1 to about 10000 nucleotides, about 1 to about 1000 nucleotides, about 1 to about 500 nucleotides, about 1 to about 300 nucleotides, about 1 to about 200 nucleotides, about 1 to about 100 nucleotides, about 5 to about 100000 nucleotides, about 5 to about 10000 nucleotides, about 5 to about 1000 nucleotides, about 5 to about 500 nucleotides, about 5 to about 300 nucleotides, about 5 to about 200 nucleotides, about 5 to about 100 nucleotides, about 10 to about 100000 nucleotides, about 10 to about 10000 nucleotides, about 10 to about 1000 nucleotides, about 10 to about 500 nucleotides, about 10 to about 300 nucleotides, about 10 to about 200 nucleotides, about 10 to about 100 nucleotides, about 20 to about 100000 nucleotides, about 20 to about 10000 nucleotides, about 20 to about 1000 nucleotides, about 20 to about 500 nucleotides, about 20 to about 300 nucleotides, about 20 to about 200 nucleotides, about 20 to about 100 nucleotides, about 30 to about 100000 nucleotides, about 30 to about 10000 nucleotides, about 30 to about 1000 nucleotides, about 30 to about 500 nucleotides, about 30 to about 300 nucleotides, about 30 to about 200 nucleotides, about 30 to about 100 nucleotides, about 50 to about 100000 nucleotides, about 50 to about 10000 nucleotides, about 50 to about 1000 nucleotides, about 50 to about 500 nucleotides, about 50 to about 300 nucleotides, about 50 to about 200 nucleotides, or about 50 to about 100 nucleotides.

In some cases, a method provided herein comprises determining the presence, absence, and/or quantity of a nucleic acid sequence from a particular genetic risk variant. A non-limiting example of nucleic acid sequences that may be detected by the methods herein are provided in SEQ ID NOS: 8-14. In some cases, a portion of a nucleic acid sequence shown in SEQ ID NOS: 8-14 is detected, e.g., a sequence comprising at least about 10 contiguous nucleobases and comprising a nucleobase within [brackets] within SEQ ID NOS: 8-14. In some embodiments, provided is a method of detecting one or more genetic risk variants comprises detecting the presence, absence, and/or quantity of a nucleic acid sequence, or portion thereof, selected from SEQ ID NOS: 8-14, or a combination thereof. In some cases, a portion of a nucleic acid sequence provided herein comprises at least about 10, 15, 20, 25, 30, 35, 40, 45, or 50 contiguous nucleobases. In some cases, a portion of a nucleic acid sequence provided herein comprises between about 10 and about 50 contiguous nucleobases, between about 10 and about 40 contiguous nucleobases, between about 15 and about 50 contiguous nucleobases, between about 15 and about 40 contiguous nucleobases, between about 20 and about 50 contiguous nucleobases, and between about 20 and about 40 contiguous nucleobases. In some cases, a portion of a nucleic acid sequence provided herein comprises about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous nucleobases. In some cases, a portion of a nucleic acid sequence provided in SEQ ID NOS: 1-7 comprises a nucleobase within [brackets]. In some cases, a portion of a nucleic acid sequence comprising SEQ ID NO: 8 comprises an “A” allele at the bracketed position. In some cases, a portion of a nucleic acid sequence comprising SEQ ID NO: 9 comprises a “C” allele at the bracketed position. In some cases, a portion of a nucleic acid sequence comprising SEQ ID NO: 10 comprises an “G” or an “A” allele at the bracketed position. In some cases, a portion of a nucleic acid sequence comprising SEQ ID NO: 11 comprises a “G” allele at the bracketed position.

In some embodiments, the method comprises determining the presence or absence of a one or more genetic risk variants in a biological sample comprising genetic material from a subject, as determined by detecting the presence or absence of: a nucleic acid sequence at least or about 90% identical to SEQ ID NO: 8, a nucleic acid sequence at least or about 90% identical to SEQ ID NO: 9, a nucleic acid sequence at least or about 90% identical to a portion of SEQ ID NO: 10, a nucleic acid sequence at least or about 90% identical to a portion of SEQ ID NO: 11, a nucleic acid sequence at least or about 90% identical to SEQ ID NO: 12, a nucleic acid sequence at least or about 90% identical to SEQ ID NO: 13, a nucleic acid sequence at least or about 90% identical to SEQ ID NO: 14, or a combination thereof, in the genetic material. In some cases, if the one or more genetic risk variants is detected in the biological sample, the subject is administered a modulator of CARD9 and/or Dectin-1A, and/or an antimycotic agent.

In some embodiments, the method comprises determining the presence or absence of a one or more genetic risk variants in a biological sample comprising genetic material from a subject, as determined by detecting the presence or absence of: a nucleic acid sequence at least or about 95% identical to SEQ ID NO: 8, a nucleic acid sequence at least or about 95% identical to SEQ ID NO: 9, a nucleic acid sequence at least or about 95% identical to a portion of SEQ ID NO: 10, a nucleic acid sequence at least or about 95% identical to a portion of SEQ ID NO: 11, a nucleic acid sequence at least or about 95% identical to a portion of SEQ ID NO: 12, a nucleic acid sequence at least or about 95% identical to a portion of SEQ ID NO: 13, a nucleic acid sequence at least or about 95% identical to a portion of SEQ ID NO: 14, or a combination thereof, in the genetic material. In some cases, if the one or more genetic risk variants is detected in the biological sample, the subject is administered a modulator of CARD9 and/or Dectin-1A, and/or an antimycotic agent.

In some embodiments, the method comprises determining the presence or absence of a haplotype comprising rs4077515 and rs10870077 in a biological sample comprising of genetic material from a subject, as determined by detecting the presence or absence in the genetic material of: (a) SEQ ID NO: 1, SEQ ID NO: 2, a portion of SEQ ID NO: 1, a portion of SEQ ID NO: 2, or a combination thereof, and (b) SEQ ID NO: 8, SEQ ID NO: 9, a portion of SEQ ID NO: 8, a portion of SEQ ID NO: 9, or a combination thereof. In some cases, if the subject comprises rs4077515A and rs10870077C, the subject is administered a modulator of CARD9 and/or Dectin-1A, and/or an antimycotic agent. In some cases, if the subject is homozygous for rs4077515AA and rs10870077CC, the subject is administered a modulator of CARD9 and/or Dectin-1A, and/or an antimycotic agent.

In some embodiments, the method comprises determining the presence or absence of a haplotype comprising rs2078178 and rs16910631 in a biological sample comprising of genetic material from a subject, as determined by detecting the presence or absence in the genetic material of: (a) SEQ ID NO: 3, SEQ ID NO: 4, a portion of SEQ ID NO: 3, a portion of SEQ ID NO: 4, or a combination thereof, and (b) SEQ ID NO: 10, SEQ ID NO: 11, a portion of SEQ ID NO: 10, a portion of SEQ ID NO: 11, or a combination thereof. In some cases, if the subject comprises rs2078178G or rs2078178A and rs16910631G, the subject is administered a modulator of CARD9 and/or Dectin-1A, and/or an antimycotic agent. In some cases, if the subject is homozygous for rs2078178GG or rs2078178AA and rs16910631GG, the subject is administered a modulator of CARD9 and/or Dectin-1A, and/or an antimycotic agent.

In some instances, a method of detecting a genetic risk variant comprises contacting nucleic acids from a sample of a subject with a nucleic acid polymer that hybridizes to a region of a target nucleic acid sequence. In some cases, the target nucleic acid sequence is a sequence comprising at least about 30, 40, 50, 60, 70, 80, 90, 100, or all of SEQ ID NO: 1, wherein the target nucleic acid sequence comprises the risk allele. In some cases, the target nucleic acid sequence is a sequence comprising at least about 30, 40, 50, 60, 70, 80, 90, 100, or all of SEQ ID NO: 2, wherein the target nucleic acid sequence comprises the risk allele. In some cases, the target nucleic acid sequence is a sequence comprising at least about 30, 40, 50, 60, 70, 80, 90, 100, or all of SEQ ID NO: 3, wherein the target nucleic acid sequence comprises the risk allele. In some cases, the target nucleic acid sequence is a sequence comprising at least about 30, 40, 50, 60, 70, 80, 90, 100, or all of SEQ ID NO: 4, wherein the target nucleic acid sequence comprises the risk allele. In some cases, the target nucleic acid sequence is a sequence comprising at least about 30, 40, 50, 60, 70, 80, 90, 100, or all of SEQ ID NO: 5, wherein the target nucleic acid sequence comprises the risk allele. In some cases, the target nucleic acid sequence is a sequence comprising at least about 30, 40, 50, 60, 70, 80, 90, 100, or all of SEQ ID NO: 6, wherein the target nucleic acid sequence comprises the risk allele. In some cases, the target nucleic acid sequence is a sequence comprising at least about 30, 40, 50, 60, 70, 80, 90, 100, or all of SEQ ID NO: 7, wherein the target nucleic acid sequence comprises the risk allele. In some cases, the target nucleic acid sequence is a sequence comprising at least about 30, 40, 50, 60, 70, 80, 90, 100, or all of SEQ ID NO: 8, wherein the target nucleic acid sequence comprises the risk allele. In some cases, the method is a multiplex assay where two or more target nucleic acid sequences are detected. As a non-limiting example, the method comprises detecting the target nucleic acid sequence comprising the risk allele within SEQ ID NO: 1 and the target nucleic acid sequence comprising the risk allele within SEQ ID NO: 2. As another non-limiting example, the method comprises detecting the target nucleic acid sequence comprising the risk allele within SEQ ID NO: 3 and the target nucleic acid sequence comprising the risk allele within SEQ ID NO: 4.

The nucleic acid polymer can comprise an oligonucleotide of at least or about 5, 10, 15, 20, 25, 30, 35, 40, 45, 50, 75, 100 or more nucleobases in length and sufficient to specifically hybridize to a target nucleic acid sequence as described herein. In some instances, the nucleic acid polymer comprises between about 10 and about 100 nucleobases, between about 10 and about 75 nucleobases, between about 10 and about 50 nucleobases, between about 15 and about 100 nucleobases, between about 15 and about 75 nucleobases, between about 15 and about 50 nucleobases, between about 20 and about 100 nucleobases, between about 20 and about 75 nucleobases, between about 20 and about 50 nucleobases, between about 25 and about 100 nucleobases, between about 25 and about 75 nucleobases, or between about 25 and about 50 nucleobases. In some instances, the nucleic acid polymer hybridizes to a region of a target nucleic acid sequence of least one of SEQ ID NOS: 1-14. In some instances, the nucleic acid polymer hybridizes to a target nucleic acid sequence comprising SEQ ID NO: 1. In some instances, the nucleic acid polymer hybridizes to a target nucleic acid sequence comprising SEQ ID NO: 2. In some instances, the nucleic acid polymer hybridizes to a target nucleic acid sequence comprising SEQ ID NO: 3. In some instances, the nucleic acid polymer hybridizes to a target nucleic acid sequence comprising SEQ ID NO: 4. In some instances, the nucleic acid polymer hybridizes to a target nucleic acid sequence comprising SEQ ID NO: 5. In some instances, the nucleic acid polymer hybridizes to a target nucleic acid sequence comprising SEQ ID NO: 6. In some instances, the nucleic acid polymer hybridizes to a target nucleic acid sequence comprising SEQ ID NO: 7. In some instances, the nucleic acid polymer hybridizes to a target nucleic acid sequence comprising SEQ ID NO: 8. In some instances, the nucleic acid polymer hybridizes to a target nucleic acid sequence comprising SEQ ID NO: 9. In some instances, the nucleic acid polymer hybridizes to a target nucleic acid sequence comprising SEQ ID NO: 10. In some instances, the nucleic acid polymer hybridizes to a target nucleic acid sequence comprising SEQ ID NO: 11. In some instances, the nucleic acid polymer hybridizes to a target nucleic acid sequence comprising SEQ ID NO: 12. In some instances, the nucleic acid polymer hybridizes to a target nucleic acid sequence comprising SEQ ID NO: 13. In some instances, the nucleic acid polymer hybridizes to a target nucleic acid sequence comprising SEQ ID NO: 14. Hybridization may occur at standard hybridization temperatures, e.g., between about 35° C. and about 65° C. in a standard PCR buffer.

Further provided are primers useful for amplifying a nucleic acid of a target nucleic acid described herein. For example, for use in an amplification assay such as qPCR. In some instances, the primers hybridize to at least a portion of one of SEQ ID NOS: 1-14. In some instances, provided is a forward primer that hybridizes to at least about 10 contiguous bases of SEQ ID NO: 1, and a reverse primer that hybridizes to at least 10 contiguous bases of SEQ ID NO: 1, such that the forward and reverse primer flank the risk allele within SEQ ID NO: 1. In some instances, provided is a forward primer that hybridizes to at least about 10 contiguous bases of SEQ ID NO: 2, and a reverse primer that hybridizes to at least 10 contiguous bases of SEQ ID NO: 2, such that the forward and reverse primer flank the risk allele within SEQ ID NO: 2. In some instances, provided is a forward primer that hybridizes to at least about 10 contiguous bases of SEQ ID NO: 3, and a reverse primer that hybridizes to at least 10 contiguous bases of SEQ ID NO: 3, such that the forward and reverse primer flank nucleobase position 501 in SEQ ID NO: 3. In some instances, provided is a forward primer that hybridizes to at least about 10 contiguous bases of SEQ ID NO: 4, and a reverse primer that hybridizes to at least 10 contiguous bases of SEQ ID NO: 4, such that the forward and reverse primer flank nucleobase position 501 in SEQ ID NO: 4. In some instances, provided is a forward primer that hybridizes to at least about 10 contiguous bases of SEQ ID NO: 5, and a reverse primer that hybridizes to at least 10 contiguous bases of SEQ ID NO: 5, such that the forward and reverse primer flank nucleobase position 501 in SEQ ID NO: 5. In some instances, provided is a forward primer that hybridizes to at least about 10 contiguous bases of SEQ ID NO: 6, and a reverse primer that hybridizes to at least 10 contiguous bases of SEQ ID NO: 6, such that the forward and reverse primer flank nucleobase position 501 in SEQ ID NO: 6. In some instances, provided is a forward primer that hybridizes to at least about 10 contiguous bases of SEQ ID NO: 7, and a reverse primer that hybridizes to at least 10 contiguous bases of SEQ ID NO: 7, such that the forward and reverse primer flank nucleobase position 501 in SEQ ID NO: 7.

Further provided are probe or reporter sequences that hybridize to a target nucleic acid described herein. As a non-limiting example, a target nucleic acid of rs4077515, rs10870077, rs2078178, rs795945, rs11053603, rs11053624, and rs16910631. In some cases, the probes are reporters that comprise a dye label on one end and a quencher on the other end. When the probes are hybridized to a target nucleic acid, an added DNA polymerase may cleave those hybridized probes, separating the reporter dye from the quencher, and thus increasing fluorescence by the reporter. In some cases, provided is a probe comprising a nucleic acid polymer sequence described above herein. Non-limiting examples of probe sequences are provided in SEQ ID NOS: 15-21. The probes may be used to detect and/or quantify the presence of a target nucleic acid in a given sample.

In some instances, provided is a probe comprising SEQ ID NO: 15. In some instances, provided is a probe comprises at least 70%, 80%, 85%, 90%, 91%, 92%, 93%, 94%, 95%, 96%, 97%, 98% or 99% sequence identity to SEQ ID NO: 15. In some instances, provided is a probe comprising SEQ ID NO: 16. In some instances, provided is a probe comprises at least 70%, 80%, 85%, 90%, 91%, 92%, 93%, 94%, 95%, 96%, 97%, 98% or 99% sequence identity to SEQ ID NO: 16. In some instances, provided is a probe comprising SEQ ID NO: 17. In some instances, provided is a probe comprises at least 70%, 80%, 85%, 90%, 91%, 92%, 93%, 94%, 95%, 96%, 97%, 98% or 99% sequence identity to SEQ ID NO: 17. In some instances, provided is a probe comprising SEQ ID NO: 18. In some instances, provided is a probe comprises at least 70%, 80%, 85%, 90%, 91%, 92%, 93%, 94%, 95%, 96%, 97%, 98% or 99% sequence identity to SEQ ID NO: 18. In some instances, provided is a probe comprising SEQ ID NO: 19. In some instances, provided is a probe comprises at least 70%, 80%, 85%, 90%, 91%, 92%, 93%, 94%, 95%, 96%, 97%, 98% or 99% sequence identity to SEQ ID NO: 19. In some instances, provided is a probe comprising SEQ ID NO: 20. In some instances, provided is a probe comprises at least 70%, 80%, 85%, 90%, 91%, 92%, 93%, 94%, 95%, 96%, 97%, 98% or 99% sequence identity to SEQ ID NO: 20. In some instances, provided is a probe comprising SEQ ID NO: 21. In some instances, provided is a probe comprises at least 70%, 80%, 85%, 90%, 91%, 92%, 93%, 94%, 95%, 96%, 97%, 98% or 99% sequence identity to SEQ ID NO: 21.

Examples of molecules that are utilized as probes include, but are not limited to, RNA and DNA. In some embodiments, the term “probe” with regards to nucleic acids, refers to any molecule that is capable of selectively binding to a specifically intended target nucleic acid sequence. In some instances, probes are specifically designed to be labeled, for example, with a radioactive label, a fluorescent label, an enzyme, a chemiluminescent tag, a colorimetric tag, or other labels or tags that are known in the art. In some instances, the fluorescent label comprises a fluorophore. In some instances, the fluorophore is an aromatic or heteroaromatic compound. In some instances, the fluorophore is a pyrene, anthracene, naphthalene, acridine, stilbene, benzoxaazole, indole, benzindole, oxazole, thiazole, benzothiazole, canine, carbocyanine, salicylate, anthranilate, xanthenes dye, coumarin. Exemplary xanthene dyes include, e.g., fluorescein and rhodamine dyes. Fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent probes also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-1-sulfonic acid (EDANS). Exemplary coumarins include, e.g., 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine 3 (Cy3), indodicarbocyanine 5 (Cy5), indodicarbocyanine 5.5 (Cy5.5), 3-(-carboxy-pentyl)-3′-ethyl-5,5′-dimethyloxacarbocyanine (CyA); 1H, 5H, 11H, 15H-Xantheno[2,3, 4-ij: 5,6, 7-‘j’]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3, 6,7, 12,13, 16,17-octahydro-inner salt (TR or Texas Red); or BODIPY™ dyes. In some cases, the probe comprises FAM as the dye label.

In some instances, primers and/or probes described herein for detecting a target nucleic acid are used in an amplification reaction. In some instances, the amplification reaction is qPCR. An exemplary qPCR is a method employing a TaqMan™ assay.

In some instances, qPCR comprises using an intercalating dye. Examples of intercalating dyes include SYBR green I, SYBR green II, SYBR gold, ethidium bromide, methylene blue, Pyronin Y, DAPI, acridine orange, Blue View or phycoerythrin. In some instances, the intercalating dye is SYBR.

In some instances, a number of amplification cycles for detecting a target nucleic acid in an amplification assay is about 5 to about 30 cycles. In some instances, the number of amplification cycles for detecting a target nucleic acid is at least about 5 cycles. In some instances, the number of amplification cycles for detecting a target nucleic acid is at most about 30 cycles. In some instances, the number of amplification cycles for detecting a target nucleic acid is about 5 to about 10, about 5 to about 15, about 5 to about 20, about 5 to about 25, about 5 to about 30, about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 cycles.

In one aspect, the methods provided herein for determining the presence, absence, and/or quantity of a nucleic acid sequence from a particular genotype comprise an amplification reaction such as qPCR. In an exemplary method, genetic material is obtained from a sample of a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In an exemplary embodiment, the nucleic acid material is extracted in water. In some cases, extraction does not comprise nucleic acid purification.

In the exemplary qPCR assay, the nucleic acid sample is combined with primers and probes specific for a target nucleic acid that may or may not be present in the sample, and a DNA polymerase. An amplification reaction is performed with a thermal cycler that heats and cools the sample for nucleic acid amplification, and illuminates the sample at a specific wavelength to excite a fluorophore on the probe and detect the emitted fluorescence. For TaqMan™ methods, the probe may be a hydrolysable probe comprising a fluorophore and quencher that is hydrolyzed by DNA polymerase when hybridized to a target nucleic acid. In some cases, the presence of a target nucleic acid is determined when the number of amplification cycles to reach a threshold value is less than 30, 29, 28, 27, 26, 25, 24, 23, 22, 21, or 20 cycles.

Methods of Detecting and Quantifying Soluble CARD9 and/or Dectin-1A

In one aspect, provided herein are methods of analyzing CARD9 and/or Dectin-1A protein levels in a subject by detecting and quantifying said levels from a biological sample of the subject. Non-limiting examples of biological sample materials include serum, plasma, fecal matter, and/or whole blood. CARD9 and/or Dectin-1A may be detected by use of an antibody-based assay, where an anti-CARD9 or an anti-Dectin-1A antibody is utilized. For antibody-based detection methods, the anti-CARD9 antibody may bind to any region of CARD9. An exemplary method of analysis comprises performing an enzyme-linked immunosorbent assay (ELISA). The ELISA assay may be a sandwich ELISA or a direct ELISA. Another exemplary method of analysis comprises a single molecule array, e.g., Simoa. Other exemplary methods of detection include immunohistochemistry and lateral flow assay.

In some cases, CARD9 and/or Dectin-1A protein may be detected by detecting binding between CARD9 and/or Dectin-1A and binding partners of CARD9 and/or Dectin-1A. Non-limiting examples of binding partners of CARD9 include Tripartite Motif Containing 62 (TRIM62), B Cell CLL/Lymphoma 10 (BCL10), and Rubicon. Non-limiting examples of binding partners of Dectin-1A include beta-1,3 glucan, and CARD9. Methods of analysis of binding between CARD9 and/or Dectin-1A and CARD9 and/or Dectin-1A binding partners comprise performing an assay in vivo or in vitro, or ex vivo. In some instances, the assay may comprise co-immunoprecipitation (co-IP), pull-down, crosslinking protein interaction analysis, labeled transfer protein interaction analysis, or Far-western blot analysis, FRET based assay, including, for example FRET-FLIM, a yeast two-hybrid assay, BiFC, or split luciferase assay.

### Methods of Detecting Fungi on Intestinal Mucosal Surfaces

Aspects disclosed herein provide for the detection of fungal organisms in the biological sample form the subject. A prevalent target for evaluating fungal taxonomic relationships is the internal transcribed spacer (ITS) region, which is an area of non-functional RNA placed between ribosomal RNAs. The ITS region has been recognized as the official fungal barcode and is commonly used as a molecular marker in fungal studies due to the large number of copies per cell and deposited sequences in international databases. Fungal diversity can be determined by sequencing the ITS regions of ribosomal RNA derived from fungi. Various molecular methodologies such as sanger and 454 sequencing technologies have provided new insight into detecting fungal species in the samples.

In various embodiments, the detection of fungal organisms in the biological sample is done by sequencing the internal transcribed spacer (ITS) regions. In some embodiments, the primers used are general primers for ITS regions. In various other embodiments, the ITS primers used are specific to fungal organisms ITS region. In various embodiments, the primers are ITS1F and ITS2, as described herein. In various other embodiments, the ITS region is amplified, cloned and sequenced. In various embodiments, fungal ITS libraries are generated and analyzed. In some embodiments, the biological samples comprise a portion of the intestine. Portions of the intestines comprise, but are not limited to, the colon, cecum, small intestine and duodenum. In various other embodiments, the biological sample comprises the mucosal surfaces of the intestine.

Sequencing of highly variable regions of ribosomal DNA has become a standard method for identifying microbial species. Specific regions of ribosomal genes containing structural parts of ribosomal RNAs are highly conserved across species, making it possible to design generic primers to amplify ribosomal DNA from broad classes of related organisms (such as “bacteria” or “fungi”). Sequencing the highly variable internal parts of these amplified fragments permits accurate identification of the organisms. The intestinal bacterial microbiome has been frequently assessed by this method. For example, one recent application of this method identified over 5600 types of bacteria in the human gut. The method had not been applied to the mouse intestinal fungal microbiome, until this study. A small number of mostly environmental fungal communities had been previously analyzed using Roche 454 pyrosequencing, an established technology that provides relatively long reads (^{≈} 3-400 bases). The inventors have focused on moving the approach to Illumina Nex-gen sequencing, a newer technology that provides orders of magnitude more data, but in the form of shorter reads (^{≈} 100-150 bases).

In various embodiments, fecal specimens are collected from the DSS-treated or untreated wild-type, Card9 knockout, Dectin-1 knockout and Dectin-2 knockout mice. In some embodiments, the fecal specimens are collected in the form of a biological sample comprising a stool sample. In other embodiments, human fecal specimens are collected from a human subject with an inflammatory disease or condition, such as those disclosed herein. In some embodiments, the fecal specimens are collected in the form of a biological sample comprising a stool sample. In some embodiments, the fecal specimens are collected using a water-gavage during a colonoscopy procedure. In some embodiments, the fecal specimens comprise one or more fungal organisms. In various embodiments, the fecal specimens are analyzed using the sequencing methodologies disclosed herein. In a non-limiting example, ITS-2 (internal transcribed spacer-2) region of fungal ribosomal DNA is amplified. In various embodiments, rDNA regions (i.e., 18S) are assessed. In some embodiments, the ITS-2 sequences are identified via Roche 454 pyrosequencing over. In some embodiments, the samples are assessed via Nex-gen sequencing and/or 454 pyrosequencing.

### Methods of Detecting Serological Markers

Aspects disclosed herein provide for the detection of serological markers in the biological sample. In various embodiments, the serological marker detected comprise, but are not limited to, anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globosa antibody, anti-Cladosporium spp. antibody, or a combination thereof.

In some embodiments, the serological marker is an enzyme of a fungus; for example, a lipase. In particular embodiments, the serological marker is Malassezia lipase 1, or Cladosporium lipase 1.

In other embodiments, the serological marker is detected using an immunoassay. In yet other embodiments, the immunoassay comprises, but is not limited to, Radioimmunoassay (RIA), Counting Immunoassay (CIA), Enzyme Immunoassays (EIA) or Enzyme-linked immunosorbent assays (ELISA), Fluoroimmnoassay (FIA), single-molecule detection (Simoa), and Chemiluminescenceimmunoassay (CLIA). In various embodiments, the immunoassay is Simoa.

### Compositions for Detection

Aspects disclosed herein provide for a composition comprising a polynucleotide sequence comprising at least 10 but less than 50 contiguous nucleotides of any one of SEQ ID NO: 1 (rs4077515), SEQ ID NO: 2 (rs10870077), SEQ ID NO: 3 (rs2078178), and SEQ ID NO: 4 (rs16910631), SEQ ID NO: 5 (rs7959451), SEQ ID NO: 6 rs11053603), and SEQ ID NO: 7 (rs11053624), or reverse complements thereof, wherein the contiguous polynucleotide sequence comprises a detectable molecule. In various embodiments, the detectable molecule comprises a fluorophore. In other embodiments, the polynucleotide sequences further comprise a quencher. In some embodiments, the contiguous polynucleotide sequence comprises at least 10 but less than 50 contiguous nucleotides of any one of SEQ ID NOS: 1-7, including the risk allele.

Aspects disclosed herein provide for a composition comprising an antibody or antigen-binding fragment that specifically binds to CARD9 and/or Dectin-1A, wherein the antibody or antigen-binding fragment comprises a detectable molecule. In various embodiments, the antibody comprises a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, or a combination thereof. In some embodiments, the antibody or antigen-binding fragment comprises an IgG antibody, an IgM antibody, and/or an IgE antibody. In some embodiments, the detectable molecule comprises a fluorophore.

Various embodiments provide for a composition comprising an antibody or antigen-binding fragment that specifically binds to Malassezia spp. or Cladosporium spp., or isolated polypeptides therefrom, wherein the antibody or antigen-binding fragment comprises a detectable molecule. Also provided is a composition comprising an antibody or antigen-binding fragment that specifically binds to an anti-Malassezia spp. antibody or and anti-Cladosporium spp. antibody. In some embodiments, the Malassezia spp. comprises M. restricta, M. pachydermatis, M. furfur, and/or M. globosa. In particular embodiments, the Malassezia spp comprises M. globosa. In some embodiments, the antibody comprises a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, or a combination thereof. In some embodiments, the antibody or antigen-binding fragment comprises an IgG antibody, an IgM antibody and/or an IgE antibody. In other embodiments, the detectable molecule comprises a fluorophore.

Various embodiments provide for a method of detecting one or more genetic risk variants in a subject suffering from a disease or condition, comprising contacting a biological sample comprising deoxyribonucleic acid (DNA) obtained from a subject with a composition for detection, as described herein, under conditions configured to hybridize the composition to the DNA from the subject and detecting a presence or absence of a hybridization product comprising the DNA obtained from the subject and the composition by detecting the detectable molecule, wherein the presence of the hybridization product indicates a presence of one or more genetic risk variants in the subject.

Various embodiments provide for a method of detecting a level of CARD9 expression in a subject suffering from a disease or condition, the method comprising contacting a biological sample obtained from a subject with the composition described herein, under conditions configured to bind the composition to the CARD9 and detecting a presence or absence of the detectable molecule indicative of binding between the CARD9 and the composition.

Various embodiments provide for a method of detecting one or more serological markers in a subject suffering from a disease or condition, the method comprising contacting a biological sample obtained from a subject with the composition described herein, under conditions configured to bind the composition to the Malassezia spp. or Cladosporium spp., or the isolated polypeptides therefrom and detecting a presence or absence of the detectable molecule indicative of binding between the Malassezia spp. (e.g., M. globosa.) or Cladosporium spp., or the isolated polypeptides therefrom, and the composition.

Various embodiments provide for a method of detecting one or more genetic risk variants in a subject suffering from a disease or condition, the method comprising (a) contacting a biological sample comprising deoxyribonucleic acid (DNA) obtained from a subject suffering from a disease or condition with a composition sufficiently complementary to, and capable of, hybridizing to the one or more genetic risk variants, the composition comprising, (i) a labeled polynucleotide probe comprising SEQ ID NO: 1, or a reverse complement thereof, (ii) a labeled polynucleotide probe comprising SEQ ID NO: 2, or a reverse complement thereof, (iii) a labeled polynucleotide probe comprising SEQ ID NO: 3, or a reverse complement thereof, (iv) a labeled polynucleotide probe comprising SEQ ID NO: 4, or a reverse complement thereof, (v) a labeled polynucleotide probe comprising SEQ ID NO: 5, or a reverse complement thereof, (vi) a labeled polynucleotide probe comprising SEQ ID NO: 6, or a reverse complement thereof, (vii) a labeled polynucleotide probe comprising SEQ ID NO: 7, or a reverse complement thereof, (viii) a labeled polynucleotide probe comprising a nucleic acid sequence that differs from a probe selected from the group consisting of (i)-(vii) by up to three nucleobases, provided the detectably labeled polynucleotide probe hybridizes to the one or more genetic risk variants, (ix) a labeled polynucleotide probe comprising a nucleic acid sequence complementary to a probe selected from the group consisting of (i)-(viii) or (x) a combination of probes selected from the group consisting of (i)-(ix) and (b) detecting a presence or an absence of a hybridization product comprising the DNA obtained from the subject and the composition, by detecting the labeled polynucleotide probe. In some embodiments, the labeled polynucleotide probe further comprises a fluorophore. In other embodiments, the labeled polynucleotide probe further comprises a quencher.

### Kits

The disclosure also provides kits for detecting the presence, absence, and/or quantity of a target nucleic acid described herein. In some instances, kits are provided for detecting and/or quantifying a nucleic acid sequence for a genetic risk variant, haplotype and/or fungal DNA. In some embodiments, the genetic variants and/or haplotypes are at gene loci comprising CLEC7A and/or CARD9. In various embodiments, the one or more genetic risk variants comprises a risk allele at rs4077515, a risk allele at rs10870077, a risk allele at rs2078178, a risk allele at rs7959451, a risk allele at rs11053603, a risk allele at rs11053624, and/or a risk allele at rs16910631. In some embodiments, the haplotype comprises a haplotype at the CLEC7A and/or CARD9 gene loci. In some embodiments, the haplotype comprises rs2078178 and rs16910631. In some embodiments, the haplotype comprises rs4077515 and rs10870077. In some embodiments, the kit further comprises reagents for detecting the presence, absence, and/or quantity of a serological marker described herein.

The disclosure provides kits for detecting the presence, absence, and/or quantity of a serological marker described herein. In some instances, the serological markers comprises anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globosa antibody, anti-Cladosporium spp. antibody, or a combination thereof. In some embodiments, the serological marker is an enzyme of a fungus; for example, a lipase. In particular embodiments, the serological marker is Malassezia lipase 1, or Cladosporium lipase 1.

In some instances, the kit comprises an antibody specific to one or more of the serological markers. In some instances, the antibody or antigen-binding fragment further comprises a paramagnetic particle. In some instances, the kit comprises a composition for detection described herein. In some embodiments, the kit further comprises reagents for detecting and/or quantifying a nucleic acid sequence for a genetic risk variant, haplotype and/or fungal DNA.

In some embodiments, the kit includes nucleic acid or polypeptide isolation reagents. In some embodiments, the kit includes one or more detection reagents, for example probes and/or primers for amplification of, or hybridization to, a target nucleic acid sequence. In some embodiments, the target nucleic acid sequence comprises CLEC7A and/or CARD9. In some embodiments, the target nucleic acid sequence comprises a genetic risk variant comprising a risk allele at rs4077515, a risk allele at rs10870077, a risk allele at rs2078178, a risk allele at rs7959451, a risk allele at rs11053603, a risk allele at rs11053624, and/or a risk allele at rs16910631. In some embodiments, the target nuclei acid sequence comprises fungal DNA. In some embodiments, the probe comprises a nucleic acid sequence comprising one or more of SEQ ID NOS: 15-21. In some instances, the target nucleic acid is associated with a disease or condition comprising an inflammatory disease, fibrostenotic disease, and/or fibrotic disease. In some embodiments, the kit includes additional primers and probes for control genes, such as housekeeping genes. In some embodiments, the primers and probes for control genes are used, for example, in ΔCₜ calculations. In some embodiments, the probes or primers are labeled with an enzymatic, florescent, or radionuclide label.

In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic.

In some embodiments, a kit includes one or more additional containers, each with one or more of various materials (such as reagents, optionally in concentrated form, and/or devices) desirable from a commercial and user standpoint for use of described herein. Non-limiting examples of such materials include, but not limited to, buffers, primers, enzymes, diluents, filters, carrier, package, container, vial and/or tube labels listing contents and/or instructions for use and package inserts with instructions for use. A set of instructions is optionally included. In a further embodiment, a label is on or associated with the container. In yet a further embodiment, a label is on a container when letters, numbers or other characters forming the label are attached, molded or etched into the container itself, a label is associated with a container when it is present within a receptacle or carrier that also holds the container, e.g., as a package insert. In other embodiments a label is used to indicate that the contents are to be used for a specific therapeutic application. In yet another embodiment, a label also indicates directions for use of the contents, such as in the methods described herein.
1. A method of treating a disease or condition in a subject comprising administering to the subject a therapeutically effective amount of active agent, provided one or more genetic risk variants and/or biomarkers is detected in a biological sample obtained from the subject.
2. The method of paragraph 1, wherein the one or more genetic risk variants and/or biomarkers is detected using polymerase chain reaction (PCR), reverse-transcription polymerase chain reaction (RT-PCR), a genotyping array, automated sequencing, or a combination thereof.
3. The method of paragraph 1, wherein the one or more genetic risk variants comprise single nucleotide variants within the caspase recruitment domain family member 9 (CARD9) gene locus.
4. The method of paragraph 1, wherein the one or more genetic risk variants comprise single nucleotide variants within the c-type lectin domain containing 7A (CLEC7A) gene locus.
5. The method of paragraph 1, wherein the one or more genetic risk variants comprises one copy (heterozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
6. The method of paragraph 1, wherein the one or more genetic risk variants comprises two copies (homozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
7. The method of paragraph 6, wherein the risk allele at rs4077515 comprises an “A”, the risk allele at rs10870077 comprises a “C”, the risk allele at rs2078178 comprises a “G” or an “A”, and the risk allele at rs16910631 comprises a “G”.
8. The method of paragraph 1, wherein the one or more genetic risk variants and/or biomarkers is associated with a risk that the subject has, or will develop, intestinal mycobiota characterized by an increase in Malassezia spp. or Cladosporium spp.
9. The method of paragraph 1, wherein the disease or condition comprises inflammatory bowel disease, Crohn's disease, or ulcerative colitis.
10. The method of paragraph 9, wherein the ulcerative colitis comprises medically refractory ulcerative colitis.
11. The method of paragraph 10, wherein the Crohn's disease comprises medically refractory Crohn's disease.
12. The method of paragraph 1, wherein the subject is a mammal.
13. The method of paragraph 1, wherein the subject is a human.
14. The method of paragraph 1, wherein the biological sample comprises tissue biopsy, whole blood, plasma, serum, fecal specimen, urine, or saliva.
15. The method of paragraph 1, wherein active agent comprises an antimycotic agent, an inhibitor of caspase recruitment domain family member 9 (CARD9) activity or expression, or a modulator of dendritic cell-associated c-type lectin-1 (Dectin-1A) activity or expression, or a combination thereof.
16. The method of paragraph 15, wherein the antimycotic agent comprises a fungicide agent or a fungistatic agent.
17. The method of paragraph 16, wherein the antimycotic agent comprises a polyene, an azole, an echinocandin, an flucytosine, an allylamine, a tolnaftate, or griseofulvin.
18. The method of paragraph 17, wherein the azole comprises triazole, imidazole, clotrimazole, ketoconazole, itraconazole, terconazole, oxiconazole, miconazole, econazole, tioconazole, voriconazole, fluconazole, isavuconazole, itraconazole, pramiconazole, ravuconazole, or posaconazole.
19. The method of paragraph 17, wherein the polyene comprises amphotericin B, nystatin, or natamycin.
20. The method of paragraph 17, wherein the echinocandin comprises caspofungin, anidulafungin, or micafungin.
21. The method of paragraph 17, wherein the allylamine comprises naftifine or terbinafine.
22. The method of paragraph 15, wherein the inhibitor of CARD9 activity or expression comprises an antibody, a small molecule, a direct inhibitor of CARD9, an indirect inhibitor of CARD9, an allosteric modulator of CARD9, an anti-CARD9 antibody or antibody fragment, antibody or antibody fragment that specifically binds to Rubicon, an anti-Tripartite Motif Containing 62 (TRIM62) antibody or antibody fragment, an antibody or antibody fragment that specifically binds to B Cell CLL/Lymphoma 10 (BCL10), an inhibitor of CARD9-Rubicon interaction, an inhibitor of CARD9-Tripartite Motif Containing 62 (TRIM62) interaction, an inhibitor of CARD9-B Cell CLL/Lymphoma 10 (BCL10) interaction, a small molecule that specifically binds CARD9, a small molecule that specifically binds to Rubicon, a small molecule that specifically binds to Tripartite Motif Containing 62 (TRIM62), a small molecule that specifically binds to B Cell CLL/Lymphoma 10 (BCL10), an inhibitor of CARD9-Rubicon interaction, an inhibitor of CARD9-Tripartite Motif Containing 62 (TRIM62) interaction, an inhibitor of B Cell CLL/Lymphoma 10 (BCL10)-CARD9 interaction or a combination thereof.
23. The method of paragraph 15, wherein the modulator of dendritic cell-associated c-type lectin-1 (Dectin-1A) activity or expression comprises an antibody or antibody fragment, a small molecule, an allosteric modulator, an agonist, an antagonist, a direct modulator of Dectin-1A, an indirect modulator of Dectin-1A.
24. The method of paragraph 1, wherein a presence or a level of one or more serological markers is detected in the biological sample obtained from the subject.
25. The method of paragraph 24, wherein the one or more serological markers comprises anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globosa antibody, anti-Cladosporium spp. antibody, or a combination thereof.
26. The method of paragraph 24, wherein the presence or the level of the one or more serological markers is detected using an enzyme-linked immunosorbent assay (ELISA), a single molecule array (Simoa), immunohistochemistry, internal transcribed spacer (ITS) sequencing, or any combination thereof.
27. A method of treating a disease or condition in a subject comprising administering to the subject a therapeutically effective amount of an active agent, provided one or more serological markers is detected in a biological sample obtained from the subject.
28. The method of paragraph 27, wherein the one or more serological markers is detected using an enzyme-linked immunosorbent assay (ELISA), a single molecule array (Simoa), immunohistochemistry, internal transcribed spacer (ITS) sequencing, or any combination thereof.
29. A method of treating a disease or condition in a subject comprising administering to the subject a therapeutically effective amount of an active agent, provided an elevated level of one or more serological markers is detected in a biological sample obtained from the subject, as compared to a level of the one or more serological markers in an individual who does not have the disease or condition.
30. The method of paragraph 29, wherein the elevated level of the one or more serological markers is detected using an enzyme-linked immunosorbent assay (ELISA), a single molecule array (Simoa), immunohistochemistry, internal transcribed spacer (ITS) sequencing, or any combination thereof.
31. The method of paragraphs 29 or 30, wherein the one or more serological markers comprises anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globosa antibody, anti-Cladosporium spp. antibody, or a combination thereof.
32. The method of paragraph 29, wherein the disease or condition comprises inflammatory bowel disease, Crohn's disease, or ulcerative colitis.
33. The method of paragraph 32, wherein the ulcerative colitis comprises medically refractory ulcerative colitis.
34. The method of paragraph 32, wherein the Crohn's disease comprises medically refractory Crohn's disease.
35. The method of paragraph 29, wherein the subject is a mammal.
36. The method of paragraph 29, wherein the subject is a human.
37. The method of paragraph 29, wherein the biological sample comprises tissue biopsy, whole blood, plasma, serum, fecal specimen, urine, or saliva.
38. The method of paragraph 29, wherein active agent comprises an antimycotic agent, an inhibitor of caspase recruitment domain family member 9 (CARD9) activity or expression, or a modulator of dendritic cell-associated c-type lectin-1 (Dectin-1A) activity or expression, or a combination thereof.
39. The method of paragraph 38, wherein the antimycotic agent comprises a fungicide agent or a fungistatic agent.
40. The method of paragraph 38, wherein the antimycotic agent comprises a polyene, an azole, an echinocandin, an flucytosine, an allylamine, a tolnaftate, or griseofulvin.
41. The method of paragraph 40, wherein the azole comprises triazole, imidazole, clotrimazole, ketoconazole, itraconazole, terconazole, oxiconazole, miconazole, econazole, tioconazole, voriconazole, fluconazole, isavuconazole, itraconazole, pramiconazole, ravuconazole, or posaconazole.
42. The method of paragraph 40, wherein the polyene comprises amphotericin B, nystatin, or natamycin.
43. The method of paragraph 40, wherein the echinocandin comprises caspofungin, anidulafungin, or micafungin.
44. The method of paragraph 40, wherein the allylamine comprises naftifine or terbinafine.
45. The method of paragraph 38, wherein the inhibitor of CARD9 activity or expression comprises an antibody, a small molecule, a direct inhibitor of CARD9, an indirect inhibitor of CARD9, an allosteric modulator of CARD9, an anti-CARD9 antibody or antibody fragment, antibody or antibody fragment that specifically binds to Rubicon, an anti-Tripartite Motif Containing 62 (TRIM62) antibody or antibody fragment, an antibody or antibody fragment that specifically binds to B Cell CLL/Lymphoma 10 (BCL10), an inhibitor of CARD9-Rubicon interaction, an inhibitor of CARD9-Tripartite Motif Containing 62 (TRIM62) interaction, an inhibitor of CARD9-B Cell CLL/Lymphoma 10 (BCL10) interaction, a small molecule that specifically binds CARD9, a small molecule that specifically binds to Rubicon, a small molecule that specifically binds to Tripartite Motif Containing 62 (TRIM62), a small molecule that specifically binds to B Cell CLL/Lymphoma 10 (BCL10), an inhibitor of CARD9-Rubicon interaction, an inhibitor of CARD9-Tripartite Motif Containing 62 (TRIM62) interaction, an inhibitor of B Cell CLL/Lymphoma 10 (BCL10)-CARD9 interaction or a combination thereof.
46. The method of paragraph 38, wherein the modulator of dendritic cell-associated c-type lectin-1 (Dectin-1A) activity or expression comprises an antibody or antibody fragment, a small molecule, an allosteric modulator, an agonist, an antagonist, a direct modulator of Dectin-1A, an indirect modulator of Dectin-1A.
47. The method of paragraph 38, wherein one or more genetic risk variants and/or biomarkers is detected in the biological sample obtained from the subject.
48. The method of paragraph 47, wherein the one or more genetic risk variants and/or biomarkers is associated with a risk that the subject has, or will develop, intestinal mycobiota characterized by an increase in Malassezia spp. or Cladosporium spp.
49. The method of paragraph 47, wherein the one or more genetic risk variants comprise single nucleotide variants within the caspase recruitment domain family member 9 (CARD9) gene locus.
50. The method of paragraph 47, wherein the one or more genetic risk variants comprise single nucleotide variants within the c-type lectin domain containing 7A (CLEC7A) gene locus.
51. The method of paragraph 47, wherein the one or more genetic risk variants comprises one copy (heterozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
52. The method of paragraph 47, wherein the one or more genetic risk variants comprises two copies (homozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
53. The method of paragraph 47, wherein the risk allele at rs4077515 comprises an “A”, the risk allele at rs10870077 comprises a “C”, the risk allele at rs2078178 comprises a “G” or an “A”, and the risk allele at rs16910631 comprises a “G”.
54. The method of paragraph 47, wherein the one or more genetic risk variants and/or biomarkers is detected using polymerase chain reaction (PCR), reverse-transcription polymerase chain reaction (RT-PCR), a genotyping array, automated sequencing, or a combination thereof.
55. A method of treating a disease or condition in a subject comprising administering to the subject a therapeutically effective amount of an inhibitor of caspase recruitment domain family member 9 (CARD9) activity or expression, provided one or more genetic risk variants and/or biomarkers is detected in a biological sample obtained from the subject.
56. The method of paragraph 55, wherein the one or more genetic risk variants and/or biomarkers is detected using polymerase chain reaction (PCR), reverse-transcription polymerase chain reaction (RT-PCR), a genotyping array, automated sequencing, or a combination thereof.
57. The method of paragraph 55, wherein a presence or a level of one or more serological markers is detected in the biological sample obtained from the subject.
58. The method of paragraph 57, wherein the one or more serological markers comprises anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globosa antibody, anti-Cladosporium spp. antibody, or a combination thereof.
59. The method of paragraph 57 wherein the presence or the level of the one or more serological markers is detected using an enzyme-linked immunosorbent assay (ELISA), a single molecule array (Simoa), immunohistochemistry, internal transcribed spacer (ITS) sequencing, or any combination thereof.
60. A method of inhibiting or reducing caspase recruitment domain family member 9 (CARD9) activity or expression in a subject suffering from a disease or condition, comprising:
   subjecting a biological sample obtained from a subject suffering from a disease or condition to an assay suitable to detect one or more genetic risk variants and/or biomarkers; and
   administering to the subject a therapeutically effective amount of an inhibitor of caspase recruitment domain family member 9 (CARD9), provided the one or more genetic risk variants and/or biomarkers is detected in the biological sample obtained from the subject.
61. The method of paragraph 60, wherein the assay suitable to detect the one or more genetic risk variants and/or biomarkers comprises polymerase chain reaction (PCR), reverse-transcription polymerase chain reaction (RT-PCR), a genotyping array, automated sequencing, or a combination thereof.
62. The method of paragraph 60, further comprising:
   subjecting a biological sample obtained from a subject suffering from a disease or condition to an assay suitable to detect a presence or a level of one or more serological markers; and
   administering to the subject the therapeutically effective amount of the inhibitor of CARD9, provided the presence or the level of the one or more serological markers is detected in the biological sample obtained from the subject.
63. The method of paragraph 62, wherein the one or more serological markers comprises anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globosa antibody, anti-Cladosporium spp. antibody, or a combination thereof.
64. The method of paragraph 62, wherein the assay suitable to detect the presence or the level of the one or more serological markers comprises enzyme-linked immunosorbent assay (ELISA), a single molecule array (Simoa), immunohistochemistry, internal transcribed spacer (ITS) sequencing, or any combination thereof.
65. The method of paragraphs 55 or 60, wherein the one or more genetic risk variants comprise single nucleotide variants within the caspase recruitment domain family member 9 (CARD9) gene locus.
66. The method of paragraphs 55 or 60, wherein the one or more genetic risk variants comprise single nucleotide variants within the c-type lectin domain containing 7A (CLEC7A) gene locus.
67. The method of paragraph 55 or 60, wherein the one or more genetic risk variants comprises one copy (heterozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
68. The method of paragraphs 55 or 60, wherein the one or more genetic risk variants comprises two copies (homozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
69. The method of paragraph 68, wherein the risk allele at rs4077515 comprises an “A”, the risk allele at rs10870077 comprises a “C”, the risk allele at rs2078178 comprises a “G” or an “A”, and the risk allele at rs16910631 comprises a “G”.
70. The method of paragraphs 55 or 60, wherein the one or more genetic risk variants and/or biomarkers is associated with a risk that the subject has, or will develop, intestinal mycobiota characterized by an increase in Malassezia spp. or Cladosporium spp.
71. The method of paragraphs 55 or 60, wherein the disease or condition comprises inflammatory bowel disease, Crohn's disease, or ulcerative colitis.
72. The method of paragraph 71, wherein the ulcerative colitis comprises medically refractory ulcerative colitis.
73. The method of paragraph 71, wherein the Crohn's disease comprises medically refractory Crohn's disease.
74. The method of paragraphs 55 or 60, wherein the subject is a mammal.
75. The method of paragraphs 55 or 60, wherein the subject is a human.
76. The method of paragraphs 55 or 60, wherein the biological sample comprises tissue biopsy, whole blood, plasma, serum, fecal specimen, urine, or saliva.
77. The method of paragraphs 55 or 60, wherein the inhibitor of CARD9 activity or expression comprises an antibody, a small molecule, a direct inhibitor of CARD9, an indirect inhibitor of CARD9, an allosteric modulator of CARD9, an anti-CARD9 antibody or antibody fragment, antibody or antibody fragment that specifically binds to Rubicon, an anti-Tripartite Motif Containing 62 (TRIM62) antibody or antibody fragment, an antibody or antibody fragment that specifically binds to B Cell CLL/Lymphoma 10 (BCL10), an inhibitor of CARD9-Rubicon interaction, an inhibitor of CARD9-Tripartite Motif Containing 62 (TRIM62) interaction, an inhibitor of CARD9-B Cell CLL/Lymphoma 10 (BCL10) interaction, a small molecule that specifically binds CARD9, a small molecule that specifically binds to Rubicon, a small molecule that specifically binds to Tripartite Motif Containing 62 (TRIM62), a small molecule that specifically binds to B Cell CLL/Lymphoma 10 (BCL10), an inhibitor of CARD9-Rubicon interaction, an inhibitor of CARD9-Tripartite Motif Containing 62 (TRIM62) interaction, an inhibitor of B Cell CLL/Lymphoma 10 (BCL10)-CARD9 interaction or a combination thereof.
78. The method of paragraph 77, wherein the inhibitor of CARD9 activity or expression comprises an indirect inhibitor of CARD9.
79. A method of treating a disease or condition in a subject comprising administering to the subject a therapeutically effective amount of an inhibitor of caspase recruitment domain family member 9 (CARD9) activity or expression, provided one or more serological markers is detected in a biological sample obtained from the subject.
80. The method of paragraph 79, wherein the one or more serological markers is detected using an enzyme-linked immunosorbent assay (ELISA), a single molecule array (Simoa), immunohistochemistry, internal transcribed spacer (ITS) sequencing, or any combination thereof.
81. A method of treating a disease or condition in a subject comprising administering to the subject a therapeutically effective amount of an inhibitor of caspase recruitment domain family member 9 (CARD9) activity or expression, provided an elevated level of one or more serological markers is detected in a biological sample obtained from the subject, as compared to a level of the one or more serological markers in an individual who does not have the disease or condition.
82. The method of paragraph 81, wherein the elevated level of the one or more serological markers is detected using an enzyme-linked immunosorbent assay (ELISA), a single molecule array (Simoa), immunohistochemistry, internal transcribed spacer (ITS) sequencing, or any combination thereof.
83. The method of paragraphs 79 or 81, wherein one or more genetic risk variants and/or biomarkers is detected in the biological sample obtained from the subject.
84. The method of paragraph 83, wherein the one or more genetic risk variants comprise single nucleotide variants within the caspase recruitment domain family member 9 (CARD9) gene locus.
85. The method of paragraph 83, wherein the one or more genetic risk variants comprise single nucleotide variants within the c-type lectin domain containing 7A (CLEC7A) gene locus.
86. The method of paragraph 83, wherein the one or more genetic risk variants comprises one copy (heterozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
87. The method of paragraph 83, wherein the one or more genetic risk variants comprises two copies (homozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
88. The method of paragraph 83, wherein the risk allele at rs4077515 comprises an “A”, the risk allele at rs10870077 comprises a “C”, the risk allele at rs2078178 comprises a “G” or an “A”, and the risk allele at rs16910631 comprises a “G”.
89. The method of paragraph 83, wherein the one or more genetic risk variants and/or biomarkers is associated with a risk that the subject has, or will develop, intestinal mycobiota characterized by an increase in Malassezia spp. or Cladosporium spp.
90. The method of paragraph 83, wherein the one or more genetic risk variants and/or biomarkers is detected using polymerase chain reaction (PCR), reverse-transcription polymerase chain reaction (RT-PCR), a genotyping array, automated sequencing, or a combination thereof.
91. A method of inhibiting or reducing caspase recruitment domain family member 9 (CARD9) activity or expression in a subject suffering from a disease or condition, comprising:
   subjecting a biological sample obtained from a subject suffering from a disease or condition to an assay suitable to detect one or more serological markers; and
   administering to the subject a therapeutically effective amount of an inhibitor of caspase recruitment domain family member 9 (CARD9), provided the one or more serological markers is detected in the biological sample obtained from the subject.
92. The method of paragraph 91, wherein the assay suitable to detect the one or more serological markers comprises enzyme-linked immunosorbent assay (ELISA), a single molecule array (Simoa), immunohistochemistry, internal transcribed spacer (ITS) sequencing, or any combination thereof.
93. A method of inhibiting or reducing caspase recruitment domain family member 9 (CARD9) activity or expression in a subject suffering from a disease or condition, comprising:
   subjecting a biological sample obtained from a subject suffering from a disease or condition to an assay suitable to detect a level of one or more serological markers; and
   administering to the subject a therapeutically effective amount of an inhibitor of caspase recruitment domain family member 9 (CARD9), provided an elevated level of the one or more serological markers is detected in the biological sample obtained from the subject, as compared to a level of the one or more serological markers in an individual who does not have the disease or condition.
94. The method of paragraph 93, wherein the assay suitable to detect the level of the one or more serological markers comprises enzyme-linked immunosorbent assay (ELISA), a single molecule array (Simoa), immunohistochemistry, internal transcribed spacer (ITS) sequencing, or any combination thereof.
95. The method of paragraphs 91 or 93, further comprising:
   subjecting a biological sample obtained from a subject suffering from a disease or condition to an assay suitable to detect one or more genetic risk variants and/or biomarkers; and
   administering to the subject a therapeutically effective amount of an inhibitor of CARD9, provided the one or more genetic risk variants and/or biomarkers is detected in the biological sample obtained from the subject.
96. The method of paragraph 95, wherein the one or more genetic risk variants comprise single nucleotide variants within the caspase recruitment domain family member 9 (CARD9) gene locus.
97. The method of paragraph 95, wherein the one or more genetic risk variants comprise single nucleotide variants within the c-type lectin domain containing 7A (CLEC7A) gene locus.
98. The method of paragraph 95, wherein the one or more genetic risk variants comprises one copy (heterozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
99. The method of paragraph 95, wherein the one or more genetic risk variants comprises two copies (homozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
100. The method of paragraph 95, wherein the risk allele at rs4077515 comprises an “A”, the risk allele at rs10870077 comprises a “C”, the risk allele at rs2078178 comprises a “G” or an “A”, and the risk allele at rs16910631 comprises a “G”.
101. The method of paragraph 95, wherein the one or more genetic risk variants and/or biomarkers is associated with a risk that the subject has, or will develop, intestinal mycobiota characterized by an increase in Malassezia spp. or Cladosporium spp.
102. The method of paragraph 95, wherein the assay suitable to detect one or more genetic risk variants and/or biomarkers comprises polymerase chain reaction (PCR), reverse-transcription polymerase chain reaction (RT-PCR), a genotyping array, automated sequencing, or a combination thereof.
103. The method of any of paragraphs 79, 81, 91 or 93, wherein the one or more serological markers comprises anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globosa antibody, anti-Cladosporium spp. antibody, or a combination thereof.
104. The method of any of paragraphs 79, 81, 91 or 93, wherein the disease or condition comprises inflammatory bowel disease, Crohn's disease, or ulcerative colitis.
105. The method of paragraph 103, wherein the ulcerative colitis comprises medically refractory ulcerative colitis.
106. The method of paragraph 103, wherein the Crohn's disease comprises medically refractory Crohn's disease.
107. The method of any of paragraphs 79, 81, 91 or 93, wherein the subject is a mammal.
108. The method of any of paragraphs 79, 81, 91 or 93, wherein the subject is a human.
109. The method of any of paragraphs 79, 81, 91 or 93, wherein the biological sample comprises tissue biopsy, whole blood, plasma, serum, fecal specimen, urine, or saliva.
110. The method of any of paragraphs 79, 81, 91 or 93, wherein the inhibitor of CARD9 activity or expression comprises an antibody, a small molecule, a direct inhibitor of CARD9, an indirect inhibitor of CARD9, an allosteric modulator of CARD9, an anti-CARD9 antibody or antibody fragment, antibody or antibody fragment that specifically binds to Rubicon, an anti-Tripartite Motif Containing 62 (TRIM62) antibody or antibody fragment, an antibody or antibody fragment that specifically binds to B Cell CLL/Lymphoma 10 (BCL10), an inhibitor of CARD9-Rubicon interaction, an inhibitor of CARD9-Tripartite Motif Containing 62 (TRIM62) interaction, an inhibitor of CARD9-B Cell CLL/Lymphoma 10 (BCL10) interaction, a small molecule that specifically binds CARD9, a small molecule that specifically binds to Rubicon, a small molecule that specifically binds to Tripartite Motif Containing 62 (TRIM62), a small molecule that specifically binds to B Cell CLL/Lymphoma 10 (BCL10), an inhibitor of CARD9-Rubicon interaction, an inhibitor of CARD9-Tripartite Motif Containing 62 (TRIM62) interaction, an inhibitor of B Cell CLL/Lymphoma 10 (BCL10)-CARD9 interaction or a combination thereof.
111. The method of any of paragraphs 79, 81, 91 or 93, wherein the inhibitor of CARD9 activity or expression comprises an indirect inhibitor of CARD9.
112. A method of treating a disease or condition in a subject comprising administering to the subject a therapeutically effective amount of a modulator of dendritic cell-associated c-type lectin-1 (Dectin-1A) activity or expression, provided one or more genetic risk variants and/or biomarkers is detected in a biological sample obtained from the subject.
113. The method of paragraph 112, wherein the one or more genetic risk variants and/or biomarkers is detected using polymerase chain reaction (PCR), reverse-transcription polymerase chain reaction (RT-PCR), a genotyping array, automated sequencing, or a combination thereof.
114. The method of paragraph 112, provided a presence or a level of one or more serological markers is detected in the biological sample obtained from the subject.
115. The method of paragraph 114, wherein the one or more serological markers comprises anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globosa antibody, anti-Cladosporium spp. antibody, or a combination thereof.
116. The method of paragraph 114, wherein the presence or the level of the one or more serological markers is detected using an enzyme-linked immunosorbent assay (ELISA), a single molecule array (Simoa), immunohistochemistry, internal transcribed spacer (ITS) sequencing, or any combination thereof.
117. A method of inhibiting or reducing dendritic cell-associated c-type lectin-1 (Dectin-1A) activity or expression in a subject suffering from a disease or condition, comprising:
   subjecting a biological sample obtained from a subject suffering from a disease or condition to an assay suitable to detect one or more genetic risk variants and/or biomarkers; and
   administering to the subject a therapeutically effective amount of a modulator of dendritic cell-associated c-type lectin-1 (Dectin-1A), provided the one or more genetic risk variants and/or biomarkers is detected in the biological sample obtained from the subject.
118. A method of activating or increasing dendritic cell-associated c-type lectin-1 (Dectin-1A) activity or expression in a subject suffering from a disease or condition, comprising:
   subjecting a biological sample obtained from a subject suffering from a disease or condition to an assay suitable to detect one or more genetic risk variants and/or biomarkers; and
   administering to the subject a therapeutically effective amount of a modulator of dendritic cell-associated c-type lectin-1 (Dectin-1A), provided the one or more genetic risk variants and/or biomarkers is detected in the biological sample obtained from the subject.
119. The method of paragraphs 117 or 118, wherein the assay suitable to detect the presence or the level of the one or more genetic risk variants and/or biomarkers comprises polymerase chain reaction (PCR), reverse-transcription polymerase chain reaction (RT-PCR), a genotyping array, automated sequencing, or a combination thereof.
120. The method of paragraphs 117 or 118, further comprising:
   subjecting a biological sample obtained from a subject suffering from a disease or condition to an assay suitable to detect a presence or a level of one or more serological markers; and
   administering to the subject the therapeutically effective amount of the modulator of Dectin-1A, provided the presence or the level of the one or more serological markers is detected in the biological sample obtained from the subject.
121. The method of paragraphs 117 or 118, wherein the one or more serological markers comprises anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globosa antibody, anti-Cladosporium spp. antibody, or a combination thereof.
122. The method of paragraphs 117 or 118, wherein the assay suitable to detect the presence or the level of the one or more serological markers comprises enzyme-linked immunosorbent assay (ELISA), a single molecule array (Simoa), immunohistochemistry, internal transcribed spacer (ITS) sequencing, or any combination thereof.
123. The method of any paragraphs 112, 117 or 118, wherein the one or more genetic risk variants comprise single nucleotide variants within the caspase recruitment domain family member 9 (CARD9) gene locus.
124. The method of any of paragraphs 112, 117 or 118, wherein the one or more genetic risk variants comprise single nucleotide variants within the c-type lectin domain containing 7A (CLEC7A) gene locus.
125. The method of any of paragraphs 112, 117 or 118, wherein the one or more genetic risk variants comprises one copy (heterozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
126. The method of any of paragraphs 112, 117 or 118, wherein the one or more genetic risk variants comprises two copies (homozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
127. The method of any of paragraphs 112, 117 or 118, wherein the risk allele at rs4077515 comprises an “A”, the risk allele at rs10870077 comprises a “C”, the risk allele at rs2078178 comprises a “G” or an “A”, and the risk allele at rs16910631 comprises a “G”.
128. The method of any of paragraphs 112, 117 or 118, wherein the one or more genetic risk variants and/or biomarkers is associated with a risk that the subject has, or will develop, intestinal mycobiota characterized by an increase in Malassezia spp. or Cladosporium spp.
129. The method of any of paragraphs 112, 117 or 118, wherein the disease or condition comprises inflammatory bowel disease, Crohn's disease, or ulcerative colitis.
130. The method of paragraph 129, wherein the ulcerative colitis comprises medically refractory ulcerative colitis.
131. The method of paragraph 129, wherein the Crohn's disease comprises medically refractory Crohn's disease.
132. The method of any of paragraphs 112, 117 or 118, wherein the subject is a mammal.
133. The method of any of paragraphs 112, 117 or 118, wherein the subject is a human.
134. The method of any of paragraphs 112, 117 or 118, wherein the biological sample comprises tissue biopsy, whole blood, plasma, serum, fecal specimen, urine, or saliva.
135. The method of any of paragraphs 112, 117 or 118, wherein the modulator of dendritic cell-associated c-type lectin-1 (Dectin-1A) activity or expression comprises an antibody or antibody fragment.
136. The method of any of paragraphs 112, 117 or 118, wherein the modulator of dendritic cell-associated c-type lectin-1 (Dectin-1A) activity or expression comprises an antibody or antibody fragment, a small molecule, an allosteric modulator, an agonist, an antagonist, a direct modulator of Dectin-1A, an indirect modulator of Dectin-1A.
137. A method of treating a disease or condition in a subject comprising administering to the subject a therapeutically effective amount of a modulator of dendritic cell-associated c-type lectin-1 (Dectin-1A) activity or expression, provided one or more serological markers is detected in a biological sample obtained from the subject.
138. The method of paragraph 137, wherein the one or more serological markers is detected using an enzyme-linked immunosorbent assay (ELISA), a single molecule array (Simoa), immunohistochemistry, internal transcribed spacer (ITS) sequencing, or any combination thereof.
139. A method of treating a disease or condition in a subject comprising administering to the subject a therapeutically effective amount of a modulator of dendritic cell-associated c-type lectin-1 (Dectin-1A) activity or expression, provided an elevated level of one or more serological markers is detected in a biological sample obtained from the subject, as compared to a level of the one or more serological markers in an individual who does not have the disease or condition.
140. The method of paragraph 139, wherein the elevated level of the one or more serological markers is detected using an enzyme-linked immunosorbent assay (ELISA), a single molecule array (Simoa), immunohistochemistry, internal transcribed spacer (ITS) sequencing, or any combination thereof.
141. The method of paragraphs 137 or 139, wherein one or more genetic risk variants and/or biomarkers is detected in the biological sample obtained from the subject.
142. The method of paragraph 141, wherein the one or more genetic risk variants comprise single nucleotide variants within the caspase recruitment domain family member 9 (CARD9) gene locus.
143. The method of paragraph 141, wherein the one or more genetic risk variants comprise single nucleotide variants within the c-type lectin domain containing 7A (CLEC7A) gene locus.
144. The method of paragraph 141, wherein the one or more genetic risk variants comprises one copy (heterozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
145. The method of paragraph 141, wherein the one or more genetic risk variants comprises two copies (homozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
146. The method of paragraph 141, wherein the risk allele at rs4077515 comprises an “A”, the risk allele at rs10870077 comprises a “C”, the risk allele at rs2078178 comprises a “G” or an “A”, and the risk allele at rs16910631 comprises a “G”.
147. The method of paragraph 141, wherein the one or more genetic risk variants and/or biomarkers is associated with a risk that the subject has, or will develop, intestinal mycobiota characterized by an increase in Malassezia spp. or Cladosporium spp.
148. The method of paragraph 141, wherein the one or more genetic risk variants and/or biomarkers is detected using polymerase chain reaction (PCR), reverse-transcription polymerase chain reaction (RT-PCR), a genotyping array, automated sequencing, or a combination thereof.
149. A method of inhibiting or reducing dendritic cell-associated c-type lectin-1 (Dectin-1A) activity or expression in a subject suffering from a disease or condition, comprising:
   subjecting a biological sample obtained from a subject suffering from a disease or condition to an assay suitable to detect one or more serological markers; and
   administering to the subject a therapeutically effective amount of a modulator of dendritic cell-associated c-type lectin-1 (Dectin-1A), provided the one or more serological markers is detected in the biological sample obtained from the subject.
150. The method of paragraph 149, wherein the one or more serological markers is detected using an enzyme-linked immunosorbent assay (ELISA), a single molecule array (Simoa), immunohistochemistry, internal transcribed spacer (ITS) sequencing, or any combination thereof.
151. A method of inhibiting or reducing dendritic cell-associated c-type lectin-1 (Dectin-1A) activity or expression in a subject suffering from a disease or condition, comprising:
   subjecting a biological sample obtained from a subject suffering from a disease or condition to an assay suitable to detect a level of one or more serological markers; and
   administering to the subject a therapeutically effective amount of a modulator of dendritic cell-associated c-type lectin-1 (Dectin-1A), provided an elevated level of the one or more serological markers is detected in the biological sample obtained from the subject, as compared the a level of the one or more serological markers in an individual who does not have the disease or condition.
152. The method of paragraph 151, wherein the assay suitable to detect the level of the one or more serological markers comprises enzyme-linked immunosorbent assay (ELISA), a single molecule array (Simoa), immunohistochemistry, internal transcribed spacer (ITS) sequencing, or any combination thereof.
153. The method of paragraphs 149 or 151, further comprising subjecting a biological sample obtained from a subject suffering from a disease or condition to an assay suitable to detect one or more genetic risk variants and/or biomarkers; and administering to the subject a therapeutically effective amount of a modulator of Dectin-1A, provided the one or more genetic risk variants and/or biomarkers is detected in the biological sample obtained from the subject.
154. The method of paragraph 153, wherein the assay suitable to detect one or more genetic risk variants and/or biomarkers comprises polymerase chain reaction (PCR), reverse-transcription polymerase chain reaction (RT-PCR), a genotyping array, automated sequencing, or a combination thereof.
155. The method of any of paragraphs 137, 139, 149 or 151, wherein the one or more serological markers comprises anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globosa antibody, anti-Cladosporium spp. antibody, or a combination thereof.
156. The method of paragraphs 137, 139, 149 or 151, wherein the disease or condition comprises inflammatory bowel disease, Crohn's disease, or ulcerative colitis.
157. The method of paragraph 156, wherein the ulcerative colitis comprises medically refractory ulcerative colitis.
158. The method of paragraph 156, wherein the Crohn's disease comprises medically refractory Crohn's disease.
159. The method of paragraph 156, wherein the subject is a mammal.
160. The method of paragraph 156, wherein the subject is a human.
161. The method of any of paragraphs 137, 139, 149 or 151, wherein the biological sample comprises tissue biopsy, whole blood, plasma, serum, fecal specimen, urine, or saliva.
162. The method of any of paragraphs 137, 139, 149 or 151, wherein the modulator of dendritic cell-associated c-type lectin-1 (Dectin-1A) activity or expression comprises an antibody or antibody fragment, a small molecule, an allosteric modulator, an agonist, an antagonist, a direct modulator of Dectin-1A, an indirect modulator of Dectin-1A.
163. A method of treating a disease or condition in a subject comprising administering to the subject a therapeutically effective amount of an antimycotic agent, provided one or more genetic risk variants and/or biomarkers is detected in a biological sample obtained from the subject.
164. The method of paragraph 163, wherein the one or more genetic risk variants and/or biomarkers is detected using polymerase chain reaction (PCR), reverse-transcription polymerase chain reaction (RT-PCR), a genotyping array, automated sequencing, or a combination thereof provided a presence or a level of one or more serological markers is detected in the biological sample obtained from the subject.
165. The method of paragraph 164, wherein the one or more serological markers comprises anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globosa antibody, anti-Cladosporium spp. antibody, or a combination thereof.
166. The method of paragraph 165, wherein an assay suitable to detect the presence or the level of the one or more serological markers comprises enzyme-linked immunosorbent assay (ELISA), a single molecule array (Simoa), immunohistochemistry, internal transcribed spacer (ITS) sequencing, or any combination thereof.
167. A method of inhibiting or reducing fungal growth in a subject suffering from a disease or condition, comprising:
   subjecting a biological sample obtained from a subject suffering from a disease or condition to an assay suitable to detect one or more genetic risk variants and/or biomarkers; and
   administering to the subject a therapeutically effective amount of an antimycotic agent, provided the one or more genetic risk variants and/or biomarkers is detected in the biological sample obtained from the subject.
168. The method of paragraph 167, wherein the assay suitable to detect one or more genetic risk variants and/or biomarkers comprises polymerase chain reaction (PCR), reverse-transcription polymerase chain reaction (RT-PCR), a genotyping array, automated sequencing, or a combination thereof.
169. The method of paragraph 168, further comprising:
   subjecting a biological sample obtained from a subject suffering from a disease or condition to an assay suitable to detect a presence or a level of one or more serological markers; and
   administering to the subject the therapeutically effective amount of the antimycotic agent, provided the presence or the level of the one or more serological markers is detected in the biological sample obtained from the subject.
170. The method of paragraph 169, wherein the one or more serological markers comprises anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globosa antibody, anti-Cladosporium spp. antibody, or a combination thereof.
171. The method of paragraph 169, wherein the assay suitable to detect the presence or the level of the one or more serological markers comprises enzyme-linked immunosorbent assay (ELISA), a single molecule array (Simoa), immunohistochemistry, internal transcribed spacer (ITS) sequencing, or any combination thereof.
172. The method of paragraphs 163 or 167, wherein the one or more genetic risk variants comprise single nucleotide variants within the caspase recruitment domain family member 9 (CARD9) gene locus.
173. The method of paragraphs 163 or 167, wherein the one or more genetic risk variants comprise single nucleotide variants within the c-type lectin domain containing 7A (CLEC7A) gene locus.
174. The method of paragraphs 163 or 167, wherein the one or more genetic risk variants comprises one copy (heterozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
175. The method of paragraphs 163 or 167, wherein the one or more genetic risk variants comprises two copies (homozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
176. The method of paragraphs 163 or 167, wherein the risk allele at rs4077515 comprises an “A”.
177. The method of paragraphs 163 or 167, wherein the risk allele at rs10870077 comprises a “C”.
178. The method of paragraphs 163 or 167, wherein the risk allele at rs2078178 comprises a “G” or an “A”.
179. The method of paragraphs 163 or 167, wherein the risk allele at rs16910631 comprises a “G”.
180. The method of paragraphs 163 or 167, wherein the one or more genetic risk variants and/or biomarkers is associated with a risk that the subject has, or will develop, intestinal mycobiota characterized by an increase in Malassezia spp. or Cladosporium spp.
181. The method of paragraphs 163 or 167, wherein the disease or condition comprises inflammatory bowel disease, Crohn's disease, or ulcerative colitis.
182. The method of paragraph 181, wherein the ulcerative colitis comprises medically refractory ulcerative colitis.
183. The method of paragraph 182, wherein the Crohn's disease comprises medically refractory Crohn's disease.
184. The method of paragraphs 163 or 167, wherein the subject is a mammal.
185. The method of paragraphs 163 or 167, wherein the subject is a human.
186. The method of paragraphs 163 or 167, wherein the biological sample comprises tissue biopsy, whole blood, plasma, serum, fecal specimen, urine, or saliva.
187. The method of paragraphs 163 or 167, wherein the antimycotic agent comprises a fungicide agent or a fungistatic agent.
188. The method of paragraphs 163 or 167, wherein the antimycotic agent comprises a polyene, an azole, an echinocandin, an flucytosine, an allylamine, a tolnaftate, or griseofulvin.
189. The method of paragraph 188, wherein the azole comprises triazole, imidazole, clotrimazole, ketoconazole, itraconazole, terconazole, oxiconazole, miconazole, econazole, tioconazole, voriconazole, fluconazole, isavuconazole, itraconazole, pramiconazole, ravuconazole, or posaconazole.
190. The method of paragraph 188, wherein the polyene comprises amphotericin B, nystatin, or natamycin.
191. The method of paragraph 188, wherein the echinocandin comprises caspofungin, anidulafungin, or micafungin.
192. The method of paragraph 188, wherein the allylamine comprises naftifine or terbinafine.
193. A method of treating a disease or condition in a subject comprising administering to the subject a therapeutically effective amount of an antimycotic agent, provided one or more serological markers is detected in a biological sample obtained from the subject.
194. The method of paragraph 193, wherein the one or more serological markers is detected using an enzyme-linked immunosorbent assay (ELISA), a single molecule array (Simoa), immunohistochemistry, internal transcribed spacer (ITS) sequencing, or any combination thereof.
195. A method of treating a disease or condition in a subject comprising administering to the subject a therapeutically effective amount of an antimycotic agent, provided an elevated level of one or more serological markers is detected in a biological sample obtained from the subject.
196. The method of paragraph 195, wherein the elevated level of the one or more serological markers is detected using an enzyme-linked immunosorbent assay (ELISA), a single molecule array (Simoa), immunohistochemistry, internal transcribed spacer (ITS) sequencing, or any combination thereof.
197. The method of paragraphs 193 or 195, provided one or more genetic risk variants and/or biomarkers is detected in the biological sample obtained from the subject.
198. The method of paragraphs 193 or 195, wherein the one or more genetic risk variants comprise single nucleotide variants within the caspase recruitment domain family member 9 (CARD9) gene locus.
199. The method of paragraphs 193 or 195, wherein the one or more genetic risk variants comprise single nucleotide variants within the c-type lectin domain containing 7A (CLEC7A) gene locus.
200. The method of paragraphs 193 or 195, wherein the one or more genetic risk variants comprises one copy (heterozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
201. The method of paragraphs 193 or 195, wherein the one or more genetic risk variants comprises two copies (homozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
202. The method of paragraphs 200 or 201, wherein the risk allele at rs4077515 comprises an “A”.
203. The method of paragraphs 200 or 201, wherein the risk allele at rs10870077 comprises a “C”.
204. The method of paragraphs 200 or 201, wherein the risk allele at rs2078178 comprises a “G” or an “A”.
205. The method of paragraphs 200 or 201, wherein the risk allele at rs16910631 comprises a “G”.
206. The method of paragraphs 200 or 201, wherein the one or more genetic risk variants and/or serological markers is associated with a risk that the subject has, or will develop, intestinal mycobiota characterized by an increase in Malassezia spp. or Cladosporium spp.
207. The method of paragraphs 200 or 201, wherein the one or more genetic risk variants and/or serological markers is detected using polymerase chain reaction (PCR), reverse-transcription polymerase chain reaction (RT-PCR), a genotyping array, automated sequencing, or a combination thereof.
208. A method of inhibiting or reducing fungal growth in a subject suffering from a disease or condition, comprising:
   subjecting a biological sample obtained from a subject suffering from a disease or condition to an assay suitable to detect one or more serological markers; and
   administering to the subject a therapeutically effective amount of an antimycotic agent, provided the one or more serological markers is detected in the biological sample obtained from the subject.
209. The method of paragraph 208, wherein the assay suitable to detect the one or more serological markers comprises enzyme-linked immunosorbent assay (ELISA), a single molecule array (Simoa), immunohistochemistry, internal transcribed spacer (ITS) sequencing, or any combination thereof.
210. A method of inhibiting or reducing fungal growth in a subject suffering from a disease or condition, comprising:
   subjecting a biological sample obtained from a subject suffering from a disease or condition to an assay suitable to detect a level of one or more serological markers; and
   administering to the subject a therapeutically effective amount of an antimycotic agent, provided an elevated level of the one or more serological markers is detected in the biological sample obtained from the subject, as compared to a level of the one or more serological markers in an individual who does not have the disease or condition.
211. The method of paragraph 210, wherein the assay suitable to detect the level of the one or more serological markers comprises enzyme-linked immunosorbent assay (ELISA), a single molecule array (Simoa), immunohistochemistry, internal transcribed spacer (ITS) sequencing, or any combination thereof.
212. The method of paragraphs 208 or 210, further comprising:
   subjecting a biological sample obtained from a subject suffering from a disease or condition to an assay suitable to detect one or more genetic risk variants and/or biomarkers; and
   administering to the subject a therapeutically effective amount of an antimycotic agent, provided the one or more genetic risk variants and/or biomarkers is detected in the biological sample obtained from the subject.
213. The method of paragraphs 208 or 210, wherein the one or more genetic risk variants comprise single nucleotide variants within the caspase recruitment domain family member 9 (CARD9) gene locus.
214. The method of paragraphs 208 or 210, wherein the one or more genetic risk variants comprise single nucleotide variants within the c-type lectin domain containing 7A (CLEC7A) gene locus.
215. The method of paragraphs 208 or 210, wherein the one or more genetic risk variants comprises one copy (heterozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
216. The method of paragraphs 208 or 210, wherein the one or more genetic risk variants comprises two copies (homozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
217. The method of paragraphs 215 or 216, wherein the risk allele at rs4077515 comprises an “A”.
218. The method of paragraphs 215 or 216, wherein the risk allele at rs10870077 comprises a “C”.
219. The method of paragraphs 215 or 216, wherein the risk allele at rs2078178 comprises a “G” or an “A”.
220. The method of paragraphs 215 or 216, wherein the risk allele at rs16910631 comprises a “G”.
221. The method of paragraphs 208 or 210, wherein the one or more genetic risk variants and/or biomarkers is associated with a risk that the subject has, or will develop, intestinal mycobiota characterized by an increase in Malassezia spp. or Cladosporium spp.
222. The method of paragraphs 208 or 210, wherein the assay suitable to detect one or more genetic risk variants and/or biomarkers comprises polymerase chain reaction (PCR), reverse-transcription polymerase chain reaction (RT-PCR), a genotyping array, automated sequencing, or a combination thereof.
223. The method of any of paragraphs 193, 195, 208, or 210, wherein the one or more serological markers comprises anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globosa antibody, anti-Cladosporium spp. antibody, or a combination thereof.
224. The method of any of paragraphs 193, 195, 208, or 210, wherein the disease or condition comprises inflammatory bowel disease, Crohn's disease, or ulcerative colitis.
225. The method of any of paragraphs 193, 195, 208, or 210, wherein the ulcerative colitis comprises medically refractory ulcerative colitis.
226. The method of any of paragraphs 193, 195, 208, or 210, wherein the Crohn's disease comprises medically refractory Crohn's disease.
227. The method of any of paragraphs 193, 195, 208, or 210, wherein the subject is a mammal.
228. The method of any of paragraphs 193, 195, 208, or 210, wherein the subject is a human.
229. The method of any of paragraphs 193, 195, 208, or 210, wherein the biological sample comprises tissue biopsy, whole blood, plasma, serum, fecal specimen, urine, or saliva.
230. The method of any of paragraphs 193, 195, 208, or 210, wherein the antimycotic agent comprises a polyene, an azole, an echinocandin, an flucytosine, an allylamine, a tolnaftate, or griseofulvin.
231. The method of paragraph 230, wherein the azole comprises triazole, imidazole, clotrimazole, ketoconazole, itraconazole, terconazole, oxiconazole, miconazole, econazole, tioconazole, voriconazole, fluconazole, isavuconazole, itraconazole, pramiconazole, ravuconazole, or posaconazole.
232. The method of paragraph 230, wherein the polyene comprises amphotericin B, nystatin, or natamycin.
233. The method of paragraph 230, wherein the echinocandin comprises caspofungin, anidulafungin, or micafungin.
234. The method of paragraph 230, wherein the allylamine comprises naftifine or terbinafine.
235. A method of diagnosing a disease or condition in a subject, the method comprising:
   obtaining a biological sample from a subject;
   subjecting the biological sample to an assay suitable to detect one or more genetic risk variants and/or biomarkers; and diagnosing the subject with the disease or condition, provided the one or more genetic risk variants and/or biomarkers are detected in the biological sample obtained from the subject.
236. The method of paragraph 235, wherein the one or more genetic risk variants comprise single nucleotide variants within the caspase recruitment domain family member 9 (CARD9) gene locus.
237. The method of paragraph 235, wherein the one or more genetic risk variants comprise single nucleotide variants within the c-type lectin domain containing 7A (CLEC7A) gene locus.
238. The method of paragraph 235, wherein the one or more genetic risk variants comprises one copy (heterozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
239. The method of paragraph 235, wherein the one or more genetic risk variants comprises two copies (homozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
240. The method of paragraphs 238 or 239, wherein the risk allele at rs4077515 comprises an “A”.
241. The method of paragraphs 238 or 239, wherein the risk allele at rs10870077 comprises a “C”.
242. The method of paragraphs 238 or 239, wherein the risk allele at rs2078178 comprises a “G” or an “A”.
243. The method of paragraphs 238 or 239, wherein the risk allele at rs16910631 comprises a “G”.
244. The method of paragraph 235, wherein the one or more genetic risk variants and/or biomarkers is associated with a risk that the subject has, or will develop, intestinal mycobiota characterized by an increase in Malassezia spp. or Cladosporium spp.
245. The method of paragraph 235, wherein the disease or condition comprises inflammatory bowel disease, Crohn's disease, or ulcerative colitis.
246. The method of paragraph 245, wherein the ulcerative colitis comprises medically refractory ulcerative colitis.
247. The method of paragraph 245, wherein the Crohn's disease comprises medically refractory Crohn's disease.
248. The method of paragraph 235, wherein the subject is a mammal.
249. The method of paragraph 235, wherein the subject is a human.
250. The method of paragraph 235, wherein the biological sample comprises tissue biopsy, whole blood, plasma, serum, fecal specimen, urine, or saliva.
251. The method of paragraph 235, further comprising administering a therapeutically effective amount of an active agent, provided the subject is diagnosed with the disease or condition.
252. The method of paragraph 251, wherein active agent comprises an antimycotic agent, an inhibitor of caspase recruitment domain family member 9 (CARD9) activity or expression, or a modulator of dendritic cell-associated c-type lectin-1 (Dectin-1A) activity or expression.
253. The method of paragraph 252, wherein the antimycotic agent comprises a fungicide agent or a fungistatic agent.
254. The method of paragraph 252, wherein the antimycotic agent comprises a polyene, an azole, an echinocandin, an flucytosine, an allylamine, a tolnaftate, or griseofulvin.
255. The method of paragraph 254, wherein the azole comprises triazole, imidazole, clotrimazole, ketoconazole, itraconazole, terconazole, oxiconazole, miconazole, econazole, tioconazole, voriconazole, fluconazole, isavuconazole, itraconazole, pramiconazole, ravuconazole, or posaconazole.
256. The method of paragraph 254, wherein the polyene comprises amphotericin B, nystatin, or natamycin.
257. The method of paragraph 254, wherein the echinocandin comprises caspofungin, anidulafungin, or micafungin.
258. The method of paragraph 254, wherein the allylamine comprises naftifine or terbinafine.
259. The method of paragraph 254, wherein the inhibitor of CARD9 activity or expression comprises an antibody.
260. The method of paragraph 252, wherein the inhibitor of CARD9 activity or expression comprises a small molecule.
261. The method of paragraph 252, wherein the inhibitor of CARD9 activity or expression comprises a direct inhibitor of CARD9.
262. The method of paragraph 252, wherein the inhibitor of CARD9 activity or expression comprises an indirect inhibitor of CARD9.
263. The method of paragraph 252, wherein the inhibitor of CARD9 activity or expression comprises an allosteric modulator of CARD9.
264. The method of paragraph 252, wherein the inhibitor of CARD9 activity or expression comprises an anti-CARD9 antibody or antibody fragment.
265. The method of paragraph 252, wherein the inhibitor of CARD9 activity or expression comprises an antibody or antibody fragment that specifically binds to Rubicon.
266. The method of paragraph 252, wherein the inhibitor of CARD9 activity or expression comprises an anti-Tripartite Motif Containing 62 (TRIM62) antibody or antibody fragment.
267. The method of paragraph 252, wherein the inhibitor of CARD9 activity or expression comprises an antibody or antibody fragment that specifically binds to B Cell CLL/Lymphoma 10 (BCL10).
268. The method of paragraph 252, wherein the inhibitor of CARD9 activity or expression comprises an inhibitor of CARD9-Rubicon interaction.
269. The method of paragraph 252, wherein the inhibitor of CARD9 activity or expression comprises an inhibitor of CARD9-Tripartite Motif Containing 62 (TRIM62) interaction.
270. The method of paragraph 252, wherein the inhibitor of CARD9 activity or expression comprises an inhibitor of CARD9-B Cell CLL/Lymphoma 10 (BCL10) interaction.
271. The method of paragraph 252, wherein the inhibitor of CARD9 activity or expression comprises a small molecule that specifically binds CARD9.
272. The method of paragraph 252, wherein the inhibitor of CARD9 activity or expression comprises a small molecule that specifically binds to Rubicon.
273. The method of paragraph 252, wherein the inhibitor of CARD9 activity or expression comprises a small molecule that specifically binds to Tripartite Motif Containing 62 (TRIM62).
274. The method of paragraph 252, wherein the inhibitor of CARD9 activity or expression comprises a small molecule that specifically binds to B Cell CLL/Lymphoma 10 (BCL10).
275. The method of paragraph 252, wherein the inhibitor of CARD9 activity or expression comprises an inhibitor of CARD9-Rubicon interaction.
276. The method of paragraph 252, wherein the inhibitor of CARD9 activity or expression comprises an inhibitor of CARD9-Tripartite Motif Containing 62 (TRIM62) interaction.
277. The method of paragraph 252, wherein the inhibitor of CARD9 activity or expression comprises an inhibitor of B Cell CLL/Lymphoma 10 (BCL10)-CARD9 interaction.
278. The method of paragraph 252, wherein the modulator of dendritic cell-associated c-type lectin-1 (Dectin-1A) activity or expression comprises an antibody or antibody fragment.
279. The method of paragraph 252, wherein the modulator of dendritic cell-associated c-type lectin-1 (Dectin-1A) activity or expression comprises a small molecule.
280. The method of paragraph 252, wherein the modulator of dendritic cell-associated c-type lectin-1 (Dectin-1A) activity or expression comprises an allosteric modulator.
281. The method of paragraph 252, wherein the modulator of dendritic cell-associated c-type lectin-1 (Dectin-1A) activity or expression comprises an agonist.
282. The method of paragraph 252, wherein the modulator of dendritic cell-associated c-type lectin-1 (Dectin-1A) activity or expression comprises an antagonist.
283. The method of paragraph 252, wherein the modulator of dendritic cell-associated c-type lectin-1 (Dectin-1A) activity or expression comprises a direct modulator of Dectin-1A.
284. The method of paragraph 252, wherein the modulator of dendritic cell-associated c-type lectin-1 (Dectin-1A) activity or expression comprises an indirect modulator of Dectin-1A.
285. The method of paragraph 235, further comprising:
   subjecting a biological sample obtained from a subject suffering from a disease or condition to an assay suitable to detect a presence or a level of one or more serological markers; and
   diagnosing the subject with the disease or condition, provided the presence or the level of the one or more serological markers is detected in the biological sample obtained from the subject.
286. The method of 285, wherein the one or more serological markers comprises anti-Saccharomyces cerevisiae antibody (ASCA), an anti-neutrophil cytoplasmic antibody (ANCA), E. coli outer membrane porn protein C (OmpC), anti-Malassezia restricta antibody, anti-Malassezia pachydermatis antibody, anti-Malassezia furfur antibody, anti-Malassezia globosa antibody, anti-Cladosporium spp. antibody, or a combination thereof.
287. The method of 285, wherein the assay suitable to detect the presence of the level of the one or more serological markers comprises enzyme-linked immunosorbent assay (ELISA), a single molecule array (Simoa), immunohistochemistry, internal transcribed spacer (ITS) sequencing, or any combination thereof.
288. The method of 285, wherein the assay suitable to detect one or more genetic risk variants and/or biomarkers comprises polymerase chain reaction (PCR), reverse-transcription polymerase chain reaction (RT-PCR), a genotyping array, automated sequencing, or a combination thereof.
289. A method of diagnosing a disease or condition in a subject, the method comprising:
   obtaining a biological sample from a subject;
   subjecting the biological sample to an assay suitable to detect one or more serological markers; diagnosing the subject with the disease or condition, provided the one or more serological markers is detected in the biological sample obtained from the subject.
290. The method of paragraph 289, wherein the assay suitable to detect the one or more serological markers comprises enzyme-linked immunosorbent assay (ELISA), a single molecule array (Simoa), immunohistochemistry, internal transcribed spacer (ITS) sequencing, or any combination thereof.
291. A method of diagnosing a disease or condition in a subject, the method comprising:
   obtaining a biological sample from a subject;
   subjecting the biological sample to an assay suitable to detect a level of one or more serological markers;
   diagnosing the subject with the disease or condition, provided an elevated level of the one or more serological markers is detected in the biological sample obtained from the subject, as compared to a level of the one or more serological markers in an individual who does not have the disease or condition.
292. The method of paragraph 291, wherein the assay suitable to detect the level of the one or more serological markers comprises enzyme-linked immunosorbent assay (ELISA), a single molecule array (Simoa), immunohistochemistry, internal transcribed spacer (ITS) sequencing, or any combination thereof.
293. The method of paragraphs 289 or 291, wherein the one or more serological markers comprises anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globosa antibody, anti-Cladosporium spp. antibody, or a combination thereof.
294. The method of paragraphs 289 or 291, wherein the disease or condition comprises inflammatory bowel disease, Crohn's disease, or ulcerative colitis.
295. The method of paragraph 294, wherein the ulcerative colitis comprises medically refractory ulcerative colitis.
296. The method of paragraph 294, wherein the Crohn's disease comprises medically refractory Crohn's disease.
297. The method of paragraphs 289 or 291, wherein the subject is a mammal.
298. The method of paragraphs 289 or 291, wherein the subject is a human.
299. The method of paragraphs 289 or 291, wherein the biological sample comprises tissue biopsy, whole blood, plasma, serum, fecal specimen, urine, or saliva.
300. The method of paragraphs 289 or 291, further comprising administering a therapeutically effective amount of an active agent, provided the subject is diagnosed with the disease or condition.
301. The method of paragraph 300, wherein active agent comprises an antimycotic agent, an inhibitor of caspase recruitment domain family member 9 (CARD9) activity or expression, or a modulator of dendritic cell-associated c-type lectin-1 (Dectin-1A) activity or expression.
302. The method of paragraph 301, wherein the antimycotic agent comprises a fungicide agent or a fungistatic agent.
303. The method of paragraph 301, wherein the antimycotic agent comprises a polyene, an azole, an echinocandin, an flucytosine, an allylamine, a tolnaftate, or griseofulvin.
304. The method of paragraph 303, wherein the azole comprises triazole, imidazole, clotrimazole, ketoconazole, itraconazole, terconazole, oxiconazole, miconazole, econazole, tioconazole, voriconazole, fluconazole, isavuconazole, itraconazole, pramiconazole, ravuconazole, or posaconazole.
305. The method of paragraph 303, wherein the polyene comprises amphotericin B, nystatin, or natamycin.
306. The method of paragraph 303, wherein the echinocandin comprises caspofungin, anidulafungin, or micafungin.
307. The method of paragraph 303, wherein the allylamine comprises naftifine or terbinafine.
308. The method of paragraph 301, wherein the inhibitor of CARD9 activity or expression comprises an antibody.
309. The method of paragraph 301, wherein the inhibitor of CARD9 activity or expression comprises a small molecule.
310. The method of paragraph 301, wherein the inhibitor of CARD9 activity or expression comprises a direct inhibitor of CARD9.
311. The method of paragraph 301, wherein the inhibitor of CARD9 activity or expression comprises an indirect inhibitor of CARD9.
312. The method of paragraph 301, wherein the inhibitor of CARD9 activity or expression comprises an allosteric modulator of CARD9.
313. The method of paragraph 301, wherein the inhibitor of CARD9 activity or expression comprises an anti-CARD9 antibody or antibody fragment.
314. The method of paragraph 301, wherein the inhibitor of CARD9 activity or expression comprises an antibody or antibody fragment that specifically binds to Rubicon.
315. The method of paragraph 301, wherein the inhibitor of CARD9 activity or expression comprises an anti-Tripartite Motif Containing 62 (TRIM62) antibody or antibody fragment.
316. The method of paragraph 301, wherein the inhibitor of CARD9 activity or expression comprises an antibody or antibody fragment that specifically binds to B Cell CLL/Lymphoma 10 (BCL10).
317. The method of paragraph 301, wherein the inhibitor of CARD9 activity or expression comprises an inhibitor of CARD9-Rubicon interaction.
318. The method of paragraph 301, wherein the inhibitor of CARD9 activity or expression comprises an inhibitor of CARD9-Tripartite Motif Containing 62 (TRIM62) interaction.
319. The method of paragraph 301, wherein the inhibitor of CARD9 activity or expression comprises an inhibitor of CARD9-B Cell CLL/Lymphoma 10 (BCL10) interaction.
320. The method of paragraph 301, wherein the inhibitor of CARD9 activity or expression comprises a small molecule that specifically binds CARD9.
321. The method of paragraph 301, wherein the inhibitor of CARD9 activity or expression comprises a small molecule that specifically binds to Rubicon.
322. The method of paragraph 301, wherein the inhibitor of CARD9 activity or expression comprises a small molecule that specifically binds to Tripartite Motif Containing 62 (TRIM62).
323. The method of paragraph 301, wherein the inhibitor of CARD9 activity or expression comprises a small molecule that specifically binds to B Cell CLL/Lymphoma 10 (BCL10).
324. The method of paragraph 301, wherein the inhibitor of CARD9 activity or expression comprises an inhibitor of CARD9-Rubicon interaction.
325. The method of paragraph 301, wherein the inhibitor of CARD9 activity or expression comprises an inhibitor of CARD9-Tripartite Motif Containing 62 (TRIM62) interaction.
326. The method of paragraph 301, wherein the inhibitor of CARD9 activity or expression comprises an inhibitor of B Cell CLL/Lymphoma 10 (BCL10)-CARD9 interaction.
327. The method of paragraph 301, wherein the modulator of dendritic cell-associated c-type lectin-1 (Dectin-1A) activity or expression comprises an antibody or antibody fragment.
328. The method of paragraph 301, wherein the modulator of dendritic cell-associated c-type lectin-1 (Dectin-1A) activity or expression comprises a small molecule.
329. The method of paragraph 301, wherein the modulator of dendritic cell-associated c-type lectin-1 (Dectin-1A) activity or expression comprises an allosteric modulator.
330. The method of paragraph 301, wherein the modulator of dendritic cell-associated c-type lectin-1 (Dectin-1A) activity or expression comprises an agonist.
331. The method of paragraph 301, wherein the modulator of dendritic cell-associated c-type lectin-1 (Dectin-1A) activity or expression comprises an antagonist.
332. The method of paragraph 301, wherein the modulator of dendritic cell-associated c-type lectin-1 (Dectin-1A) activity or expression comprises a direct modulator of Dectin-1A.
333. The method of paragraph 301, wherein the modulator of dendritic cell-associated c-type lectin-1 (Dectin-1A) activity or expression comprises an indirect modulator of Dectin-1A.
334. The method of paragraphs 289 or 291, further comprising:
   subjecting the biological sample obtained from the subject suffering from the disease or condition to an assay suitable to detect one or more genetic risk variants and/or biomarkers; and
   diagnosing the subject with the disease or condition, provided the one or more genetic risk variants and/or biomarkers is detected in the biological sample obtained from the subject.
335. The method of paragraphs 289 or 291, wherein the one or more genetic risk variants comprise single nucleotide variants within the caspase recruitment domain family member 9 (CARD9) gene locus.
336. The method of paragraphs 289 or 291, wherein the one or more genetic risk variants comprise single nucleotide variants within the c-type lectin domain containing 7A (CLEC7A) gene locus.
337. The method of paragraphs 289 or 291, wherein the one or more genetic risk variants comprises one copy (heterozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
338. The method of paragraphs 289 or 291, wherein the one or more genetic risk variants comprises two copies (homozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
339. The method of paragraphs 337 or 338, wherein the risk allele at rs4077515 comprises an “A”.
340. The method of paragraphs 337 or 338, wherein the risk allele at rs10870077 comprises a “C”.
341. The method of paragraphs 337 or 338, wherein the risk allele at rs2078178 comprises a “G” or an “A”.
342. The method of paragraphs 337 or 338, wherein the risk allele at rs16910631 comprises a “G”.
343. The method of paragraphs 289 or 291, wherein the one or more genetic risk variants and/or biomarkers is associated with a risk that the subject has, or will develop, intestinal mycobiota characterized by an increase in Malassezia spp. or Cladosporium spp.
344. The method of paragraphs 289 or 291, wherein the assay suitable to detect one or more genetic risk variants and/or biomarkers comprises polymerase chain reaction (PCR), reverse-transcription polymerase chain reaction (RT-PCR), a genotyping array, automated sequencing, or a combination thereof.
345. A method of characterizing a subtype of a disease or condition, the method comprising: obtaining a biological sample from a subject;
   subjecting the biological sample to an assay suitable to detect one or more genetic risk variants and/or biomarkers; and
   characterizing the subtype of the disease or condition as medically refractory, provided the one or more genetic risk variants and/or biomarkers are detected in the biological sample obtained from the subject.
346. The method of paragraph 345, wherein the one or more genetic risk variants comprise single nucleotide variants within the caspase recruitment domain family member 9 (CARD9) gene locus.
347. The method of paragraph 345, wherein the one or more genetic risk variants comprise single nucleotide variants within the c-type lectin domain containing 7A (CLEC7A) gene locus.
348. The method of paragraph 345, wherein the one or more genetic risk variants comprises one copy (heterozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
349. The method of paragraph 345, wherein the one or more genetic risk variants comprises two copies (homozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
350. The method of paragraphs 348 or 349, wherein the risk allele at rs4077515 comprises an “A”.
351. The method of paragraphs 348 or 349, wherein the risk allele at rs10870077 comprises a “C”.
352. The method of paragraphs 348 or 349, wherein the risk allele at rs2078178 comprises a “G” or an “A”.
353. The method of paragraphs 348 or 349, wherein the risk allele at rs16910631 comprises a “G”.
354. The method of paragraph 345, wherein the one or more genetic risk variants and/or biomarkers is associated with a risk that the subject has, or will develop, intestinal mycobiota characterized by an increase in Malassezia spp. or Cladosporium spp.
355. The method of paragraph 354, wherein the disease or condition comprises inflammatory bowel disease, Crohn's disease, or ulcerative colitis.
356. The method of paragraph 345, wherein the subject is a mammal.
357. The method of paragraph 345, wherein the subject is a human.
358. The method of paragraph 345, wherein the biological sample comprises tissue biopsy, whole blood, plasma, serum, fecal specimen, urine, or saliva.
359. The method of paragraph 345, further comprising administering a therapeutically effective amount of an active agent, provided the subtype of the disease or condition is characterized as medically refractory.
360. The method of paragraph 359, wherein active agent comprises an antimycotic agent, an inhibitor of caspase recruitment domain family member 9 (CARD9) activity or expression, or a modulator of dendritic cell-associated c-type lectin-1 (Dectin-1A) activity or expression.
361. The method of paragraph 360, wherein the antimycotic agent comprises a fungicide agent or a fungistatic agent.
362. The method of paragraph 360, wherein the antimycotic agent comprises a polyene, an azole, an echinocandin, an flucytosine, an allylamine, a tolnaftate, or griseofulvin.
363. wherein the azole comprises triazole, imidazole, clotrimazole, ketoconazole, itraconazole, terconazole, oxiconazole, miconazole, econazole, tioconazole, voriconazole, fluconazole, isavuconazole, itraconazole, pramiconazole, ravuconazole, or posaconazole.
364. wherein the polyene comprises amphotericin B, nystatin, or natamycin.
365. wherein the echinocandin comprises caspofungin, anidulafungin, or micafungin.
366. wherein the allylamine comprises naftifine or terbinafine.
367. The method of paragraph 360, wherein the inhibitor of CARD9 activity or expression comprises an antibody.
368. The method of paragraph 360, wherein the inhibitor of CARD9 activity or expression comprises a small molecule.
369. The method of paragraph 360, wherein the inhibitor of CARD9 activity or expression comprises a direct inhibitor of CARD9.
370. The method of paragraph 360, wherein the inhibitor of CARD9 activity or expression comprises an indirect inhibitor of CARD9.
371. The method of paragraph 360, wherein the inhibitor of CARD9 activity or expression comprises an allosteric modulator of CARD9.
372. The method of paragraph 360, wherein the inhibitor of CARD9 activity or expression comprises an anti-CARD9 antibody or antibody fragment.
373. The method of paragraph 360, wherein the inhibitor of CARD9 activity or expression comprises an antibody or antibody fragment that specifically binds to Rubicon.
374. The method of paragraph 360, wherein the inhibitor of CARD9 activity or expression comprises an anti-Tripartite Motif Containing 62 (TRIM62) antibody or antibody fragment.
375. The method of paragraph 360, wherein the inhibitor of CARD9 activity or expression comprises an antibody or antibody fragment that specifically binds to B Cell CLL/Lymphoma 10 (BCL10).
376. The method of paragraph 360, wherein the inhibitor of CARD9 activity or expression comprises an inhibitor of CARD9-Rubicon interaction.
377. The method of paragraph 360, wherein the inhibitor of CARD9 activity or expression comprises an inhibitor of CARD9-Tripartite Motif Containing 62 (TRIM62) interaction.
378. The method of paragraph 360, wherein the inhibitor of CARD9 activity or expression comprises an inhibitor of CARD9-B Cell CLL/Lymphoma 10 (BCL10) interaction.
379. The method of paragraph 360, wherein the inhibitor of CARD9 activity or expression comprises a small molecule that specifically binds CARD9.
380. The method of paragraph 360, wherein the inhibitor of CARD9 activity or expression comprises a small molecule that specifically binds to Rubicon.
381. The method of paragraph 360, wherein the inhibitor of CARD9 activity or expression comprises a small molecule that specifically binds to Tripartite Motif Containing 62 (TRIM62).
382. The method of paragraph 360, wherein the inhibitor of CARD9 activity or expression comprises a small molecule that specifically binds to B Cell CLL/Lymphoma 10 (BCL10).
383. The method of paragraph 360, wherein the inhibitor of CARD9 activity or expression comprises an inhibitor of CARD9-Rubicon interaction.
384. The method of paragraph 360, wherein the inhibitor of CARD9 activity or expression comprises an inhibitor of CARD9-Tripartite Motif Containing 62 (TRIM62) interaction.
385. The method of paragraph 360, wherein the inhibitor of CARD9 activity or expression comprises an inhibitor of B Cell CLL/Lymphoma 10 (BCL10)-CARD9 interaction.
386. The method of paragraph 360, wherein the modulator of dendritic cell-associated c-type lectin-1 (Dectin-1A) activity or expression comprises an antibody or antibody fragment.
387. The method of paragraph 360, wherein the modulator of dendritic cell-associated c-type lectin-1 (Dectin-1A) activity or expression comprises a small molecule.
388. The method of paragraph 360, wherein the modulator of dendritic cell-associated c-type lectin-1 (Dectin-1A) activity or expression comprises an allosteric modulator.
389. The method of paragraph 360, wherein the modulator of dendritic cell-associated c-type lectin-1 (Dectin-1A) activity or expression comprises an agonist.
390. The method of paragraph 360, wherein the modulator of dendritic cell-associated c-type lectin-1 (Dectin-1A) activity or expression comprises an antagonist.
391. The method of paragraph 360, wherein the modulator of dendritic cell-associated c-type lectin-1 (Dectin-1A) activity or expression comprises a direct modulator of Dectin-1A.
392. The method of paragraph 360, wherein the modulator of dendritic cell-associated c-type lectin-1 (Dectin-1A) activity or expression comprises an indirect modulator of Dectin-1A.
393. The method of paragraph 345, further comprising:
   subjecting a biological sample obtained from a subject suffering from a disease or condition to an assay suitable to detect a presence or a level of one or more serological markers; and
   characterizing the subtype of the disease or condition as medically refractory, provided the presence or the level of the one or more serological markers is detected in the biological sample obtained from the subject.
394. The method of paragraph 393, wherein the one or more serological markers comprises anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Cladosporium spp. antibody, anti-Malassezia globosa antibody, or a combination thereof.
395. The method of paragraph 393, wherein the assay suitable to detect the presence or the level of the one or more serological markers comprises enzyme-linked immunosorbent assay (ELISA), a single molecule array (Simoa), immunohistochemistry, internal transcribed spacer (ITS) sequencing, or any combination thereof.
396. The method of paragraph 393, wherein the assay suitable to detect one or more genetic risk variants and/or biomarkers comprises polymerase chain reaction (PCR), reverse-transcription polymerase chain reaction (RT-PCR), a genotyping array, automated sequencing, or a combination thereof.
397. A method of characterizing a subtype of a disease or condition, the method comprising:
   obtaining a biological sample from a subject;
   subjecting the biological sample to an assay suitable to detect one or more serological markers; and
   characterizing the subtype of the disease or condition, provided the one or more serological markers is detected in the biological sample obtained from the subject.
398. The method of paragraph 397, wherein the assay suitable to detect the one or more serological markers comprises enzyme-linked immunosorbent assay (ELISA), a single molecule array (Simoa), immunohistochemistry, internal transcribed spacer (ITS) sequencing, or any combination thereof.
399. A method of characterizing a subtype of a disease or condition, the method comprising:
   obtaining a biological sample from a subject;
   subjecting the biological sample to an assay suitable to detect a level of one or more serological markers; and
   characterizing the subtype of the disease or condition, provided an elevated level of the one or more serological markers is detected in the biological sample obtained from the subject, as compared to a level of the one or more serological markers in an individual who does not have the disease or condition.
400. The method of paragraph 399, wherein the assay suitable to detect the level of the one or more serological markers comprises enzyme-linked immunosorbent assay (ELISA), a single molecule array (Simoa), immunohistochemistry, internal transcribed spacer (ITS) sequencing, or any combination thereof.
401. The method of paragraphs 397 or 399, wherein the one or more serological markers comprises anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Cladosporium spp. antibody, Malassezia globosa antibody or a combination thereof.
402. The method of paragraphs 397 or 399, wherein the disease or condition comprises inflammatory bowel disease, Crohn's disease, or ulcerative colitis.
403. The method of paragraphs 397 or 399, wherein the subtype comprises a medically refractor disease or condition.
404. The method of paragraph 402, wherein the Crohn's disease comprises medically refractory Crohn's disease.
405. The method of paragraphs 397 or 399, wherein the subject is a mammal.
406. The method of paragraphs 397 or 399, wherein the subject is a human.
407. The method of paragraphs 397 or 399, wherein the biological sample comprises tissue biopsy, whole blood, plasma, serum, fecal specimen, urine, or saliva.
408. The method of paragraphs 397 or 399, further comprising administering a therapeutically effective amount of an active agent, provided the subtype of the disease or condition is characterized as medically refractory.
409. The method of paragraph 408, wherein active agent comprises an antimycotic agent, an inhibitor of caspase recruitment domain family member 9 (CARD9) activity or expression, or a modulator of dendritic cell-associated c-type lectin-1 (Dectin-1A) activity or expression.
410. The method of paragraph 409, wherein the antimycotic agent comprises a fungicide agent or a fungistatic agent.
411. The method of paragraph 409, wherein the antimycotic agent comprises a polyene, an azole, an echinocandin, an flucytosine, an allylamine, a tolnaftate, or griseofulvin.
412. The method of paragraph 411, wherein the azole comprises triazole, imidazole, clotrimazole, ketoconazole, itraconazole, terconazole, oxiconazole, miconazole, econazole, tioconazole, voriconazole, fluconazole, isavuconazole, itraconazole, pramiconazole, ravuconazole, or posaconazole.
413. The method of paragraph 411, wherein the polyene comprises amphotericin B, nystatin, or natamycin.
414. The method of paragraph 411, wherein the echinocandin comprises caspofungin, anidulafungin, or micafungin.
415. The method of paragraph 411, wherein the allylamine comprises naftifine or terbinafine.
416. The method of paragraph 408, wherein the inhibitor of CARD9 activity or expression comprises an antibody.
417. The method of paragraph 408, wherein the inhibitor of CARD9 activity or expression comprises a small molecule.
418. The method of paragraph 408, wherein the inhibitor of CARD9 activity or expression comprises a direct inhibitor of CARD9.
419. The method of paragraph 408, wherein the inhibitor of CARD9 activity or expression comprises an indirect inhibitor of CARD9.
420. The method of paragraph 408, wherein the inhibitor of CARD9 activity or expression comprises an allosteric modulator of CARD9.
421. The method of paragraph 408, wherein the inhibitor of CARD9 activity or expression comprises an anti-CARD9 antibody or antibody fragment.
422. The method of paragraph 408, wherein the inhibitor of CARD9 activity or expression comprises an antibody or antibody fragment that specifically binds to Rubicon.
423. The method of paragraph 408, wherein the inhibitor of CARD9 activity or expression comprises an anti-Tripartite Motif Containing 62 (TRIM62) antibody or antibody fragment.
424. The method of paragraph 408, wherein the inhibitor of CARD9 activity or expression comprises an antibody or antibody fragment that specifically binds to B Cell CLL/Lymphoma 10 (BCL10).
425. The method of paragraph 408, wherein the inhibitor of CARD9 activity or expression comprises an inhibitor of CARD9-Rubicon interaction.
426. The method of paragraph 408, wherein the inhibitor of CARD9 activity or expression comprises an inhibitor of CARD9-Tripartite Motif Containing 62 (TRIM62) interaction.
427. The method of paragraph 408, wherein the inhibitor of CARD9 activity or expression comprises an inhibitor of CARD9-B Cell CLL/Lymphoma 10 (BCL10) interaction.
428. The method of paragraph 408, wherein the inhibitor of CARD9 activity or expression comprises a small molecule that specifically binds CARD9.
429. The method of paragraph 408, wherein the inhibitor of CARD9 activity or expression comprises a small molecule that specifically binds to Rubicon.
430. The method of paragraph 408, wherein the inhibitor of CARD9 activity or expression comprises a small molecule that specifically binds to Tripartite Motif Containing 62 (TRIM62).
431. The method of paragraph 408, wherein the inhibitor of CARD9 activity or expression comprises a small molecule that specifically binds to B Cell CLL/Lymphoma 10 (BCL10).
432. The method of paragraph 408, wherein the inhibitor of CARD9 activity or expression comprises an inhibitor of CARD9-Rubicon interaction.
433. The method of paragraph 408, wherein the inhibitor of CARD9 activity or expression comprises an inhibitor of CARD9-Tripartite Motif Containing 62 (TRIM62) interaction.
434. The method of paragraph 408, wherein the inhibitor of CARD9 activity or expression comprises an inhibitor of B Cell CLL/Lymphoma 10 (BCL10)-CARD9 interaction.
435. The method of paragraph 408, wherein the modulator of dendritic cell-associated c-type lectin-1 (Dectin-1A) activity or expression comprises an antibody or antibody fragment.
436. The method of paragraph 408, wherein the modulator of dendritic cell-associated c-type lectin-1 (Dectin-1A) activity or expression comprises a small molecule.
437. The method of paragraph 408, wherein the modulator of dendritic cell-associated c-type lectin-1 (Dectin-1A) activity or expression comprises an allosteric modulator.
438. The method of paragraph 408, wherein the modulator of dendritic cell-associated c-type lectin-1 (Dectin-1A) activity or expression comprises an agonist.
439. The method of paragraph 408, wherein the modulator of dendritic cell-associated c-type lectin-1 (Dectin-1A) activity or expression comprises an antagonist.
440. The method of paragraph 408, wherein the modulator of dendritic cell-associated c-type lectin-1 (Dectin-1A) activity or expression comprises a direct modulator of Dectin-1A.
441. The method of paragraph 408, wherein the modulator of dendritic cell-associated c-type lectin-1 (Dectin-1A) activity or expression comprises an indirect modulator of Dectin-1A.
442. The method of paragraphs 397 or 399, further comprising:
   subjecting the biological sample obtained from the subject suffering from the disease or condition to an assay suitable to detect one or more genetic risk variants and/or biomarkers; and
   characterizing the subtype of the disease or condition as medically refractor, provided the one or more genetic risk variants and/or biomarkers is detected in the biological sample obtained from the subject.
443. The method of paragraph 442, wherein the one or more genetic risk variants comprise single nucleotide variants within the caspase recruitment domain family member 9 (CARD9) gene locus.
444. The method of paragraph 442, wherein the one or more genetic risk variants comprise single nucleotide variants within the c-type lectin domain containing 7A (CLEC7A) gene locus.
445. The method of paragraph 442, wherein the one or more genetic risk variants comprises one copy (heterozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
446. The method of paragraph 442, wherein the one or more genetic risk variants comprises two copies (homozygous) of a risk allele at rs4077515, a risk allele at rs10870077, a risk allele at rs2078178, a risk allele at rs7959451, a risk allele at rs11053603, a risk allele at rs11053624, and/or a risk allele at rs16910631.
447. The method of paragraphs 445 or 446, wherein the risk allele at rs4077515 comprises an “A”.
448. The method of paragraphs 445 or 446, wherein the risk allele at rs10870077 comprises a “C”.
449. The method of paragraphs 445 or 446, wherein the risk allele at rs2078178 comprises a “G” or an “A”.
450. The method of paragraphs 445 or 446, wherein the risk allele at rs16910631 comprises a “G”.
451. The method of paragraphs 397 or 399, wherein the one or more genetic risk variants and/or biomarkers is associated with a risk that the subject has, or will develop, intestinal mycobiota characterized by an increase in Malassezia spp. or Cladosporium spp.
452. The method of paragraphs 397 or 399, wherein the assay suitable to detect one or more genetic risk variants and/or biomarkers comprises polymerase chain reaction (PCR), reverse-transcription polymerase chain reaction (RT-PCR), a genotyping array, automated sequencing, or a combination thereof.
453. A composition comprising at least 10 but less than 50 contiguous polynucleotide sequence of any one of SEQ ID NO: 1 (rs4077515), SEQ ID NO: 2 (rs10870077), SEQ ID NO: 3 (rs2078178), and SEQ ID NO: 4 (rs16910631), SEQ ID NO: 5 (rs7959451), SEQ ID NO: 6 rs11053603), and SEQ ID NO: 7 (rs11053624), or reverse complements thereof, wherein the contiguous polynucleotide sequence comprises a detectable molecule.
454. The composition of paragraph 453, wherein the detectable molecule comprises a fluorophore.
455. The composition of paragraph 453, wherein the polynucleotide sequences further comprises a quencher.
456. A method of detecting one or more genetic risk variants in a subject suffering from a disease or condition, the method comprising:
   contacting a biological sample comprising deoxyribonucleic acid (DNA) obtained from a subject with the composition of any of paragraphs 453-455 under conditions configured to hybridize the composition to the DNA from the subject; and
   detecting a presence or absence of a hybridization product comprising the DNA obtained from the subject and the composition by detecting the detectable molecule, wherein the presence of the hybridization product indicates a presence of one or more genetic risk variants in the subject.
457. A kit comprising the composition of any of paragraphs 453-455, and a primer pair configured to amplify a nucleic acid sequence comprising SEQ ID NOS: 8-14.
458. A composition comprising an antibody or antigen-binding fragment that specifically binds to CARD9, wherein the antibody or antigen-binding fragment comprises a detectable molecule.
459. The composition of paragraph 458, wherein the antibody comprises a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, or a combination thereof.
460. The composition of paragraph 458, wherein the antibody or antigen-binding fragment comprises an IgG antibody.
461. The composition of paragraph 458, wherein the antibody or antigen-binding fragment comprises an IgM antibody.
462. The composition of paragraph 458, wherein the antibody or antigen-binding fragment comprises an IgE antibody.
463. The composition of paragraph 458, wherein the detectable molecule comprises a fluorophore.
464. A method of detecting a level of CARD9 expression in a subject suffering from a disease or condition, the method comprising:
   contacting a biological sample obtained from a subject with the composition of any of paragraphs 458-463 under conditions configured to bind the composition to the CARD9; and
   detecting a presence or absence of the detectable molecule indicative of binding between the CARD9 and the composition.
465. A kit comprising the composition of any of paragraphs 458463, and a composition comprising an antibody or antigen-binding fragment that specifically binds to Malassezia spp. or Cladosporium spp., isolated polypeptides therefrom, or an anti-Malassezia spp. antibody or anti-Cladosporium spp. antibody, wherein the antibody or antigen-binding fragment comprises a paramagnetic particle.
466. A composition comprising an antibody or antigen-binding fragment that specifically binds to Malassezia spp. or Cladosporium spp., isolated polypeptides therefrom, or an anti-Malassezia spp. antibody or anti-Cladosporium spp. antibody, wherein the antibody or antigen-binding fragment comprises a detectable molecule.
467. The composition of paragraph 466, wherein the antibody comprises a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, or a combination thereof.
468. The composition of paragraph 466, wherein the antibody or antigen-binding fragment comprises an IgG antibody.
469. The composition of paragraph 466, wherein the antibody or antigen-binding fragment comprises an IgM antibody.
470. The composition of paragraph 466, wherein the antibody or antigen-binding fragment comprises an IgE antibody.
471. The composition of paragraph 466, wherein the detectable molecule comprises a fluorophore.
472. A method of detecting one or more serological markers in a subject suffering from a disease or condition, the method comprising:
   contacting a biological sample obtained from a subject with the composition of any of paragraphs 466-471 under conditions configured to bind the composition to Malassezia spp. or Cladosporium spp., isolated polypeptides therefrom, or an anti-Malassezia spp. antibody or anti-Cladosporium spp. antibody; and
   detecting a presence or absence of the detectable molecule indicative of binding between the Malassezia spp. or Cladosporium spp., the isolated polypeptides therefrom, or the anti-Malassezia spp. antibody or the anti-Cladosporium spp. antibody, or the isolated polypeptides therefrom, and the composition.
473. A kit comprising the composition of any of paragraphs 466-471, and a composition comprising an antibody or antigen-binding fragment that specifically binds to Malassezia spp. or Cladosporium spp., isolated polypeptides therefrom, or an anti-Malassezia spp. antibody or anti-Cladosporium spp. antibody, wherein the antibody or antigen-binding fragment comprises a paramagnetic particle.
474. A method of detecting one or more genetic risk variants in a subject suffering from a disease or condition, the method comprising:
   (a) contacting a biological sample comprising deoxyribonucleic acid (DNA) obtained from a subject suffering from a disease or condition with a composition sufficiently complementary to, and capable of, hybridizing to the one or more genetic risk variants, the composition comprising:
      (i) a labeled polynucleotide probe comprising SEQ ID NO: 1, or a reverse complement thereof,
      (ii) a labeled polynucleotide probe comprising SEQ ID NO: 2, or a reverse complement thereof,
      (iii) a labeled polynucleotide probe comprising SEQ ID NO: 3, or a reverse complement thereof,
      (iv) a labeled polynucleotide probe comprising SEQ ID NO: 4, or a reverse complement thereof,
      (v) a labeled polynucleotide probe comprising SEQ ID NO: 5, or a reverse complement thereof,
      (vi) a labeled polynucleotide probe comprising SEQ ID NO: 6, or a reverse complement thereof,
      (vii) a labeled polynucleotide probe comprising SEQ ID NO: 7, or a reverse complement thereof,
      (viii) a labeled polynucleotide probe comprising a nucleic acid sequence that differs from a probe selected from the group consisting of (i)-(vii) by up to three nucleobases, provided the detectably labeled polynucleotide probe hybridizes to the one or more genetic risk variants;
      (ix) a labeled polynucleotide probe comprising a nucleic acid sequence complementary to a probe selected from the group consisting of (i)-(viii); or
      (x) a combination of probes selected from the group consisting of (i)-(ix); and
   (b) detecting a presence or an absence of a hybridization product comprising the DNA obtained from the subject and the composition, by detecting the labeled polynucleotide probe.
475. The method of paragraph 474, wherein the labeled polynucleotide probe further comprises a fluorophore.
476. The method of paragraph 474, wherein the labeled polynucleotide probe further comprises a quencher.

### EXAMPLES

While preferred embodiments have been shown and described herein, it will be obvious to those skilled in the art that such embodiments are provided by way of example only. Numerous variations, changes, and substitutions will now occur to those skilled in the art without departing from the embodiments provided. It should be understood that various alternatives to the embodiments described herein may be employed.

### Example 1

Studies of inflammatory bowel disease (IBD) have focused on immune responses to gut bacteria, genetic and serological evidence suggests a role for altered immunity to fungal members of the intestinal microbiota in some patients. The inventors have characterized fungi associated with the intestinal mucosa in non-diseased people and in patients with Crohn's disease and have identified fungi that are specifically abundant in patients with Crohn's disease. One of these, a common skin resident fungus called Malassezia restricta, is also linked to the presence of an IBD-associated polymorphism in the gene for CARD9, a signaling adaptor protein important for host defense against fungi, and is recognized by patient anti-fungal antibodies. It was observed that M. restricta exacerbates colitis via CARD9 in mouse models of disease.

Inflammatory bowel disease (IBD) susceptibility and severity are understood to be influenced by a combination of genetics, microbiota, and environment. IBD-associated genetic risk factors have been linked to changes in the bacterial microbiota. The intestinal microbiota includes fungi (the mycobiota), and changes in the mycobiota have been reported in patients with Crohn's disease (CD), especially increased prevalence of Candida spp. CARD9 is a signaling adaptor protein that is essential for anti-fungal innate immunity in mice and humans, and polymorphisms in CARD9 have been linked to IBD. The inventors observed that mice lacking the gene for Dectin-1, an innate immune receptor that is essential for host defense against fungi, are more susceptible to experimental colitis, and that polymorphisms in this gene are associated with disease severity in IBD patients. Dectin-1, as well as related anti-fungal receptors, signal through an adaptor molecule called CARD9, and deletion of CARD9 renders mice and humans highly susceptible to fungal infection. Polymorphisms in the CARD9 gene are among the strongest genetic risk factors for Crohn's disease and ulcerative colitis. “Anti-Saccharomyces cerevisiae antibodies” (ASCA) that bind yeast cell wall mannans found in many, but not all, fungi have been recognized as a biomarker capable of identifying most patients with Crohn's disease (CD), suggesting that immune interactions with fungi commonly occur in the context of IBD. This suggests that immune responses to intestinal fungi may influence intestinal inflammation in a subset of patients with IBD.

Sequencing of highly variable regions in bacterial rDNA (16S sequencing) has proven to be a powerful and widely-adopted approach for in-depth characterization of complex bacterial communities. The analogous approach for fungi is based on sequencing the “Internal Transcribed Spacer (ITS)” regions of fungal rDNA. A recent study of fecal samples from pediatric IBD patients and controls using this approach suggested that disease may be associated with a decrease in overall fungal diversity and an increase in the presence of Candida spp. Subsequent studies with adult patients investigating fecal or biopsy samples have described similar associations with Candida. Using a fungal ITS sequencing approach and a custom-curated database of fungal ITS sequences, the inventors examined in depth the mucosa-associated intestinal mycobiota of non-diseased people and of patients with CD.

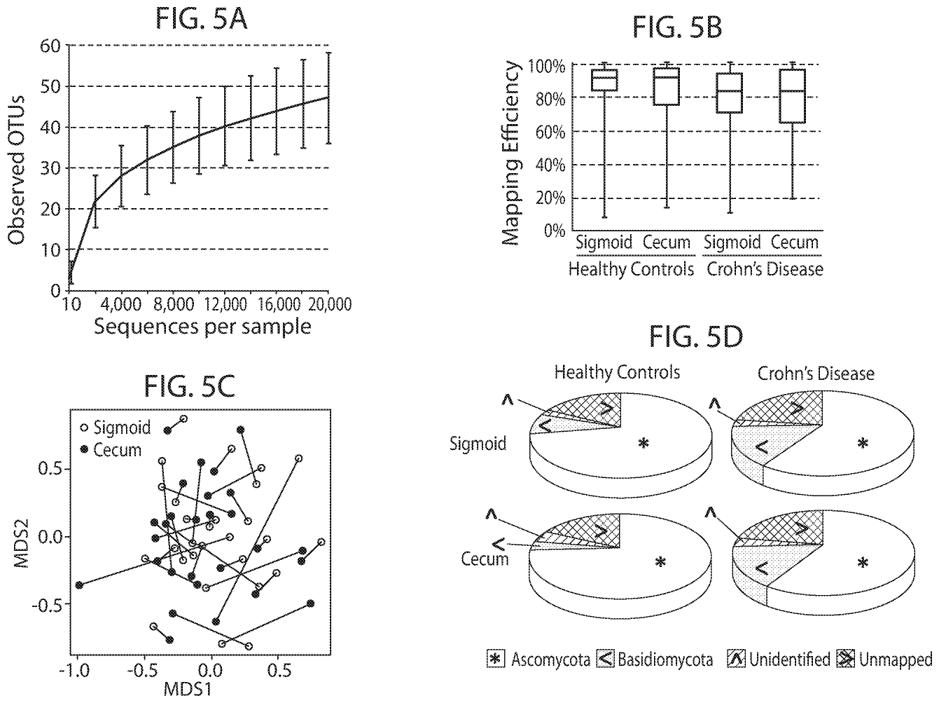

The inventors focused the mycobiota analysis on fungi that are associated with intestinal mucosal surfaces (as opposed to those found in fecal material), and obtained water-lavage samples from Crohn's disease patients and non-diseased controls undergoing screening colonic endoscopy (Table 1). The inventors isolated and sequenced fungal ITS1 regions from 166 samples representing the sigmoid colon and cecum (FIG. 5A). Three samples were excluded from further analysis due to obtaining too few sequences for sufficiently deep analysis. For the remaining samples, an average of ≈100,000 sequences per sample were assessed. Of these sequences, 77%-83% were identified by comparison to a custom-curated fungal ITS database, an approach designed to mitigate current challenges associated with inaccurate and non-specific data in public repositories (FIG. 5B). For 28 patients, the inventors paired sigmoid and cecum samples collected at the same time. Without being bound to any particular theory, analysis demonstrated that these samples are more closely related to each other than they were to the sites from which they were collected (FIG. 5C), suggesting that heterogeneity between samples is a reliable measure of the source of the samples and not due to technical variations in sample handling.

TABLE 1
Sequencing and cohort characteristics: Sample and ITS sequencing summary (upper table) and
Sample cohort summary (lower table)

| | Healthy Control | | Crohn's Disease | |
|---|---|---|---|---|
| | Sigmoid | Cecum | Sigmoid | Cecum |
| Total Samples | 55 | 55 | 34 | 22 |
| <10,000 Sequences Recovered | 1 | 1 | 1 | 0 |
| Usable Samples | 54 | 54 | 33 | 22 |
| Avg. # of Sequences/Sample | 103,140 | 96,035 | 148,969 | 129,111 |
| % Sequences Mapped | 82% | 81% | 76% | 76% |
| Average Age (± Std. Dev.) | 64 ± 12 | 64 ± 12 | 42 ± 1 | 44 ± 14 |
| Sex (M/F) | 40/14 | 36/18 | 14/19 | 11/11 |
| Caucasian/Asian/Black/Hispanic | 50/10/0/3 | 49/1/1/2 | 30/1/1/1 | 15/2/1/3 |

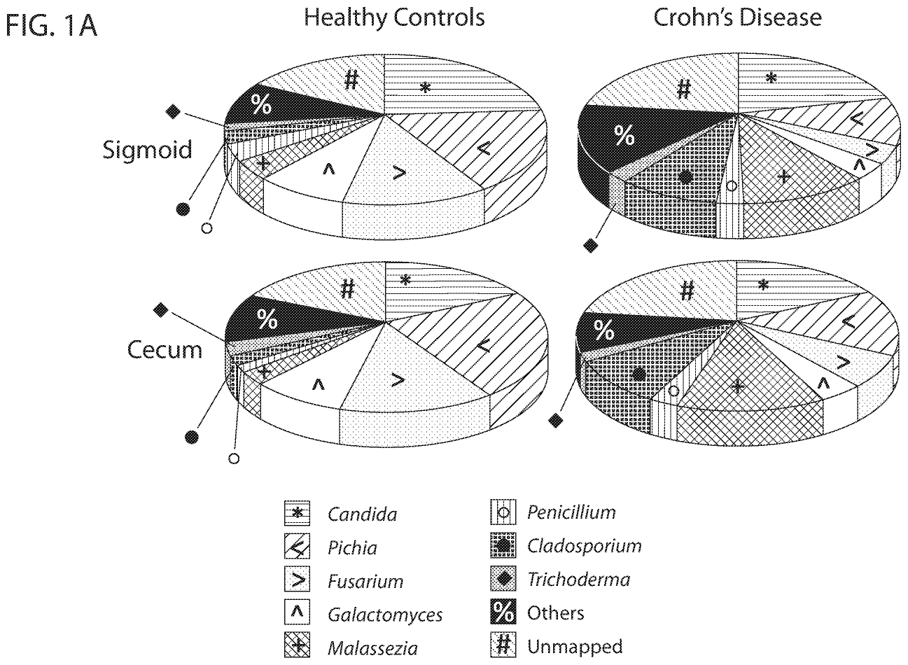

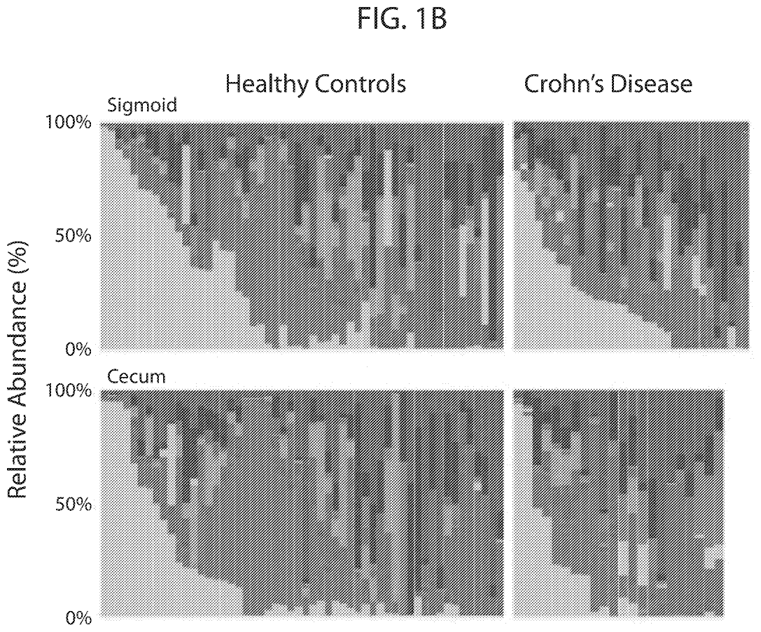

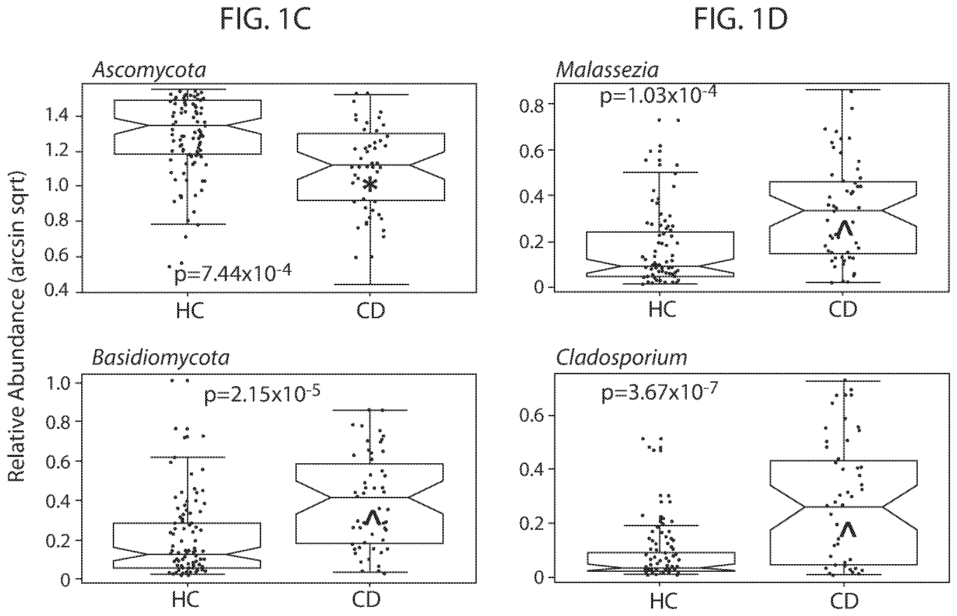

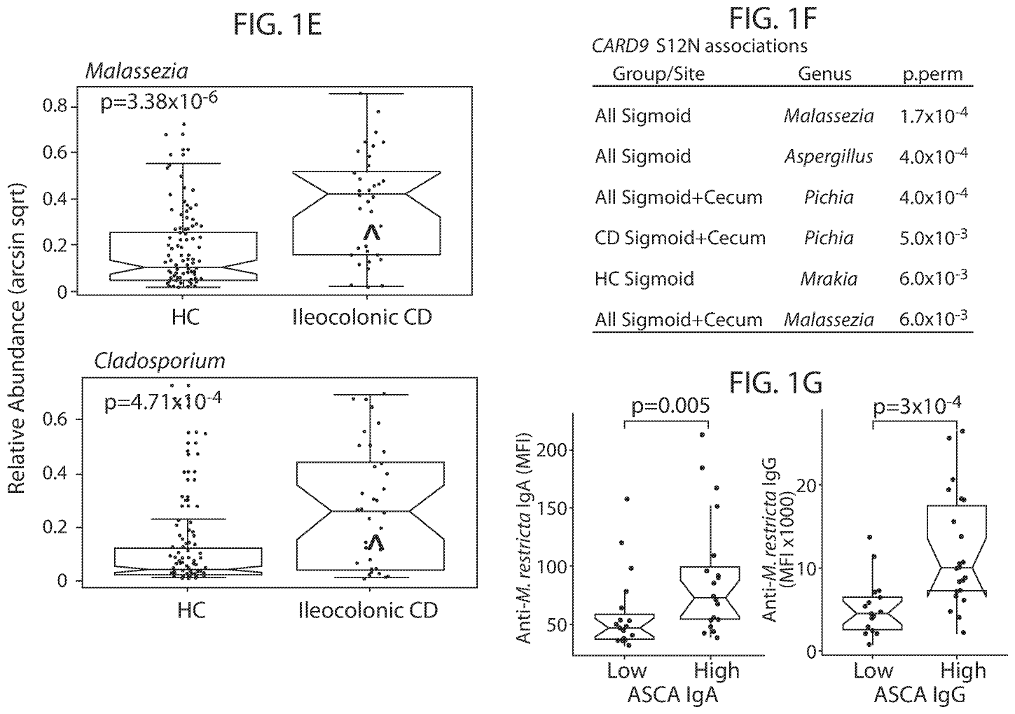

Overall, of the two major fungal phyla, Ascomycota were substantially more common than Basidiomycota in all groups (FIG. 5D). 70 genera of fungi were identified, with the most prevalent 8 genera accounting for more than 60% of the sequences (FIGS. 1A and 1B). Candida and Pichia were the most highly detected, although there was considerable heterogeneity, even among non-diseased control samples. While many samples were dominated by Candida, other groups of samples were dominated by Pichia or Fusarium. In non-diseased control samples (sigmoid/cecum), almost half were dominated by a single genus (48%/48%), while in the Crohn's disease samples, this number fell to less than a quarter (15%/23%).

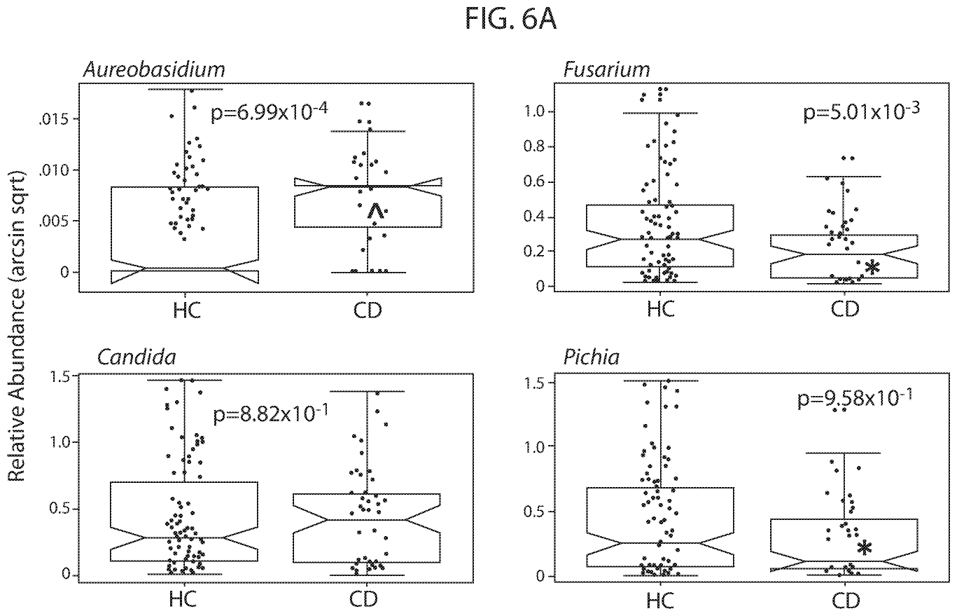

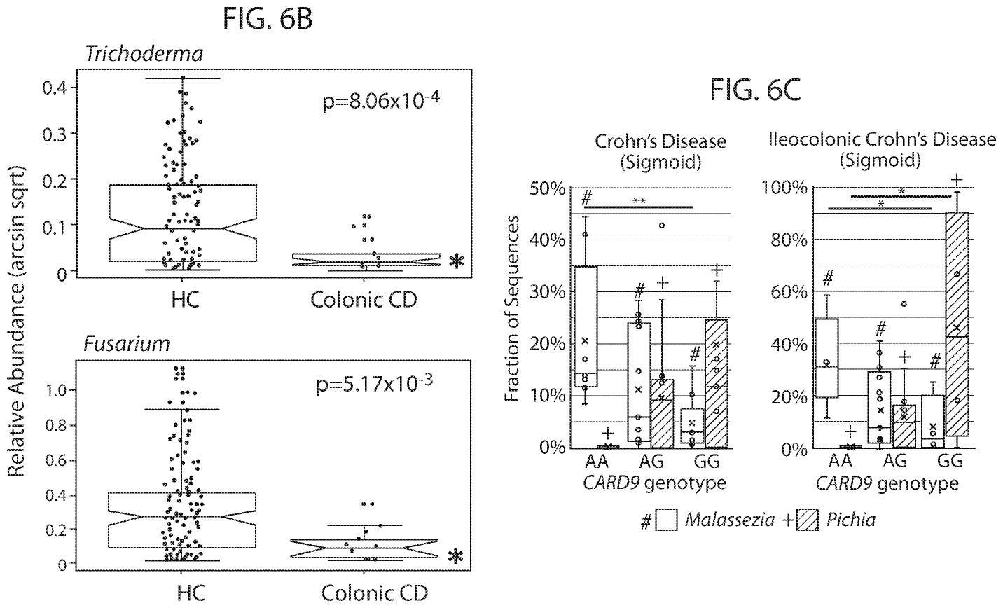

To determine whether there are disease-associated alterations in fungi the inventors applied the MaAsLin (Multivariate Association with Linear Models) method. This is a multivariate statistical framework specifically designed to be used to discover associations between clinical metadata (here being gender, age, diagnosis, and site) and microbial community abundance. It was observed that CD samples are associated with a significant loss of Ascomycota and an associated gain in Basidiomycota (FIG. 1C). Specifically, several fungal genera (Malassezia, Cladosporium, Aureobasidium, and Fusarium) are associated with CD (FIG. 1D and FIG. 6A). The first three are more common in samples taken from patients with Crohn's disease and are rarely found in samples from non-diseased controls. A “control” refers to an individual, or group of individuals, who do not suffer from the inflammatory bowel disease. Malassezia (identified as 65% M. restricta and 34% M. globosa) is responsible for the increase in basidiomycetes in CD patients. Fusarium, an ascomycete, is significantly reduced in CD patients. Other ascomycete fungi commonly of interest such as Candida and Pichia were not distributed unequally between samples from non-diseased controls or patients with Crohn's disease. CD is characterized by diverse clinical characteristics including the primary location of the disease. CD-associated increases in detection of Malassezia and Cladosporium were mainly in patients with ileocolonic disease (FIG. 1E), while the decrease in Fusarium was mainly in patients with colonic disease (FIG. 6B).

The Crohn's disease risk variant of CARD9 is a non-synonymous single nucleotide polymorphism (SNP) leading to a serine to asparagine change at codon 12 (S12N) that may alter the function of the protein. Malassezia spp. was the most strongly linked to CARD9 (FIG. 1F). In sigmoid colon of CD patients, Malassezia spp. was increasingly present as the number of S12N alleles increased, while Pichia was nearly absent in patients homozygous for the S12N risk allele (AA, FIG. 6C). The inventors investigated whether patient sera with high ASCA reactivity also recognize Malassezia restricta and found that ASCA-high patient sera are substantially more reactive against M. restricta than ASCA-low sera (FIG. 1G). Without being bound to any particular theory, the data support a specific link between CD, CARD9, and Malassezia.

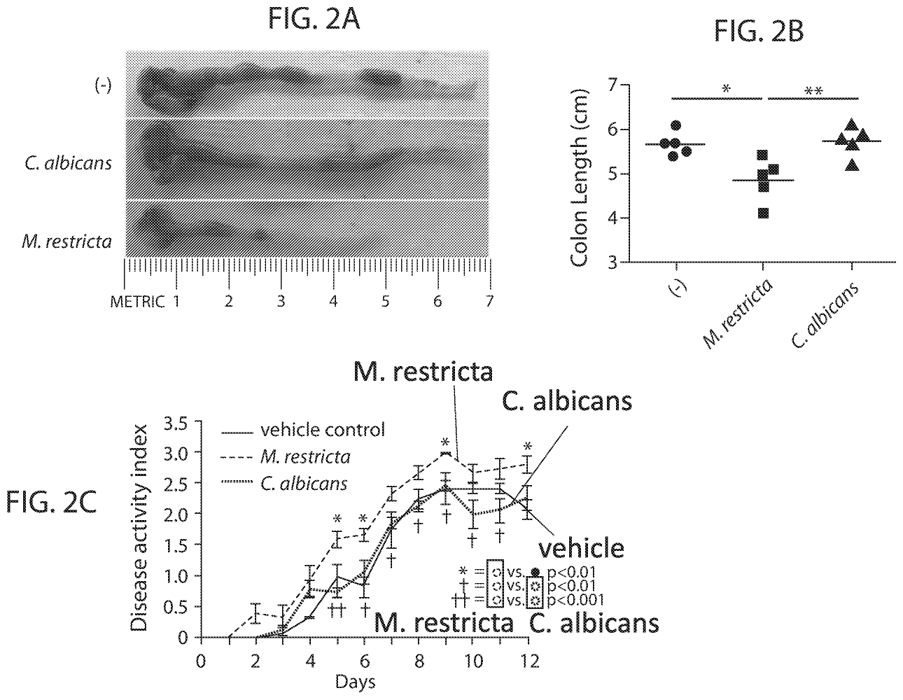

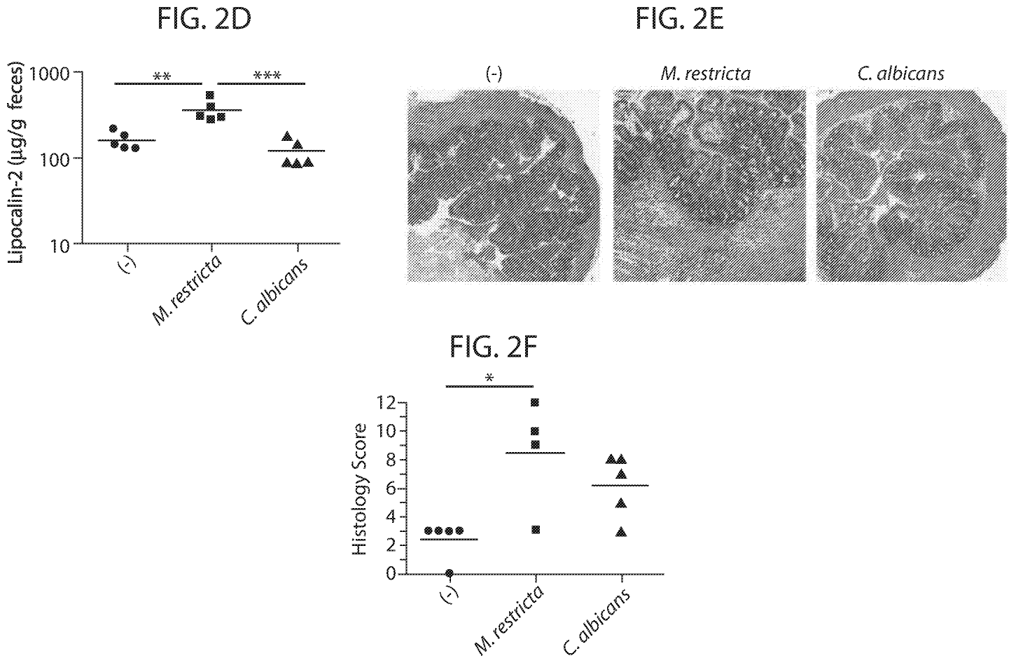

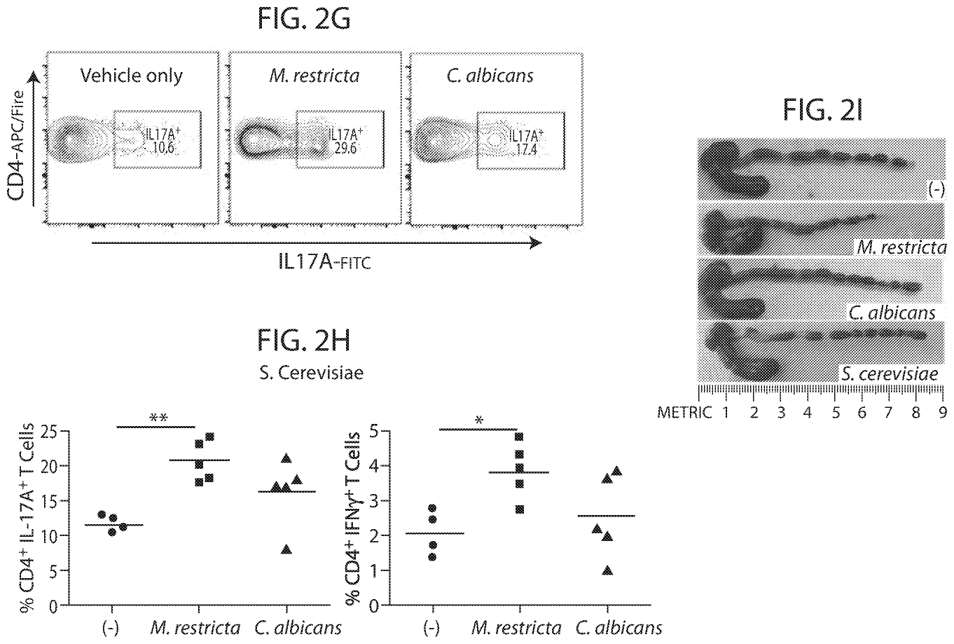

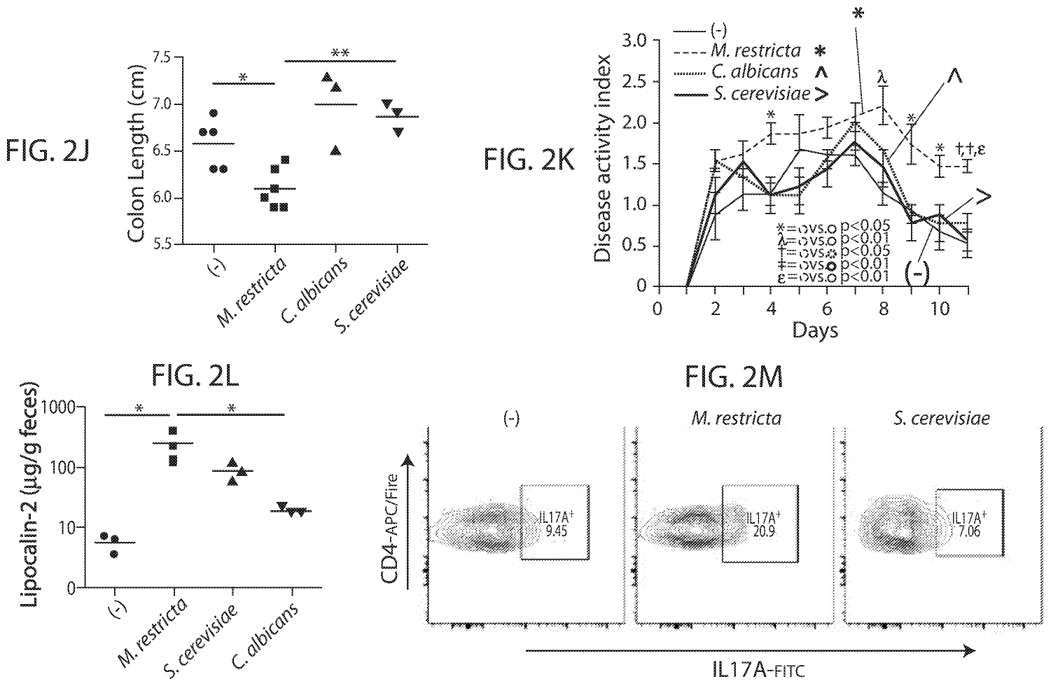

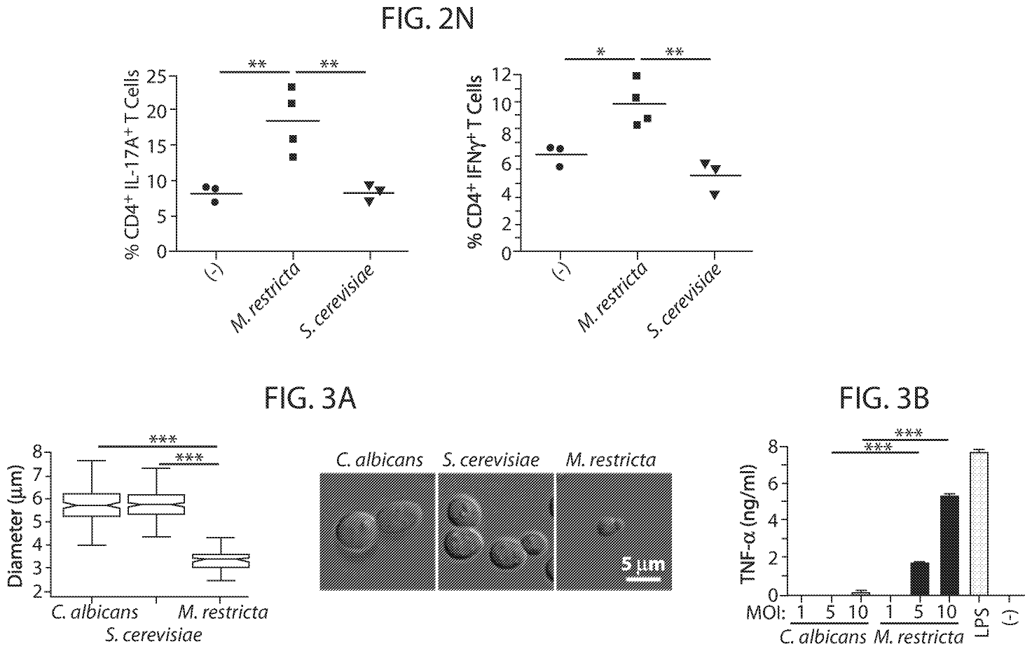

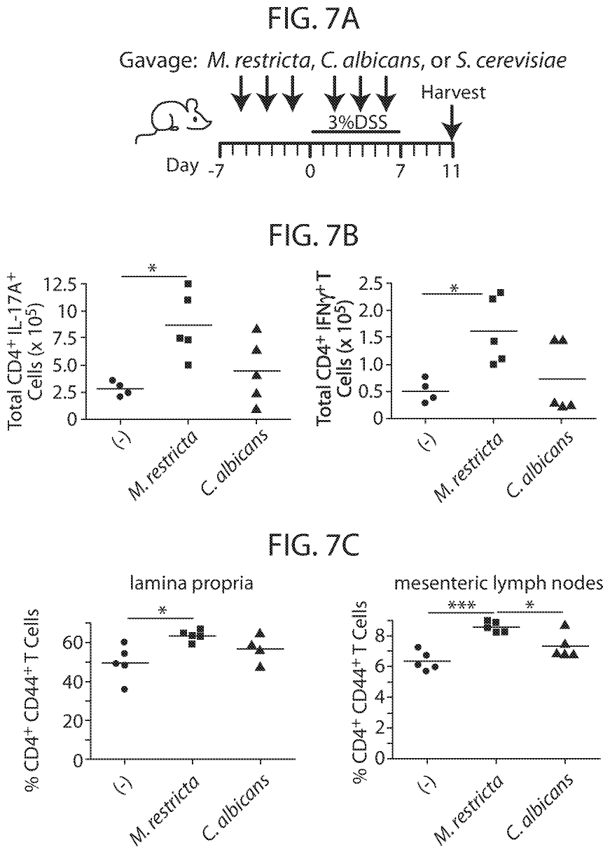

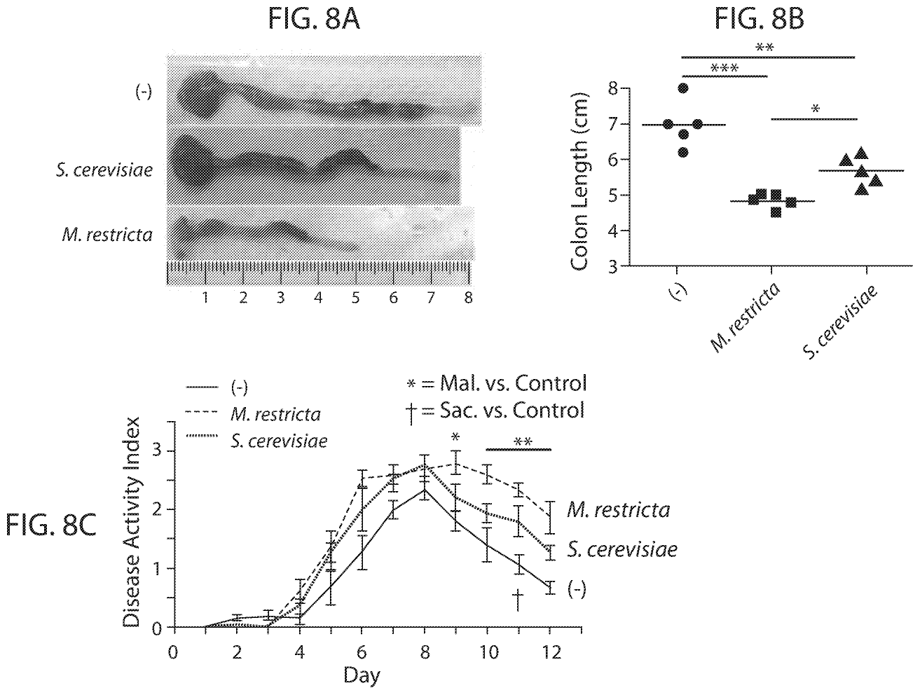

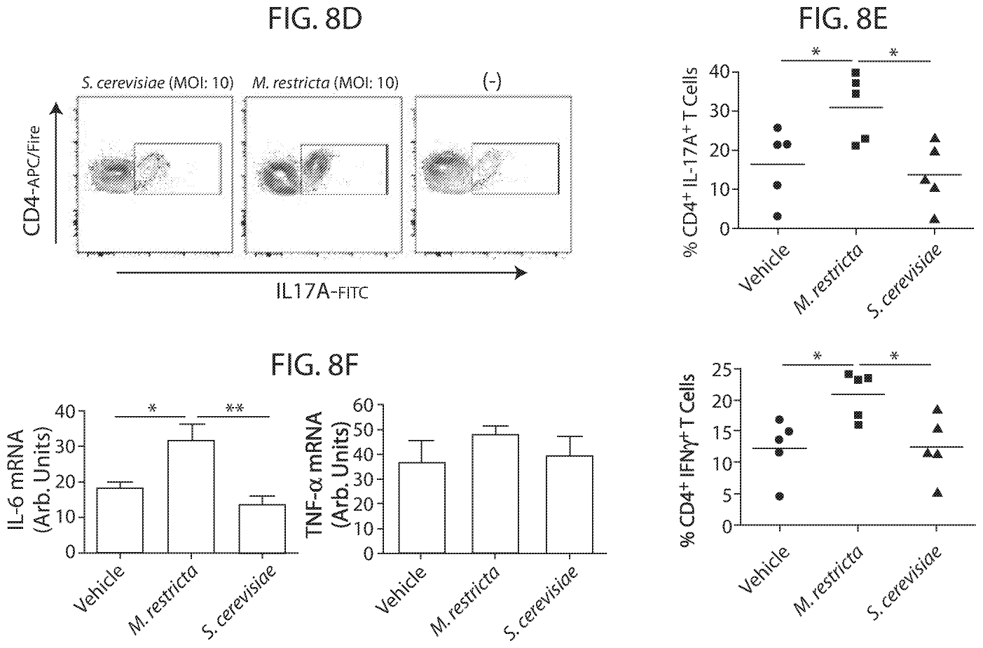

Malassezia restricta is a common fungus that is a natural commensal colonizer of the skin of many animals including humans. It can grow aerobically as well as anaerobically and requires long chain fatty acids for growth (and is thus commonly associated with oilier regions of the skin). Diseases associated with Malassezia spp. range from benign (dandruff) to life-threatening sepsis (typically associated with indwelling catheters). To experimentally determine whether M. restricta might exacerbate colitis, specific pathogen-free (SPF) mice were treated by oral gavage with M. restricta and examined its effect on DSS-induced colitis (FIG. 7A). Oral gavage with M. restricta by itself had no apparent adverse effects, but it promoted more severe DSS-induced colitis as measured by a shortening of the colon (FIG. 2A, B), a worsening of disease activity (FIG. 2C), increased lipocalin-2 levels in the fecal specimens (FIG. 2D), and more severe intestinal inflammation characterized by increased mucosal erosion, crypt destruction and inflammatory cell infiltration in the colon (FIG. 2E, F). Consistent with the histology, restimulation of colonic Lamina propria T cells revealed stronger production of IL-17A- and IFN-γ-producing CD4+ cells which correlated with higher numbers of inflammatory Th1 and Th17 cells (FIG. 2G, 2H and FIG. 7B). Further, an increase in activated CD4+ T cells in the Lamina propria and mesenteric lymph nodes (FIG. 7C) was observed. The effects of M. restricta were consistently more pronounced than C. albicans. A comparison of the effects of S. cerevisiae and the effects of M. restricta showed that M. restrica promoted more severe disease compared to S. cerevisiae (FIG. 8).

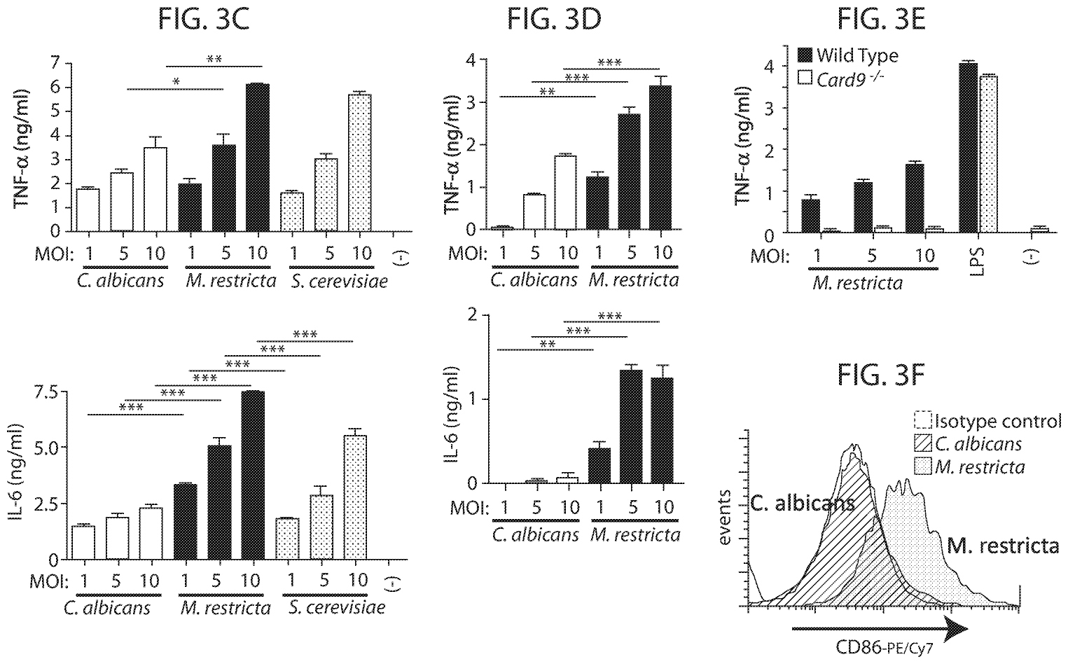

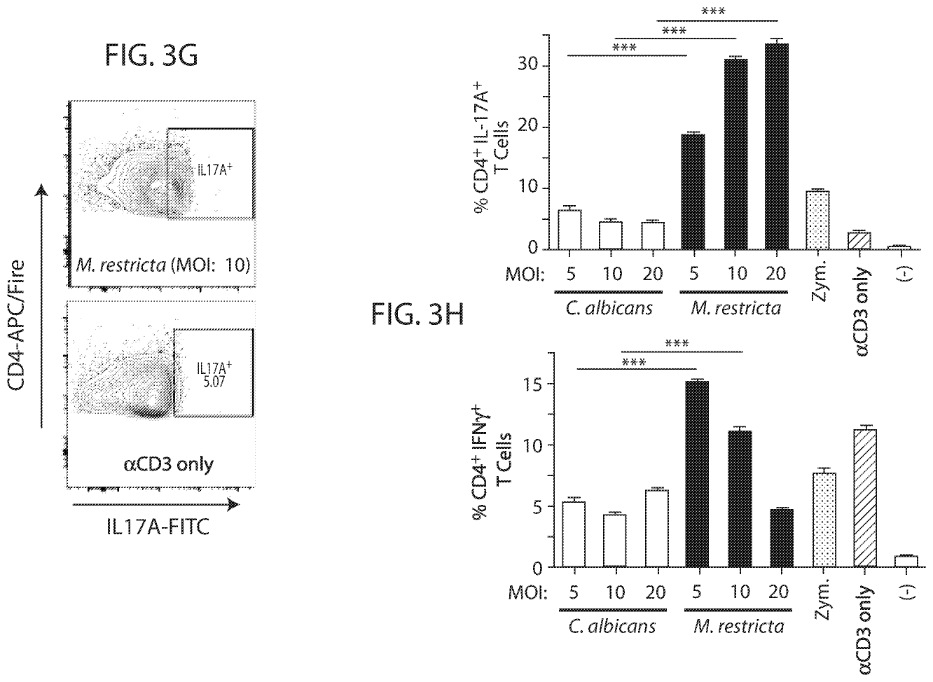

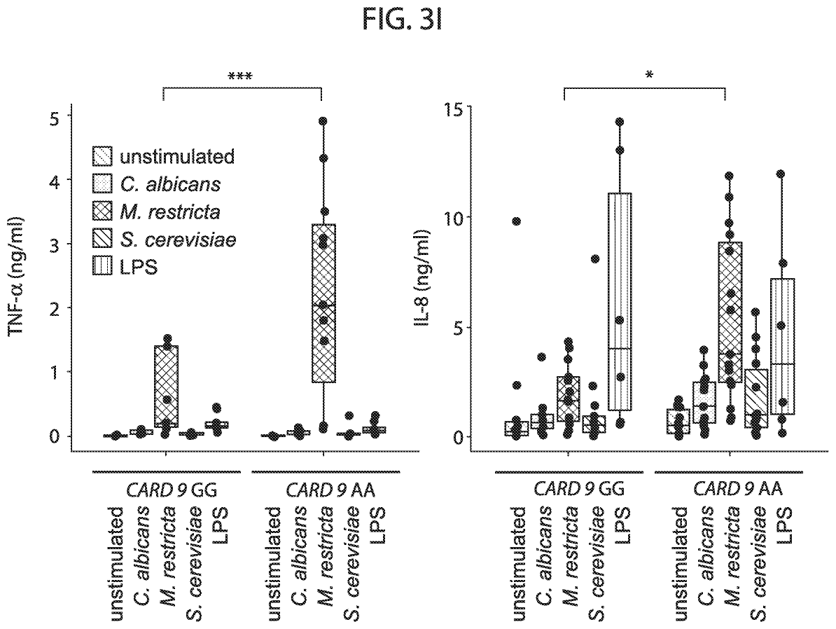

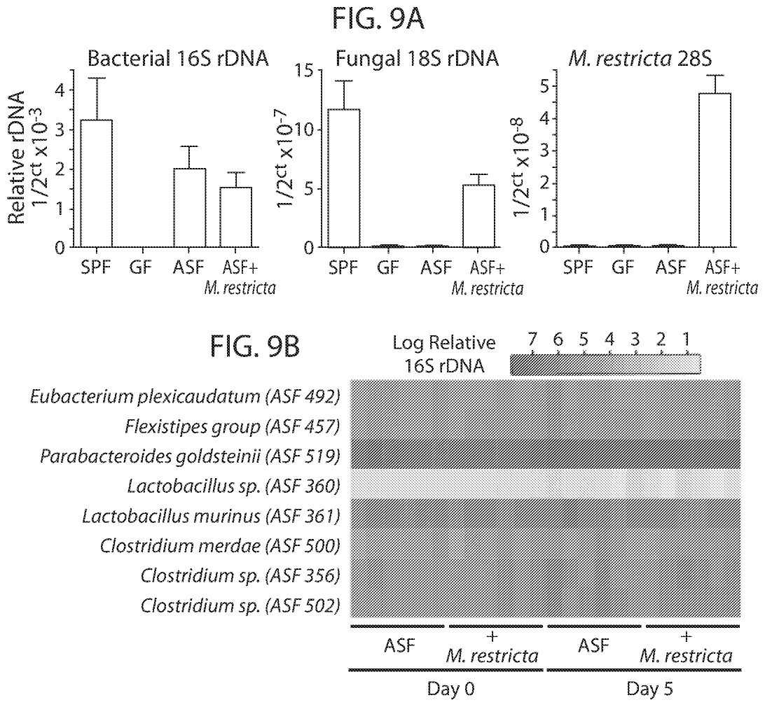

Mechanistically, M. restricta might exacerbate disease directly, or its presence might alter other bacterial or fungal members of the microbiota to exacerbate disease. To better understand whether increased levels of M. restricta alone are sufficient to exacerbate colitis, germ-free mice colonized with altered Schaedler flora (ASF) were utilized. Being colonized with a defined set of 8 bacteria, ASF mice are healthier than germ-free mice, have more mature immune systems, and are fungal-free (FIG. 9A). Oral gavage with M. restricta makes M. restricta the only fungus present, and the inventors found that this does not alter relative levels of any of the ASF bacteria (FIG. 9B). Without being bound to any particular theory, as in SPF mice, M. restricta exacerbated DSS-induced colitis in ASF mice (FIG. 2I-N) suggesting that M. restricta is sufficient to directly exacerbate disease. Although M. restricta is substantially smaller than C. albicans or S. cerevisiae yeasts (FIG. 3A), it generally evokes a stronger pro-inflammatory response from human monocyte-derived dendritic cells (FIG. 3B) and mouse bone marrow-derived dendritic cells and macrophages (FIG. 3C, D).

Malassezia pachydermatis and Malassezia furfur are recognized by Dectin-2 and Mincle; receptors that signal through CARD9, the inflammatory response to M. restricta was highly dependent on CARD9 (FIG. 3E). M. restricta is also particularly potent at inducing expression of co-stimulatory molecules on mouse dendritic cells (FIG. 3F) and, when co-cultured with naïve T cells and anti-CD3a antibodies, these cells induce proliferation and Th1/Th17 polarization more potently than C. albicans yeast (FIG. 3G, H).

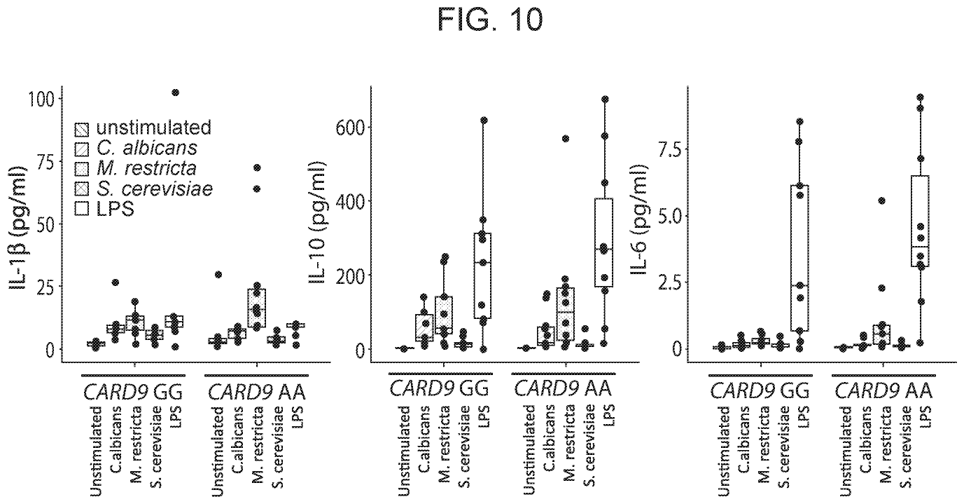

To investigate the consequences of the IBD-associated CARD9 S12N polymorphism on host responses to M. restricta, human peripheral blood monocyte-derived dendritic cells from non-diseased (non-diseased) donors homozygous for the S (GG) or N (AA) alleles were generated and stimulated with fungi. AA dendritic cells produced significantly more TNF-α and IL-8 in response to M. restricta than GG dendritic cells (FIG. 3I and Table 2). Revealing increased inflammatory cytokine production by Malassezia-stimulated CARD9 S12N homozygous cells. Other cytokines including IL-1β, IL-10, and IL-6 were unaffected (FIG. 10), without being bound by any particular theory, suggesting that making the analogous S12N polymorphism in mice alters some, but not all CARD9-dependent signaling.

TABLE 2
Tukey multiple comparisons output for TNF-α and IL-8.
Tukey multiple comparisons of means
(95% family-wise confidence level)

| | diff | lwr | upr | p adj |
|---|---|---|---|---|
| TNF-α Comparison | | | | |
| CARD9 GG:AA Candida | −2.85 | -817 | 811.5 | 1.00 |
| CARD9 GG:AA Malassezia | −1610 | −2468 | −752 | 1E−06 |
| CARD9 GG:AA Saccharomyces | −21.0 | −875 | 833.2 | 1.00 |
| CARD9 GG:AA LPS | 87.14 | −790 | 964.7 | 0.99 |
| IL-8 Comparison | | | | |
| CARD9 GG:AA Candida | −0.7 | −3.74 | 2.34 | 1.00 |
| CARD9 GG:AA Malassezia | −3.5 | −6.55 | −0.46 | 0.01 |
| CARD9 GG:AA Sarcharomyces | −0.58 | −3.67 | 2.51 | 1.00 |
| CARD9 GG:AA LPS | 1.55 | −3.17 | 6.28 | 0.98 |

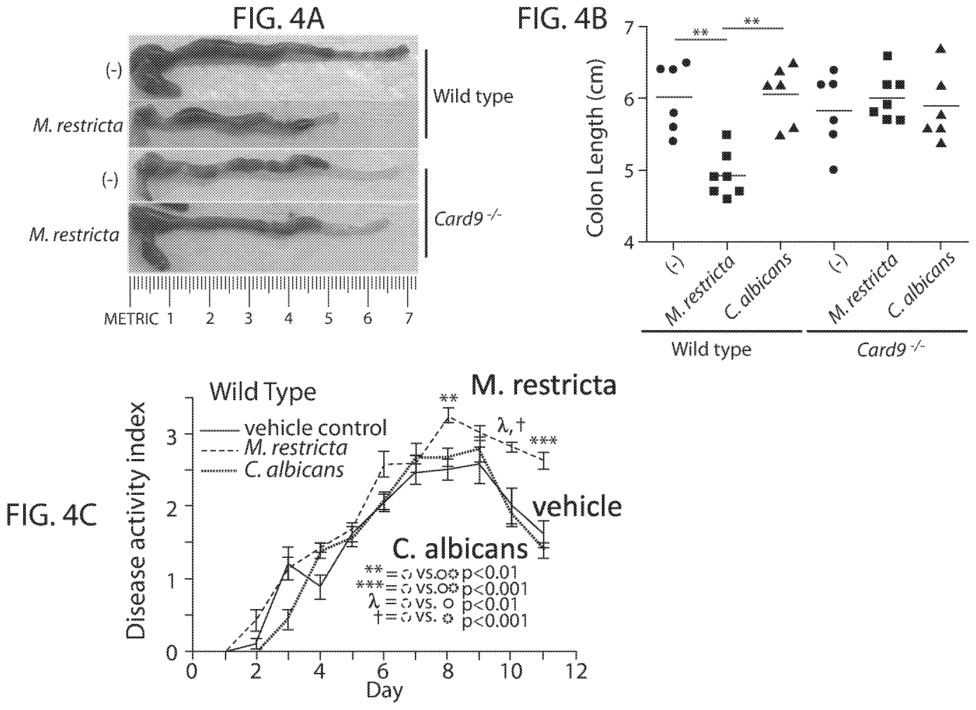

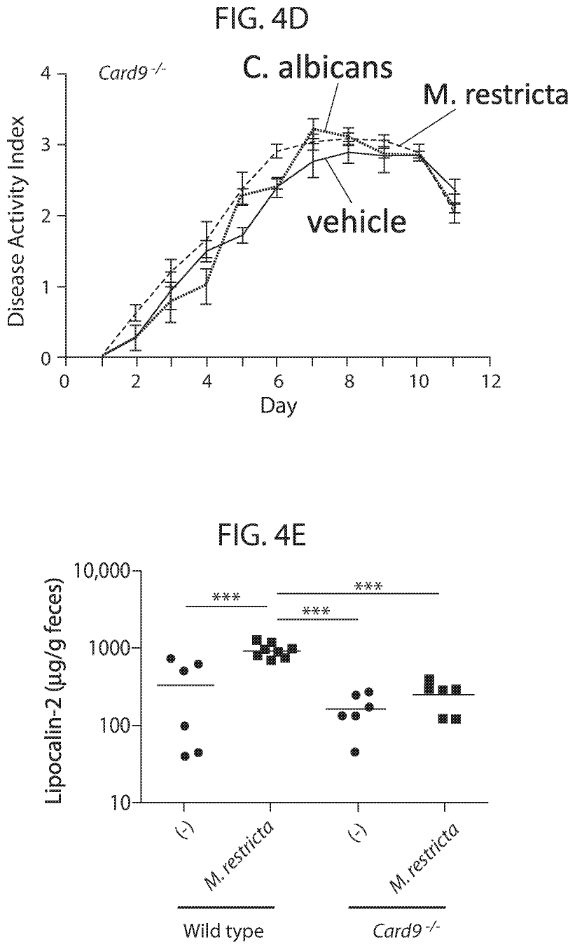

The data suggest that M. restricta exacerbates colitis directly by stimulating inflammatory responses via CARD9. The inventors observed that loss of CARD9-mediated signaling prevented M. restricta from exacerbating disease as measured by reduced shortening of the colon (FIG. 4A, B), amelioration of disease activity (FIG. 4C, D), and decreased lipocalin-2 levels in the fecal specimens (FIG. 4E). Without being bound by any particular theory, the data suggest that changes in intestinal fungi and host responses to intestinal fungi may contribute to disease in a subset of patients with Crohn's disease. The functional consequences of the association of Candida spp. in the stool with CD, are not yet clear. Without being bound to any particular theory, the data suggest that intestinal colonization with M. restricta, a common commensal fungus found typically on the skin, may increase disease severity. Without being bound by any particular theory, the findings suggest that a precision approach therapeutically targeting specific members of the fungal microbiota in certain individuals should be explored.

### Materials and Methods

Sample Collection

A previously-assembled and newly-expanded cohort of non-diseased individuals undergoing screening colonoscopy and Crohn's disease patients undergoing colonoscopy for clinical reasons was examined in accord with human subject protocols approved by the institutional review board of Cedars-Sinai Medical Center including written informed consent of each subject to participate in the study. All enrolled subjects were prepared for colonoscopy by taking Golytely® the day before the procedure. Mucosal lavage samples representing the mucosal luminal interface were collected from different intestinal regions. All samples were collected from non-inflamed intestinal regions, which excluded the potential influence of active inflammation on the mucosal microbiota as much as possible. Subjects' metadata, including diagnosis, location of disease, gender, age, and colon regions sampled, were recorded.

High-Throughput 16S and ITS1 Sequencing.

During DNA isolation and sequencing, investigators were blinded to sample identities. DNA was extracted from 166 samples using the PowerSoil DNA Isolation Kit (Mo Bio Laboratories) including a 30-second bead-beating in a Mini-Beadbeater-16 (BioSpec). Fungal ITS1 amplicons were generated in 20 μL PCR reactions using 3 μL of each sample with 35 cycles using PHUSION DNA Polymerase (New England BioLabs) at an annealing temperature of 56.1° C. using the primers ITS1F (CTTGGTCATITAGAGGAAGTAA (SEQ ID NO:22)) and ITS2 (GCTGCGTTCTTCATCGATGC (SEQ ID NO:23)) incorporating an extra 6 bases at the 5′ end as sample-specific bar codes. Resultant ITS amplicons were purified using Agencourt AmPure Magnetic Beads (Beckman Coulter), resuspended in 20 μL of nuclease-free water, and quantified using a Qubit fluorometer. Amplicons were further qualified using the DNA 1000 assay on the Agilent Bioanalyzer (Agilent Technologies). To generate Illumina TruSeq libraries, 8 barcoded libraries were pooled per TruSeq index to be used, and TruSeq adapters were ligated to the amplicons via the DNA Nano LT kit (Illumina). Final libraries were quantified with Qubit's high sensitivity dsDNA assay, and qualitied with the Agilent Bioanalyzer DNA 1000 assay.

Sequencing was performed on the Illumina MiSeq platform (Illumina) with paired end 250 bp sequencing chemistry, giving the possibility to obtain relatively long overlapping reads with less than 1% error rates. Fungal ITS1 libraries were clonally amplified directly onto paired end flowcells and sequenced with standard Illumina sequencing primers according to manufacturer's instructions. Raw data processing and run demultiplexing was performed using on-instrument analytics as per manufacture recommendations. Sequence reads from this study are available from the Sequence Read Archive under the project ID “PRJNA306760”.

Data Processing

For analysis of ITS1 sequence data, raw FASTQ data were filtered to enrich for high quality reads including removing the adapter sequence by cutadapt v1.4.1 (D. Li et al., Gastroenterology 151, 724-732 (2016), demultiplexing and truncating reads not having an average quality score of 20 (Q20) over a 3 base pair sliding window, removing any reads that do not contain the proximal primer sequence or any reads containing a single N (unknown base) by a custom script. Filtered pair-end reads were then merged with overlap into single reads using SeqPrep v1.0 wrapped by QIIME v1.6 with default settings.

The processed high-quality reads were firstly aligned to previously observed host sequences (including rRNA, olfactory receptor and uncharacterized genes in human and mouse) to deplete potential contamination, then operational taxonomic unit (OTU) were picked by aligning filtered reads to the Targeted Host Fungi (THF) custom fungal ITS database (version 1.6) (G. Hoarau et al., MBio 7, (2016), using BLAST v2.2.22 in the QIIME v1.6 wrapper with an identity percentage ≥97%. Only less than 0.36% reference sequences in THF database could not be annotated down to genus level (and assigned as “Unidentified” for genus name). OTUs with average relative abundance >0.00001 were considered to be present as well as compiled into genera for downstream analysis.

Bioinformatic Analysis

While intestinal disease can be exacerbated or ameliorated by organisms that do not change in overall abundance, the inventors focused the first line of analysis on evaluating whether specific mucosa-associated fungi are enriched or depleted in the context of Crohn's Disease. The alpha diversity measures of ITS OTUs were calculated including Shannon's diversity index, inverse Simpson index, observed OTUs with R package phyloseq v1.13.3 on the unfiltered OTU table to better estimate the diversity. The filtered OTU table was used for all downstream analysis. Two-sided Wilcoxon test was used to examine the differences in alpha diversity indices. The pair-wise differential abundance analysis was conducted by using linear discriminant analysis with LEfSe v1.0.7 at default settings. The multivariate association analysis was conducted by using MaAsLin v0.0.3 with minimum OTU prevalence of 0.05, minimum abundance of 0.001 and FDR-corrected p-value cutoff of 0.10, on the variables of interest: gender, age, body site and diagnosis.

CARD9 S12N allele data were acquired using ImmunoChip. Genetic association between the CARD9 S12N allele and fungi was performed in the generalized linear model (GLM) framework, with adjustment for Principle Components (PCs) from population stratification analysis. Permutation test was performed to control for false-positives that can be caused by the skewness of the fungal data. The corrected significance threshold for this analysis is 0.05/75=6.7×10⁻⁴.

Antisera Analyses

Human serum was collected from CD patients (n=18-22 per group) after informed consent by the Cedars-Sinai Medical Center MIRIAD Biobank in accordance with Cedars-Sinai Medical Center Institutional Review Board procedures. ASCA-IgA and ASCA-IgG level determination was made by the Cedars-Sinai Medical Center MIRIAD Biobank using the method described by Landers et al. (G. Liguori et al., J. Crohns Colitis 10, 296-305 (2016). M. restricta was grown as described below and washed two times with sterile 1×PBS. Five million live yeasts were plated per well onto 96 well U-bottom plates and stained with human serum diluted in FACS buffer at 1:100 in 100 μL. Primary staining was done for 30 minutes at room temperature followed by washing with 150 μL of FACS buffer. Samples were then stained with Alexa Fluor-488 conjugated goat anti-human IgA (Southern Biotech) and Alexa Fluor-647 conjugated goat anti-human IgG (Southern Biotech) in FACS buffer at a 1:300 dilution for 30 minutes at room temperature followed by washing. Stained cells were fixed in 2% paraformaldehyde, washed and analyzed on a BD LSR2 flow cytometer (BD Biosciences). Flow cytometry analysis was performed using FlowJo software (TreeStar). Flow cytometry data was reported as mean fluorescence intensity.

Mice

C57BL/6 mice 8-10 weeks of age were used for most experiments and purchased from Jackson Laboratories (Bar Harbor, Me.). Bone marrow-derived dendritic cells and macrophages were prepared from C57BL/6 mice from Jackson Laboratories (Bar Harbor, Me.) or from Card9 knockout mice maintained under specific pathogen-free conditions. Germ-free mice were maintained under sterile conditions in flexible film isolators in the Cedars-Sinai Medical Center animal facility. A colony of Altered Schaedler Flora (ASF)-colonized mice was generated by colonizing germ-free mice with ASF stool (Taconic, Germantown, N.Y.). Colonization was verified by PCR analysis of the ASF component bacteria, and absence of fungi was assessed by PCR as previously described (L. Jostins et al., Nature 491, 119-124 (2012). For in vitro experiments, male and female mice were used, and no differences were noted. For in vivo experiments, female mice were used unless otherwise stated. Mice were housed in specific pathogen-free conditions in the Cedars-Sinai animal facility, and all animal experiments were conducted according to Cedars-Sinai Medical Center Institutional Animal Care and Use Committee guidelines.

Preparation of Yeasts

Candida albicans (ATCC 90028) and Saccharomyces cerevisiae (ATCC 201388) yeasts were grown with shaking overnight at 37° C. in Sabouraud dextran broth (SDB). Malassezia restricta (clinical isolate MYA-4611) was grown statically for 3 days at 30° C. in modified Dixon broth (mDixon) supplemented with glycerol monostearate. For in vitro experiments yeasts were fixed in 2% paraformaldehyde at room temperature for 1 hour then washed 3 times with cell culture media and counted. For in vivo experiments fungi were grown as described above washed 3 times with 0.2 M sodium bicarbonate and resuspended at a concentration of 1×10⁹ yeasts/mL of 0.2 M sodium bicarbonate. Mice were dosed with 1×10⁸ yeast cells at the times indicated.

Macrophage and Dendritic Cell Preparation and Stimulation

Mouse bone marrow-derived macrophages and dendritic cells were grown as previously described (M. A. Rivas et al., Nat. Genet. 43, 1066-1073 (2011)). For in vitro assays dendritic cells or macrophages were plated at 400,000 cells per well in 24 well plates and stimulated with indicated doses of fungal cells for 24 hours. Supernatants were collected and used for cytokine measurements by ELISA.

For human monocyte derived dendritic cells, human blood was collected from non-diseased donors after informed consent by the Cedars-Sinai Medical Center MIRIAD Biobank in accordance with Cedars-Sinai Medical Center Institutional Review Board procedures. Peripheral blood mononuclear cells were isolated by Ficoll-Paque Premium gradient (GE Healthcare) and CD14+ monocytes were negatively selected using EasySep™ Human Monocyte Enrichment Kit (Stem Cell Technologies). Purified CD14+ monocytes were cultured in the presence of human GM-CSF and human IL-4 (Peprotech) for 7 days. Differentiated cells were plated at 200,000 cells per well in 48 flat bottom tissue culture treated plates (Corning, Inc., Corning, N.Y.) and stimulated for 24 hours with indicated doses of fungal cells. Supernatants were collected for measuring cytokine production by Meso Scale Diagnostics V-Plex Plus Human Proinflammatory Panel 1 or ELISA (BioLegend).

DSS Colitis

Where indicated mice were supplemented with 3% DSS (MP Biomedicals LLC) in their drinking water for 7 days, for the induction of colitis, followed by 4-5 days of recovery on regular water. For mice administered fungi, the dosing is as described above. Mice were gavaged 3 times before the introduction of DSS water every other day and 3 times every other day following DSS water. Disease activity index was accessed as previously described (I. D. Iliev et al., Science 336, 1314-1317 (2012). Histological analyses were performed by a trained pathologist blinded to the experimental conditions as previously described (L. Jostins et al., Nature 491, 119-124 (2012) and R. Perez de Diego et al., J. Allergy Clin. Immunol. 136, 1139-1149 (2015)).

Fecal Lipocalin-2

As a non-invasive biomarker for intestinal inflammation, the inventors measured fecal lipocalin-2 levels. Mouse fecal pellets were collected in sterile 1.7 ml microcentrifuge tubes and resuspended at 100 mg/mL of sterile PBS with protease inhibitor cocktail (Cell Signaling Technology). Samples were shaken using a bead beater at medium speed for 90 seconds followed by centrifugation. Supernatants were assayed for lipocalin-2 using LEGEND MAX mouse NGAL (Lipocalin-2) ELISA Kit (Biolegend).

Colonic Lymphocyte Lamina propria Isolation and Ex-Vivo Stimulation

Lymphocytes from the colonic Lamina propria were isolated by using the mouse Lamina Propria Dissociation Kit and AutoMacs per the manufacturer's instructions (Miltenyi Biotec). After tissue dissociation the samples were re-suspended in a 42% Percoll gradient solution and layered on top of a 72% Percoll gradient for centrifugation and isolation of single cells. Isolated cells were re-suspended in 1×PBS containing 2% FBS counted and stained with fixable viability dye, Zombie UV followed by Fc blocking and staining with mouse CD4 antibody (BioLegend). After staining, cells were washed and re-suspended in complete media and stimulated for 6 hours using cell stimulation cocktail (eBioscience) in the presence of brefeldin A (BioLegend) and GolgiStop (BD Biosciences). After stimulation, cells were fixed in 2% paraformaldehyde washed and stained for intra-cellular cytokine production using Intracellular Staining Permeabilization Wash Buffer (BioLegend) per the manufacturer's instructions.

Antibody Staining and Flow Cytometry

Single cells preparations were first treated with fixable cell viability dye, Zombie UV followed by TruStain fcX (BioLegend) to block Fc receptors. Fluorophore conjugated antibodies were used as follows: anti-CD4 (clone GK1.5), anti-CD44 (clone IM7), anti-CD62L (clone MEL-14), anti-CD86 (clone GL-1) (BioLegend), anti-IFNγ (clone XMG1.2), anti-IL17A (clone eBiol7B7), anti-CD45 (clone 30-F11) (eBioscience). Stained cells were fixed in 2% paraformaldehyde and analyzed on a BD LSR2 flow cytometer (BD Biosciences). Flow cytometry analysis was performed using FlowJo software (TreeStar).

Mouse Dendritic Cell and CD4+ T Cell Co-Culture

Mouse dendritic cells were generated and plated as described above. Naïve CD4+ T cells were purified from mouse spleen and peripheral lymph nodes using a CD4 Naïve Enrichment Kit (Life Technologies). Prior to adding naïve T cells, dendritic cells were stimulated with indicated fungal cell doses for 3 hours to allow for complete fungal cell internalization. Naïve CD4+ T cells were then added at a ratio of 2:1 with or without anti-CD3ε antibodies (BioLegend) for poly-clonal T cell stimulation. Cells were co-cultured for 5 days and processed for intracellular cytokine production as described above.

Colonic RNA Purification, cDNA Synthesis and qPCR Analysis Colonic sections were collected and homogenized in TRIzol reagent per the manufacturer's instruction.

The aqueous fractions were processed with RNeasy Mini Kit (QIAGEN) for RNA purification. cDNA synthesis was carried out using iScript cDNA Synthesis Kit (BIORAD) per the manufacturer's instructions. Gene expression of colonic samples was performed using iTaq Universal SYBR Green Supermix (BioRad), on an Eppendorf Mastercycler ep realplex2 and realplex 2.2 software. Mouse β-actin was used as the housekeeping gene. Primers used are as follows: TNFα forward primer 5′-TCTCATGCACCACCATCAAGGAC-3′ (SEQ ID NO:24) and reverse primer 5′-TGACCACTCTCCCTITGCAGAAC-3′ (SEQ ID NO:25), il-6 forward primer 5′-ATCCAGTTGCCTTCTTGGGACTG-3′ (SEQ ID NO:26) and reverse primer 5′-TAAGCCTCCGACTTGTGAAGTGG-3′ (SEQ ID NO:27), β-actin forward primer 5′-GGCTGTATTCCCCTCCATCG-3′ (SEQ ID NO:28) and reverse primer 5′-CCAGTTGGTAACAATGCCATGT-3′ (SEQ ID NO:29).

Statistical Analysis

All experiments were conducted with at least triplicate measurements a minimum of two times unless otherwise stated in the text or figure legends. Statistics were measured by Student's t-test or One-Way ANOVA with Tukey's multiple comparison test for significance using GraphPad Prism software or R version 3.4.0. Mann-Whitney U Test performed where data failed tests for normality.

### Example 2

Although it is clear that the gut bacterial microflora plays an important role in influencing inflammatory bowel diseases, very little is known about the role of gut fungi in regulating a non-diseased gut microflora and in contributing to inflammatory disorders. The inventors have recently observed that mice lacking the gene for Dectin-1, an innate immune receptor expressed by macrophages and dendritic cells that is essential for host defense against fungi, are more susceptible to colitis. The inventors have further developed methodology for characterizing the fungal microbial community found in wild type and Dectin-1 deficient mice. The inventors have discovered a strong association between a human polymorphic variant of Dectin-1 and severe ulcerative colitis. Without being bound to any particular theory, the inventors do not see an association between this gene and Crohn's disease. Without being bound to any particular theory, the data lead the inventors to hypothesize that genetic variation in Dectin-1 specifically, and anti-fungal immunity generally, can influence severity of disease in ulcerative colitis patients.

The inventors explore the fungal mycobiome in humans, look at whether the ulcerative colitis-associated Dectin-1 polymorphism affects expression or function of the receptor in genotyped patient populations, and determine whether the polymorphism is associated with alteration in the colonic fungal microflora. The studies could provide a strong rationale for anti-fungal therapy tailored to genetically susceptible ulcerative colitis patients.

The inventors set out to define the fungal mycobiome in colonic mucosal washes from IBD patients with and without Dectin-1 risk polymorphism and define the functional consequences of Dectin-1 risk polymorphisms on Dectin-1 expression and function.

The inventors collected sets of matching colonic washes, and colonic biopsies from IBD patients having the Dectin-1 risk haplotype or not (45 patients per group) and amplified fungal rDNA from a subset of these samples and subject them to massively parallel sequencing on the Illumina Next-Generation platform. The inventors defined fungal microbiomes in the samples and assessed whether alterations in the microbiomes are associated with Dectin-1 genotype or disease severity.

In addition, the inventors collected blood from non-diseased donors having the Dectin-1 risk haplotype or not, generate macrophages, and assess Dectin-1 expression and function. The inventor further isolated genomic DNA and sequenced the gene associated with severe ulcerative colitis to try to more specifically identify the alteration conferring risk. If a more specific genetic change was found, the inventors further characterized Dectin-1 expression and function associated with this change.

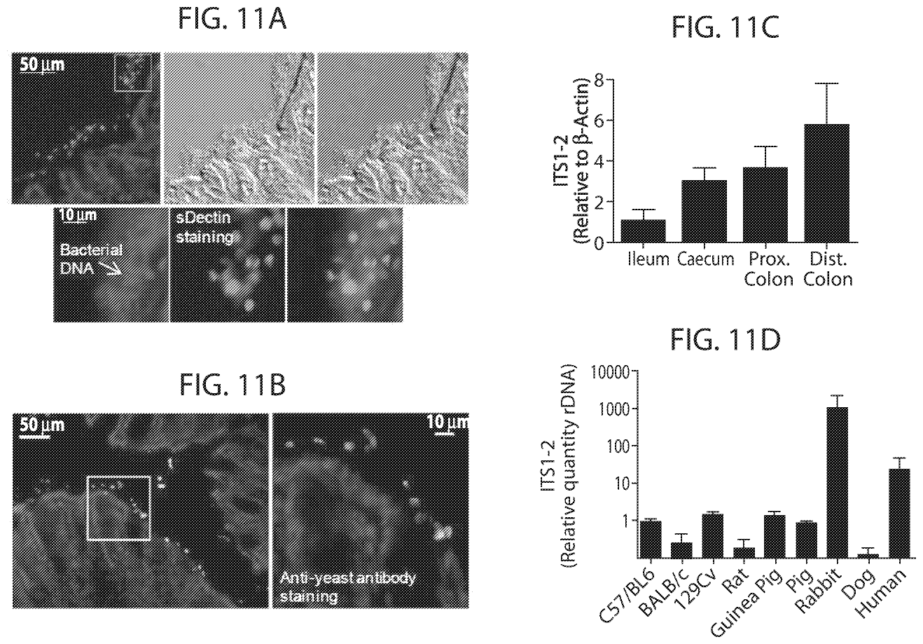

Fungal microflora: Gut fungi have been relatively overlooked, but are not an insignificant portion of the total gut microflora. Given that fungi are on average 50 times larger than bacteria, as much as half of the microbial biomass associated directly with the mucosa in specific regions of the intestines may be fungal. In preliminary studies by the inventor, fluorescence in situ hybridization (FISH) analysis of murine cecum and colon revealed that fungi are located in the mucus layer, in close vicinity with intestinal epithelial cells and share a similar niche with commensal bacteria (FIGS. 11A&11B). The fungi in the mucus showed spherical and filamentous morphologies, with spherical forms being more common. The inventors also examined the distribution of fungi in the murine gastrointestinal tract by PCR and detected fungal rDNA throughout the intestines with highest densities in the terminal colon of C57BL/6 mice (FIG. 11C) as well as in rat, guinea pig, rabbit, pig, dog and human fecal specimens (FIG. 11D), without being bound to any particular theory, suggesting that commensal fungi contribute to the intestinal microbial community in many species.

Fungi and human inflammatory bowel disease (IBD): Anti-Saccharomyces cerevisiae antibodies (ASCA) are directed against yeast cell wall mannan. ASCA was the first biomarker that was capable of identifying the majority of patients with Crohn's disease and it remains the single most robust biomarker for Crohn's disease. 50-60% of patients with Crohn's disease have circulating antibodies to fungi (ASCA) while these antibodies are rare in non-diseased individuals. The “ASCA” terminology is a bit misleading since mannan is part of the cell wall of nearly all fungi and the antibodies may or may not have been generated by exposure specifically to S. cerevisiae. More recently, antibodies to glucan and chitin (other fungal wall components) have also been measured in serum from IBD patients. It is not yet clear whether the appearance of anti-fungal antibodies might indicate that altered immunity to fungi is a cause of disease or whether the intestinal inflammation occurs in such a way that it specifically promotes subsequent exposure to fungi. Alterations in the types of fungi found in the intestines of IBD patients have been reported, but again, whether this is causative or correlative is not clear.

Dectin-1, an innate immune receptor that recognizes fungi. Dectin-1 is a type II transmembrane protein with a short amino-terminal cytoplasmic tail and a single extracellular C-type lectin domain. Signaling by Dectin-1 triggers production of reactive oxygen species and phagocytosis. In addition, Dectin-1 activates inflammatory cytokine production through a signaling complex of CARD9, Bcl10, & Malt1. Dectin-1 is a C-type lectin receptor that recognizes β-1,3-glucans found in the cell walls of nearly all fungi. Primarily expressed by myeloid cells, such as macrophages, dendritic cells (DC), and neutrophils, Dectin-1 is the main receptor for phagocytosis of fungal pathogens. In addition, Dectin-1 activation triggers an oxidative burst, degranulation, killing, and influences production of many cytokines and chemokines that are necessary for developing an effective immune response. Dectin-1 signaling requires a signaling adaptor molecule called CARD9. Genetic variants of CARD9 have been identified by several groups to be strongly associated with Crohn's disease and ulcerative colitis.

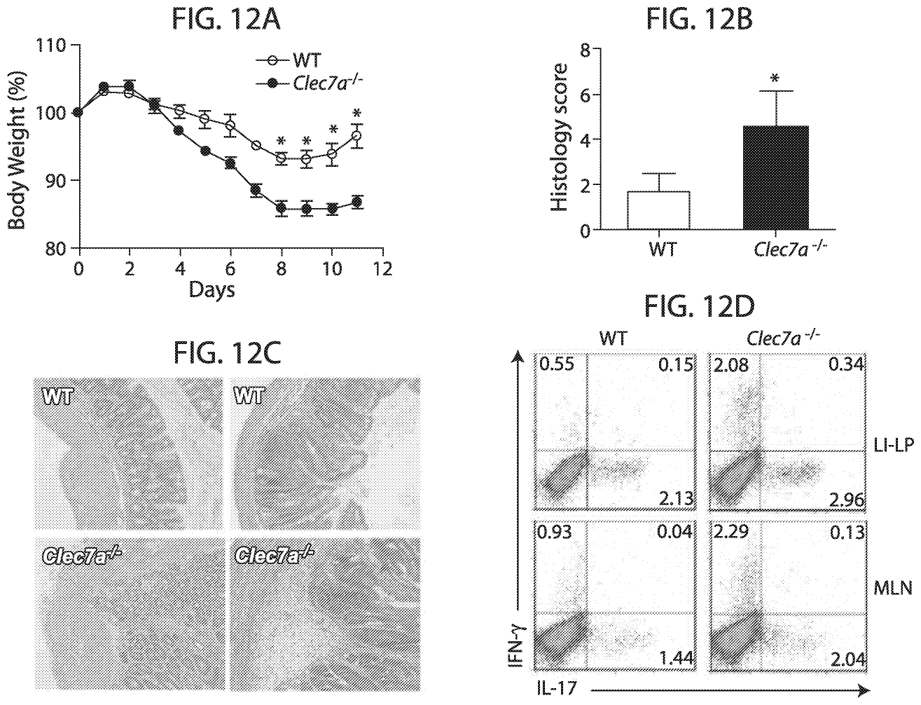

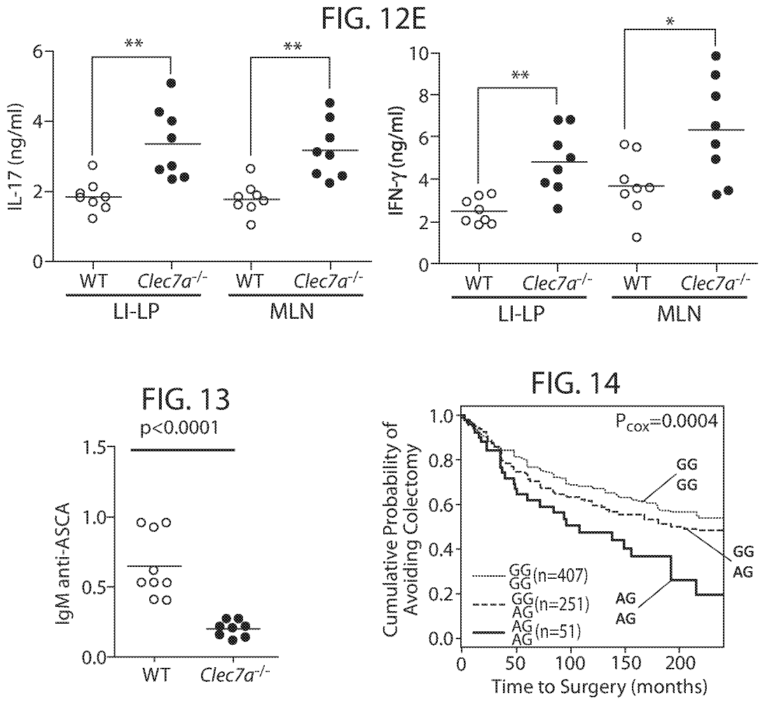

DSS colitis in Dectin-1-deficient mice: The inventors examined whether defective immune recognition of fungi could have an impact on colitis and looked at the effects of inducing acute colitis in mice lacking Dectin-1 (Clec7a−/−). The inventors co-housed wild type and Clec7a−/− mice for two weeks before inducing acute colitis by administration of 3% DSS in the drinking water. The inventors found that after DSS treatment, Clec7a−/− mice experienced increased weight loss (FIG. 12A) and displayed altered histology characterized by increased mucosal erosion, crypt destruction and inflammatory cell infiltration in the colon (FIG. 12B, 12C) as compared to their WT controls. Consistent with the histology, the inventors detected augmented production of IFN-γ and IL-17 in mesenteric lymph nodes (MLNs) and colons from Clec7a−/− mice (FIG. 12D, 12E). Furthermore, in colons of Clec7a−/− mice the inventors detected increased message for TNF-α, IL-23p19 and IL-17a, cytokines previously associated with intestinal inflammation (Not shown). Without being bound to any particular theory, consistent with the hypothesis that altered immunity to gut fungi was responsible for the enhanced colitis, the inventors found that treatment with fluconazole (a specific anti-fungal drug) abrogated colitis in knockout animals, but had little or no effect for wild type animals. Further, the inventors found evidence of increased fungal burden in mucosa of DSS-treated knockout animals suggesting, without being bound to any particular theory, that in the absence of Dectin-1 the mice fail to contain commensal fungi when the mucosa is damaged by DSS. Without being bound to any particular theory the data demonstrate that Dectin-1 deficiency leads to increased susceptibility to colitis.

ASCA antibodies are directed against yeast mannan and have been successfully used as a marker of IBD in humans. However, it is unclear how these antibodies arise. The inventors measured ASCA antibody production in the serum of WT and Dectin-1 knockout mice after the onset of colitis to see if intestinal inflammation in mice causes the appearance of these antibodies and whether this might be regulated by Dectin-1. The inventors detected elevated amount of ASCA IgM and IgG in the serum of WT mice which remained constant 2 weeks after DSS treatment (FIG. 13 and data not shown). Despite increased disease severity and greater fungal burden, ASCA antibodies were not detected in the serum of Dectin-1 knockout mice. (FIG. 13). In contrast, antibody production against the bacterial antigen flagellin (FliC), which is often found associated with intestinal inflammation, was not affected by the absence of Dectin-1 (not shown). Thus, without being bound to any particular theory, the results suggest a specific role for Dectin-1 in the generation of effective antibody responses against intestinal fungal antigens.

The inventors have discovered a strong new association between genetic variants of Dectin-1 and IBD in humans. Based on the fungal distribution, where it is highest in the colon, the inventors used a mouse model of colitis, the inventors focused on ulcerative colitis (UC). The inventors focused on severe UC, termed medically refractory UC (MRUC). MRUC is defined as patients requiring colectomy as a result of lack of response to medication. The MRUC group was compared to a group of patients with UC who had not required colectomy (non-MRUC). The inventors identified an association of CLEC7A SNP r52078178 in patients with MRUC (logistic regression p=0.007). This association was significantly strengthened when combinations of CLEC7A SNPs were examined. A r52078178-r516910631 haplotype was strongly associated with MRUC (AG haplotype; p=0.00027; FIG. 14A), shorter time to surgery (p=0.0004) and thus with a more severe UC (FIG. 14B). When simply compared to non-diseased control subjects, the presence of the risk haplotype is not associated with developing ulcerative colitis. Without being bound to any particular theory, the inventors interpret the data to suggest that Dectin-1 (CLEC7A) is a gene involved in disease severity, not in disease susceptibility. Having the risk haplotype predisposes people to more severe disease should they develop ulcerative colitis due to other risk factors. Haplotypes were formed from rs2078178 and rs16910631 using PHASE v2.3. Haplotypes listed as “Other Combinations” were those that could not be reliably determined (posterior p<0.95).

TABLE 3
CLEC7A haplotypes associate with medically
refractive ulcerative colitis MRUC)

| | | Frequency | |
|---|---|---|---|
| Haplotype | | MRUC | Non-MRUC |
| rs2078178 | rs16910631 | (n = 323) | (n = 493) |
| G | G | 0.45 | 0.54 |
| G | G | | |
| G | G | 0.36 | 0.28 |
| A | G | | |
| A | G | 0.092 | 0.045 |
| A | G | | |

| | | |
|---|---|---|
| Other Combinations | 0.10 | 0.14 |

In addition to Dectin-1, genetic polymorphisms in CARD9 are also associated with IBD in humans. Without being bound to any particular theory, the inventors' studies and data suggest that the gut is home to commensal fungi that interact with our mucosal immune system in part through Dectin-1. Without being bound to any particular theory, this data suggests that the effectiveness of Dectin-1 immunity to commensal fungi may be an important variable in determining the severity of Ulcerative Colitis.

### Methods

Defining the fungal mycobiome. The inventors adapted and developed methodology for comprehensive analysis of fungal microbial communities in the gut using high throughput multitag sequencing of fungal rDNA.

Sequencing of highly variable regions of ribosomal DNA has become a standard method for identifying microbial species. Specific regions of ribosomal genes containing structural parts of ribosomal RNAs are highly conserved across species, making it possible to design generic primers to amplify ribosomal DNA from broad classes of related organisms (such as “bacteria” or “fungi”). Sequencing the highly variable internal parts of these amplified fragments permits accurate identification of the organisms. The intestinal bacterial microbiome has been frequently assessed by this method. For example, one recent application of this method identified over 5600 types of bacteria in the human gut. The method had not been applied to the mouse intestinal fungal microbiome, until this study. A small number of mostly environmental fungal communities had been previously analyzed using Roche 454 pyrosequencing, an established technology that provides relatively long reads (^{≈} 3-400 bases). The inventors have focused on moving the approach to Illumina Nex-gen sequencing, a newer technology that provides orders of magnitude more data, but in the form of shorter reads (^{≈}100-150 bases).

The inventors collected fecal specimens from the DSS-treated wild-type and Dectin-1 knockout mice, as well as human fecal specimens and amplified the ITS-2 (internal transcribed spacer-2) region of fungal ribosomal DNA. This region is now perhaps the most widely sequenced DNA region in fungi. Over 80,000 fungal ITS-2 sequences are currently found in Genbank representing over 2000 different fungal genera. It is becoming more popular than other rDNA regions (i.e., 18S) for microbial identification because it exhibits a higher degree of variation than other genic regions of rDNA. A recent study amplifying ITS-2 sequences from human oral cavities identified via Roche 454 pyrosequencing over 80 different fungi representing over 30 genera. The inventors subjected all of the DNA to Illumina Nex-gen sequencing in-house at the Cedars-Sinai Genetics Core, and subjected a subset of the samples to Roche 454 pyrosequencing at the UCLA Genomics Core in order to compare the two sequencing approaches. Samples from intestinal mucosal washes or biopsies are homogenized, and DNA is prepared. ITS-2 rDNA are amplified and prepared using differently barcoded primers for each sample. The samples are pooled for mass sequencing and later computationally resolved into individual samples for analysis. Since the amplified ITS-2 products contain sequences in proportion to the representation of each organism in the microbiome, the method allows for semi-quantitative representation of the relative amounts of each organism found in the sample. The inventors obtained over 750 Mb of Illumina sequence and over 30 Mb of 454 sequences containing over 6,000,000 and 400,000 individual sequences respectively.

The inventors have installed and extensively customized the QIIME pipeline for performing microbial community analysis on our local high performance computer cluster. QIIME is an open-source project developed in the Rob Knight Lab at the University of Colorado at Boulder that integrates many third party tools which have become standard in the field (qiime.sourceforge.net/index.html). The inventors have additionally built and curated a custom fungal sequence database assembled from sequences recovered from Genbank together with small database built for the “Fungal ITS Pipeline”. The current database the inventors tested includes over 100,000 fungal ITS sequences.

Bioinformatic methods for identification of Fungal species: Taxonomic and/or species assignment of bacteria based on 16s ribosomal RNA gene sequencing has been widely applied in recent studies. These approaches typically group sequence reads by similarity into Operational Taxonomic Units (OTU). Each OTU is then compared to a reference database using BLAST or similar sequence alignment algorithms to determine the species with the closest matching known sequence. This OTU-based analysis has many practical benefits; one of the biggest advantages is the reduction in required computational resources or analysis time. While some debate exists over the validity of this approach for the taxonomic assignment of DNA sequences from bacteria, there is a general consensus that it is sufficient for most clinical and research applications. Applying similar methods to the identification of fungal species is problematic. The range of sequence divergence both between and within species of fungi may differ <3% in ITS-2 sequence, approaching the error rate of the sequencing methods and making the delineation of sequence reads into OTUs less precise.

In order to avoid the spurious overlap of similar species which would skew the results, each unique sequence read generated in this study was queried against a custom-developed reference database. In addition, to the default parameters used, the inventors reduced redundancy in the data by removing duplicate reads. The approach is still far less computationally efficient than OTU-based methods (° 10K sequences processed per hour), but it circumvents the problematic steps of OTU assignment. Additionally, the inventors calculated the complete mapping for each read as identical bases aligned divided by the read length which helps improve species assignment compared to the OTU-based approach which does not account for incomplete mapping when assigning OTUs (data not shown). The majority of our sequences aligned to at least one sequence in the database with a complete mapping percentage ≥98%. These data correspond well to the 98% mapping cut-off has been previously used in the analysis of the fungal mycobiome.

From the sequence pool derived from mouse fecal specimens, a total of 324 unique fungal reference sequences had at least one read aligned to it, including 84 unnamed cultured species. Without being bound to any particular theory, this first look at the intestinal fungal mycobiome in mice reveals some interesting observations:

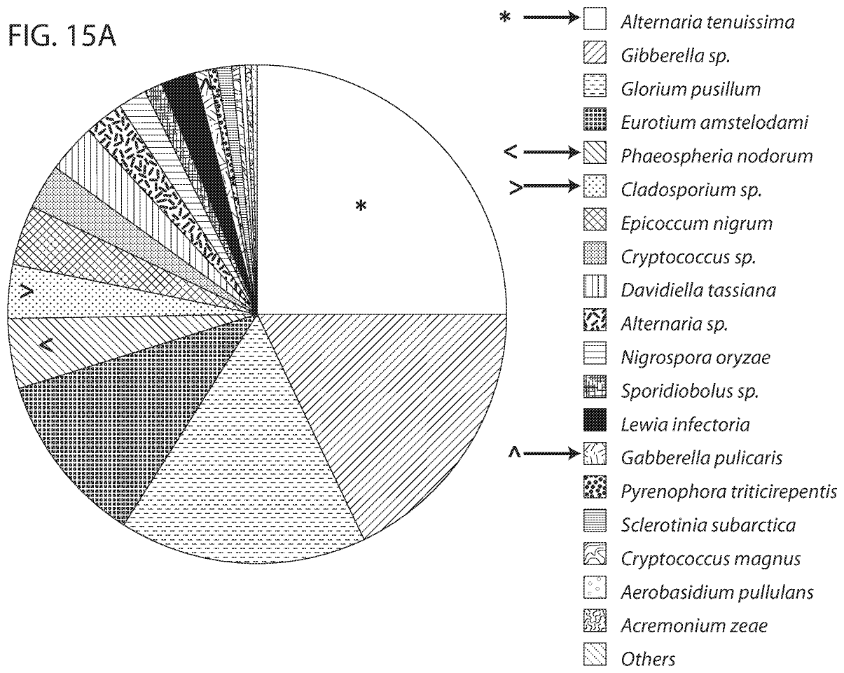

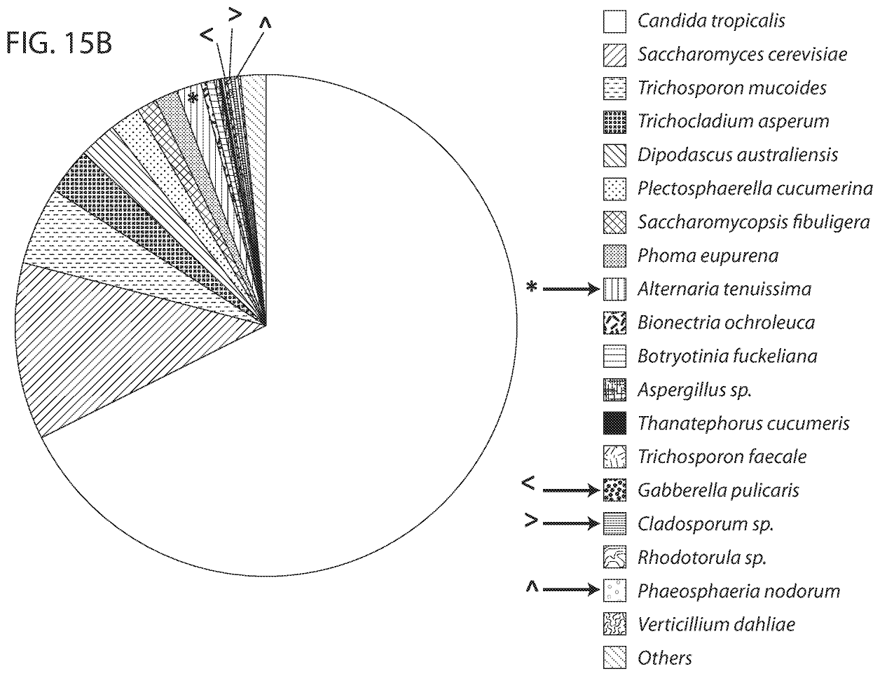

First, Candida tropicalis is the most prevalent commensal fungal species in the mice (FIG. 15). Candida sp. are known as gastrointestinal commensal organisms in humans and mice.

Second, the inventors also sequenced fungi from the mouse food and determined that fungi carried in food represent less than 2% of the fungal species detected in fecal specimens indicating that contamination with food-derived fungi is not a significant problem. This dataset will be an important reference for further studies.

Third, when broken down by DSS treated and untreated mice, many fungi change appreciably, a finding that was confirmed by quantitative PCR from a large sample of independent mice (not shown).

Fourth, principle component analysis suggests differences in the makeup of the fungal microflora in wild type and Dectin-1 knockout mice and differences arising after treatment with DSS (data not shown).

Fifth, the 454 and Illumina sequencing methods result in highly comparable fungal mycobiome definition, although the longer 454 sequencing reads generally pinned down species identifications, while the shorter Illumina reads reliably identify genus names but are less robust at identifying species names.

The inventors continue to characterize and explore the mouse intestinal fungal microbiome. Using strategies for monocolonization the inventors have found specific species that exacerbate colitis in mice as well as species that ameliorate disease. The inventors explore how the fungal microbiome interacts with the bacterial microbiome in mice, how it is affected by different genotypes, and how it behaves in other models of intestinal inflammation. This study is aimed at understanding how human genotype and disease interacts with the fungal microbiome.

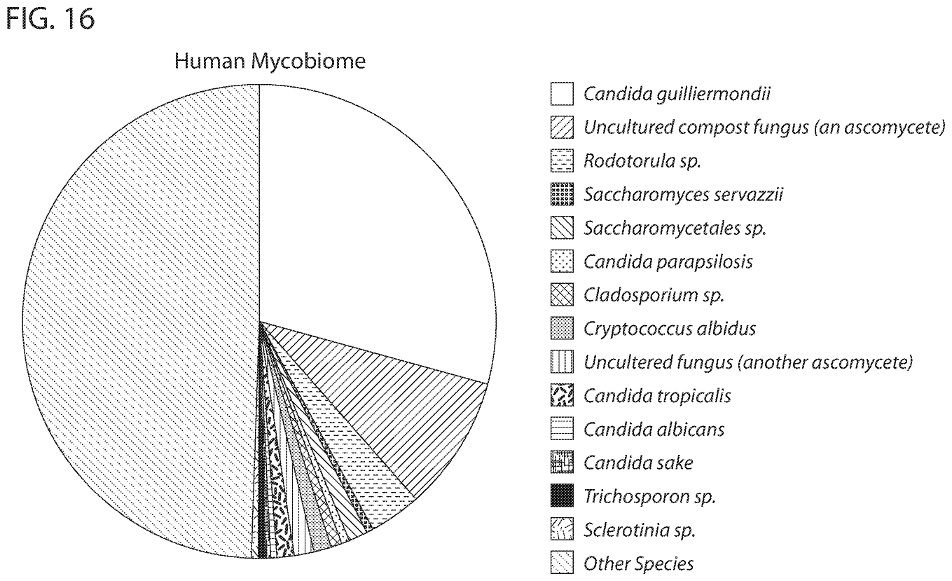

The inventors' preliminary data demonstrate that the human fungal microbiome is, like the mouse, highly diverse. In a “proof of principle” experiment, the inventors sequenced fungal ITS2 regions from two non-diseased human control fecal samples using the Illumina platform and characterized the major fungal constituents (FIG. 16). The inventors analyzed over 1.5 million sequences. S. cerevisiae was the dominant fungus, and identified over 400 separate species. Naturally, there is more variability between these two pilot patient samples than is typically seen between laboratory mice.

### Patient Sample Collection.

The inventors define the fungal mycobiome in mucosal washes & biopsies from IBD patients with and without the Dectin-1 risk polymorphism. The inventors collect colon biopsies and mucosal washes from IBD patients in the IBD center at Cedars-Sinai Medical Center. A subset of these (discussed below) will be subjected to fungal ITS sequencing, and the remainder will be archived for further analysis of specific fungal species (by quantitative PCR) and further fungal ITS2 and bacterial rDNA sequencing.

Male and female patients seen at Cedars Sinai Medical Center with a previous or suspected diagnosis of ulcerative colitis who are planned to undergo endoscopic evaluation of the colon are assessed for eligibility in this study as case subjects. Patients undergoing screening colonoscopy in a Cedars Sinai endoscopy unit are assessed for eligibility in this study as control subjects. All subjects are (or have been previously) consented under the IBD Center's Research Data Repository IRB Protocol. The data analysis for this study falls within the parameters of the original consent provided by the participants at the time of enrollment. Colonoscopy or flexible sigmoidoscopy are performed as a standard of care. Biopsies are routinely taken at the time of the procedure for diagnostic proposes. Additional biopsies and mucosal washings are collected as allowed under the IBD Center's IRB protocol. Study biopsies are collected from the sigmoid colon at approximately 20 cm proximal to the anal verge. The majority of study biopsies are snap frozen in liquid nitrogen and stored at −80° C. One biopsy is stored in RNA later for confirmatory quantitative real-time polymerase chain reaction (qPCR) analysis of microbiome analyses. One biopsy is paraffin-embedded for immunohistochemical analysis. In order to assess for significant differences in diet that may affect fungal analyses, subjects are asked to complete the Harvard Food Frequency Questionnaire at the time of specimen collection.

The inventors have full clinical information on the patients including a variety of measures of disease severity (hospitalization, blood tests, age of onset, endoscopy, etc.) as well as disease activity Mayo Scores at the time of collection. The inventors also have full genotyping data on these patients as they are typically in-house patients used in the ongoing GWAS studies at Cedars-Sinai (See A and B). (green is right column, blue is middle column, red is left column).

A) Enrollment Estimates:
Over 300 colonoscopies each year
Roughly 40% of these are UC patients
7% of these are homozygous for CLEC7A risk allele. (364 out of
5195 patients currently in our patient cohort).
Can expect to collect biopsies and mucosal washes from ≥15
homozygous risk allele patients/year.

| | Risk Allele | | Current Patient Database Totals |
|---|---|---|---|
| B) | CLEC7A | rs2078178-A/ | 364/2805/2026 |
| | | rs16910631-G | |
| | CARD9 | rs4077515-A | 1039/2512/1662 |
| | | rs10870077-C | 987/2494/1714 |

C) Exclusion Criteria:
Antibiotic treatment within last 3 months
Anti-fungal therapy within last 3 months
Major comorbidity or intercurrent illness
Vegetarian or therapeutic diet Immunomodulator or
immunosuppressant use in the previous 3 months
Green(right) = # Negative,
Blue(middle) = # Heterozygous,
Red(left) = # Homozygous

Given that around 7% of patients are homozygous for the CLEC7A risk allele, the inventors had no trouble collecting samples from around 45 of these patients over the 3-year period of the proposal (excluding those excluded from the study, see C above). The inventors collected a similar number of samples from more common patients homozygous for the protective allele for a total of 180 samples (biopsies+washes). The inventors subjected 10 of these samples each year to Fungal ITS sequencing to identify fungal communities (focusing first on the mucosal washes from UC patients with or without the CLEC7A risk allele, for a total of 30 samples for sequencing). The inventors continued to collect and analyze further samples to build a bigger database of fungal mycobiome (and associated bacterial biome) data to analyze in conjunction with SNP genotyping data in IBD patients.

The inventors explored whether alterations in the fungal mycobiome are associated with disease seventy and/or Dectin-1 haplotype. Specific changes noted in the sequencing data are further explored directly through quantitative real time PCR in additional samples not initially used for sequencing. The inventors explore whether variations in the bacterial microbiome in these samples are associated with Dectin-1 haplotype and/or variations in the fungal mycobiome. Without being bound to any particular theory, the inventors expected that since these microbial communities occupy the same niche there will be strong relationships between them.

Secondarily, the inventors make use of the larger database of genetic information about these patients to explore whether genetic variations in additional genes are associate with alterations in the fungal mycobiome. For example, the patient groups included patients with risk and protective alleles of two different CARD9 variants previously observed to be associated with IBD. This data is a rich characterization of an entirely novel gut microflora, a valuable dataset for further analysis, and promotes the further development of methodology for analysis of fungal microbiomes.

Additionally, the inventors searched for functional alterations in Dectin-1 that are associated with severe UC. The genetic data indicate that at least one variation in the Dectin-1 gene is associated with severe UC. The two SNPs that define the risk haplotype are both intronic. They could be functionally responsible for alteration in Dectin-1 expression, but they might also simply be in linkage disequilibnum with another variant that is even more strongly associated with severe UC and that is responsible for an alteration in Dectin-1 expression or function.

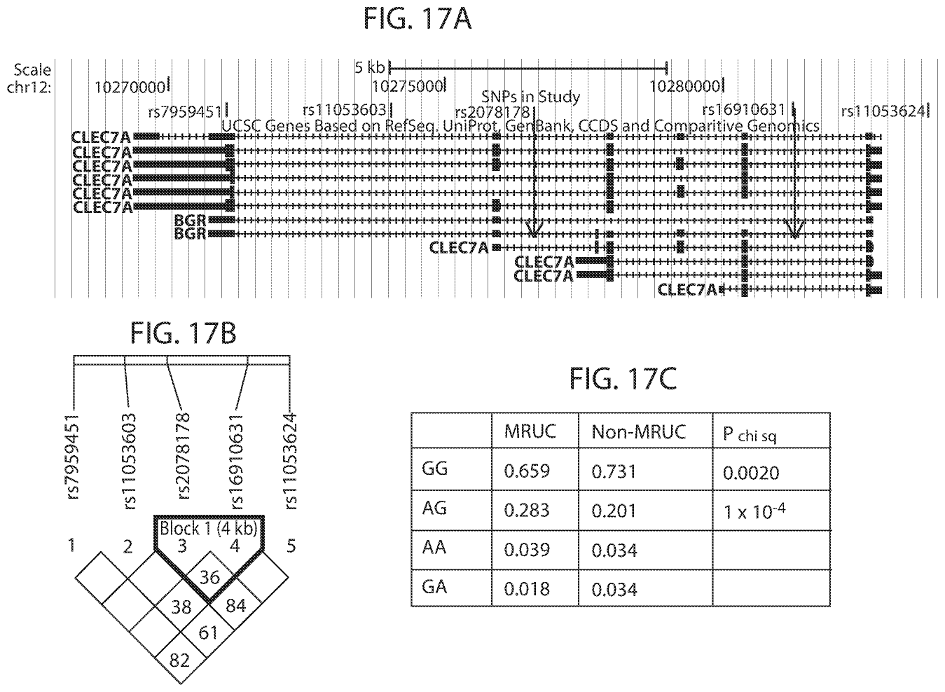

The two SNPs defining the Dectin-1 risk haplotype were discovered among the SNPs on the Illumina 370K chip used in the GWAS study from which the data were analyzed (FIG. 17A). These SNPs define a haplotype block (FIG. 17B) that is associated with severe UC (FIG. 17C). The inventors are working to refine the CLEC7A association by incorporating more than the 5 SNPs thus far evaluated. The inventors analyzed the entire UC patient set used in the initial analysis with a new custom “exome chip” that includes 38 CLEC7A SNPs. With these data (along with incorporating GWAS and Immunochip datasets) the inventors are able to narrow down the set of UC patients most affected by CLEC7A genotype.

Using these data, the inventors take two complementary approaches. First, the inventors sequence the gene (˜15 kb) from 20 patients carrying the most definitive risk allele and from 20 control patients. The inventors will search the sequence for variations that could be predicted to alter Dectin-1 expression or function and Dectin-1 expression and function are assessed. The inventors generated PBMCs, and assessed Dectin-1 expression and function.

Functional Analysis Methodology: The inventors identified individuals homozygous for the risk/protective polymorphisms in question and obtain blood from donors of the identified genotypes and isolate primary monocytes by a combination of ficoll plaque gradient centrifugation and monocyte specific magnetic bead isolation (Miltenyl). In order to evaluate as many cell types as possible for donor blood, the inventors differentiated a portion of the monocytes into macrophages and dendritic cells by culturing with recombinant M-CSF or GM-CSF/IL-4. The inventors assess Dectin-1 expression by flow cytometry commercially available antibodies that were used previously. For comparison, the inventors assessed surface expression of receptors that are not expected to be associated with CLEC7A genotype including FcRIII, CD11b, CD11c, CD14, and CD68. Further, the inventors performed immunoblots. Cell lysates were collected and accumulated, and protein expression levels were compared to controls in groups of 10 with comparison to tubulin as a loading control. This approach was previously undertaken by the inventors to determine that a CARD9 SNP associated with IBD is associated with overexpression of the protein in macrophages. The inventors additionally examined Dectin-1 expression by realtime PCR. Expression levels were normalized to EF1α.

In addition to expression level, the inventors exposed the cells from genotyped donors to a series of stimuli activating Fc receptors (IgG-coated microtiter plates & IgG-opsonized latex beads, MOI 5:1), Dectin-1 (glucan particles, 30 μg/ml, Biothera) and TLR4 (LPS, 100 ng/ml, Sigma), TLR2 (PAM3CSK4, 100 ng/ml, Invivogen) for 6 and 24 hours and measure production of multiple cytokines by Luminex bead assay. Cytokines to be measured included TNF-α, IL-1β, IL-6, IL-8, and GM-CSF (a Luminex set). In addition, the inventors measured production of a control cytokine not thought to be regulated by the NF-kB pathway (IFN-β). After stimulation, nuclear preps are collected, and NF-kB activation is measured by EMSA. Without being bound to any particular theory, these assays establish whether protein expression is affected by genotype and whether inflammatory signaling is affected by genotype.

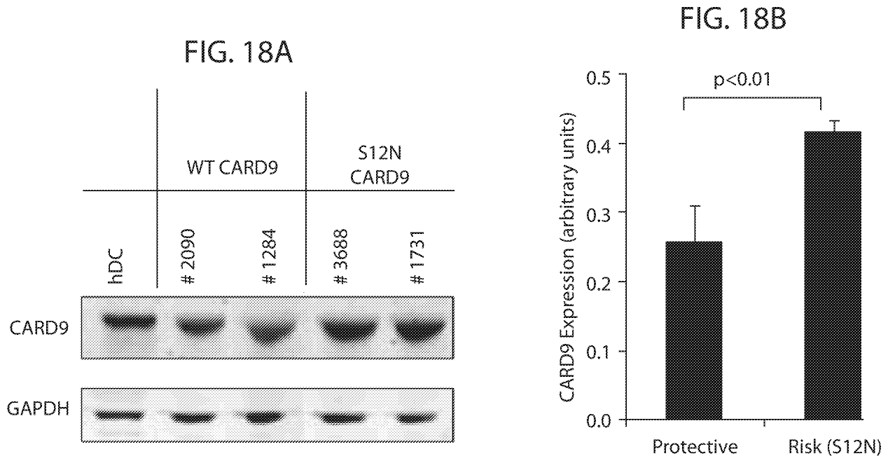

The inventors have assessed patients carrying protective and risk CARD9 alleles and observed that PBMCs from patients with the risk allele overexpress CARD9 (FIG. 18) and respond more vigorously to stimuli that activate CARD9 (not shown). The data in FIG. 18 also demonstrate the inventors' ability to identify key genotypes in the patient population, obtain blood samples from genotypes of interest, and assess expression and function.

In the second approach the inventors collected blood from patients carrying the risk allele or not, identified patients with the hypothesized alteration in Dectin-1 expression or function, and then sequenced the gene from these patients.

The inventors identify a coding region change that appears to be associated with loss or gain of function. Previous studies by the inventors demonstrate an in vitro reconstitution system in which specific variants of Dectin-1 (together with CARD9 or variants of CARD9) are ectopically expressed and expression and function assessed.

The inventors studied the role of fungi and alterations in anti-fungal immunity in genetically characterized ulcerative colitis patients. The gut microflora profoundly influenced intestinal inflammation through interacting with the immune system and activating specific inflammatory responses. Little is known about gut fungi and whether they too play a role in defining inflammatory responses at mucosal surfaces. Without being bound to any particular theory, the inventors suspect that alterations in how the immune system handles fungi found on colonic mucosal surfaces, affects severity of disease in certain ulcerative colitis patients. This understanding could lead to applying anti-fungal therapy to genetically-identified at-risk patients.

Without being bound to any particular theory, the findings suggest:
1) That the presence of Malassezia or Cladosporium are risk factors for disease and that screening for the presence of these organisms in colonoscopy samples or fecal samples can identify patients with a particular type of disease, and one that can respond to anti-fungal therapy.
2) That host interactions with fungi may be particularly dysregulated in individuals with specific CARD9 genotypes and that screening patients for CARD9 genotype can identify patients with a particular type of disease, and one that can respond to anti-fungal therapy.
3) That selection of an anti-fungal agent for use in treating IBD patients should be directed at choosing one that is effective against Malassezia (and/or Cladosporium) and that is available at high concentrations in the lumen of the colon.
4) That screening of patients for serum antibody reactivity to Malassezia (and/or Cladosporium) can be identify patients with a particular type of disease, and one that can respond to anti-fungal therapy. Serum test for Malassezia antigen is also possible.

Additional Sequences

| SEQ | |
|---|---|
| ID | |
| NO | Nucleotide Sequence |
| 1 | GGTGGGGGCA GGAGCCGGCA TCGCTCTCAG CACTGCCCCA |
| | CACTCCCCAG CCTGCCTGCT GAGGCCATGT CGGACTACGA |
| | GAACGATGAC GAGTGCTGGA Y CGTCCTGGAG GGCTTCCGGG |
| | TGACGCTCAC CTCGGTCATC GACCCCTCAC GCATCACACC |
| | TTACCTGCGG CAGTGCAAGG TCCTGAACCC CGATGATGAG |
| 2 | ATAGATAGAT AGGTGATAGA TGATAGACAG ATGGATACAT |
| | GGATAGATGA CAGGTAAGTC TGTAAGGTGG AGCGGGAAGG |
| | TTGAACACGG TTTTCCCTGA Y AGTAGGCTGG GGTGGGGAAG |
| | TATTGACTTC ATCATCGTTT ACTTTTTTTT AGTGTTAAGT |
| | ATTGCTTCTG TCATTTAAAA AACAGACCAG GTGTGGCAGC |
| 3 | CTCTGAGAAG TACTTCTTAA CGAAAGTAGC GTCAATTTAT |
| | TTCTTCAATT GGAGGAGAAG ACAATGGCTA ATCATCCATG |
| | AAAACTGCCT AGGGGGACTG Y CATCTGGGAT TTTGTCAGAT |
| | AGGTTGAATG GAGGCAGTAC AGTCCTCACA TTACAAATCT |
| | ATTCTTTTGG AAAATTAAAT GATAATTACT TTAAATAAAT |
| 4 | TTGTTGCATG ACCCTGGGCA AATTACTCCC TTTGTTGTTT |
| | TTTATTCCTC TTTGTGAAAC TGGGGTAATA ATATCTCAAA |
| | GGATTATTGC GGGAATTAAA Y GAGCTAACAC ATATAAAAGC |
| | ACCTTGTTTA GAGACTGCTA GCACATGATA GGTGTGTTGT |
| | TGCAGGATAC CGTTTGCCTA CACATATGCA TAAACATGCG |
| 5 | ATTTTCTGCA TTTATCTTGA CCTCAGCTGT TACTCTTTTC |
| | TGTTCTGTTT TCTGTCCTCC TTACTACCTC ACATATTTCT |
| | CTCTCCTTCT CCACCCTTCC Y CTTACATTGA |
| | AAACTTCTTC TCACAAATAC TATATGAGGG CACACTACAC |
| | AGTTGGTCAT AAATGACTGA CACGTGAATC CATACACAAT |
| | TTGGAGATGG |
| 6 | AAGGGGAAAA AAAGTAACAA TTCTAAATCT TTATGTGCCT |
| | GATAACACGG CTTCAACACA TCGAATAAAA TGTTTACAGA |
| | ACTGTAAGTA GAATCGCAGC Y GAAGATTTCA |
| | ACACAATTCT TTTCATATGG AAAGAGGAAG AAGCACGTAA |
| | AAATCACAGA ACTACAAGCA CGTGGAGCAT GTTCTCTGAT |
| | CACAGTGGAA |
| 7 | ACAGCTTATG GAAGGTGTTG GGACTGAAAT ATTATGTTAG |
| | ATGTTTGATT TTTTTTCCCT CTCTTCCAGT TTCCATAGTA |
| | ATGATCAAAT CAAAGAAATA Y AGAAAACACC |
| | TAACATTGCT GGAAACTGAA GAGGTGTTCA ACTCATTTAT |
| | CCGACCTTGA AGCCAGTAGA GTGCAGGAGA CATCTGAGTG |
| | TTGTCAAGGA |
| 8 | TACGAGAACGATGACGAGTGCTGGA[A/G/T]CGTCCTGGAG |
| | GGCTTCCGGGTGACG |
| 9 | GAAGGTTGAACACGGTTTTCCCTGA[C/G]AGTAGGCTGGGG |
| | TGGGGAAGTATTG |
| 10 | CCATGAAAACTGCCTAGGGGGACTG[C/T]CATCTGGGATTT |
| | TGTCAGATAGGTT |
| 11 | TCAAAGGATTATTGCGGGAATTAAA[A/C/G/T]GAGCTAAC |
| | ACATATAAAAGCACCTT |
| 12 | TTTCTCTCTCCTTCTCCACCCTTCC[C/T]CTTACATTGAAA |
| | ACTTCTTCTCACA |
| 13 | ACAGAACTGTAAGTAGAATCGCAGC[A/G]GAAGATTTCAAC |
| | ACAATTCTTTTCA |
| 14 | TAGTAATGATCAAATCAAAGAAATA[C/T]AGAAAACACCTA |
| | ACATTGCTGGAAA |
| 15 | TACGAGAACGATGACGAGTGCTGGAACGTCCTGGAGGGCTTC |
| | CGGGTGACG |
| 16 | GAAGGTTGAACACGGTTTTCCCTGACAGTAGGCTGGGGTGGG |
| | GAAGTATTG |
| 17 | CCATGAAAACTGCCTAGGGGGACTG[G/A]CATCTGGGATTT |
| | TGTCAGATAGGTT |
| 18 | TCAAAGGATTATTGCGGGAATTAAAGGAGCTAACACATATAA |
| | AAGCACCTT |
| 19 | TTTCTCTCTCCTTCTCCACCCTTCCNCTTACATTGAAAACTT |
| | CTTCTCACA |
| 20 | ACAGAACTGTAAGTAGAATCGCAGCNGAAGATTTCAACACAA |
| | TTCTTTTCA |
| 21 | TAGTAATGATCAAATCAAAGAAATANAGAAAACACCTAACAT |
| | TGCTGGAAA |

### Example 3

Described herein, we assess the ability of genetically defined risk factor to identify patients who might benefit from therapeutic manipulation of the intestinal microbiota. We investigate pharmacologically targeting fungi in the gut. Specifically, we are looking at a yeast commonly found on the skin but only recently found to be associated with the colonic mucosa in CD patients. Antibody markers including anti-fungal antibodies (e.g., ASCA) are useful in defining subsets of IBD patients, but the underlying mechanisms leading to antibody appearance are not always clear. We investigate the novel presence of fungal-specific antibodies in parallel with investigation of the microbiome and fungal-specific T cell responses.

We observed a strong association of Malassezia spp. (M. restricta & M. globosa) with CD. Malassezia spp. are known to be major fungal commensal colonizers of the skin and are involved in pathology of some skin conditions, but that they might be important in the gut is unexpected. Innate inflammatory responses to fungi are mediated in large part through a signaling pathway involving a protein called CARD9. GWAS studies linked a polymorphism in CARD9 (S12N) to the risk of CD, and we found that the presence of Malassezia was positively linked to the presence of the CARD9 risk allele. We further found that the disease-associated risk allele of CARD9 specifically enhances human inflammatory responses to Malassezia and that Malassezia exacerbates colitis via CARD9 in mouse models of disease.

Without wishing to be bound by any particular theory, we believe that oral antifungal treatment reduces the burden of Malassezia spp. in CD patients with the CARD9 S12N allele. Further, we believe that the microbial changes induced by antifungal treatment are associated with dampened downstream immune responses in those with a genetic predisposition to developing strong immune responses to Malassezia.

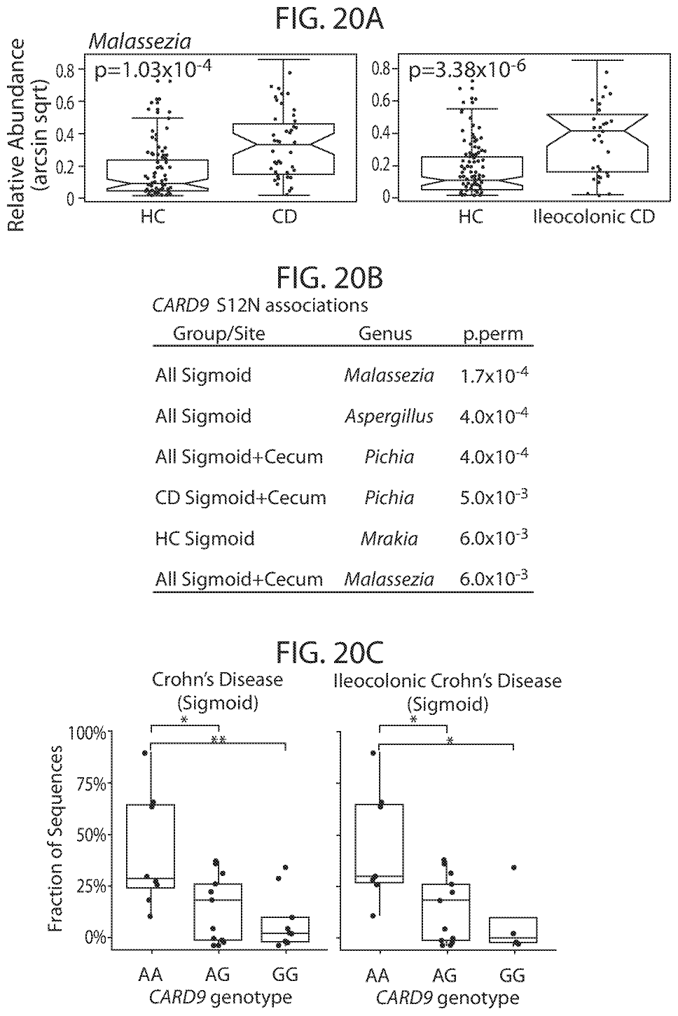

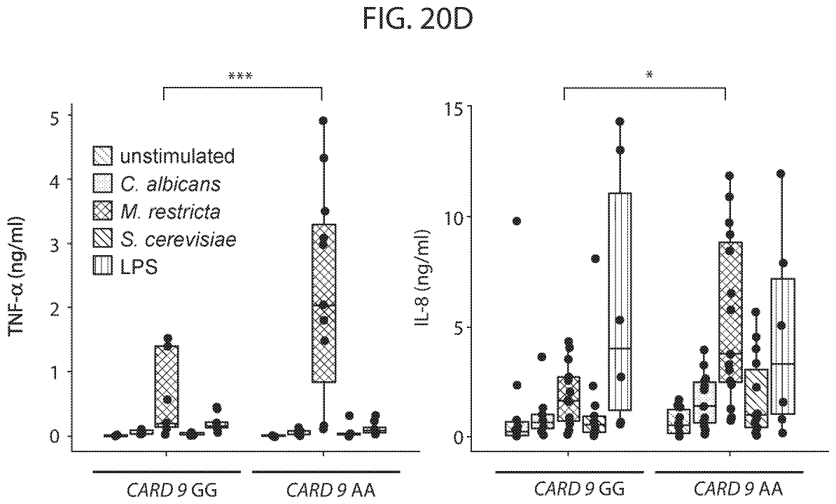

Based on the above-mentioned association between anti-Saccharomyces cerevisiae antibodies (ASCA) and increasing interest in the role of the microbiome in IBD, we embarked on a multi-year study to determine if alteration in the intestinal fungal microbiota are associated with subsets of patients with CD. While most intestinal microbiome studies rely on fecal samples, we reasoned that samples taken directly from the colonic mucosa might be more enriched in organisms that interact directly with the immune system and influence tissue inflammatory responses. We analyzed over 160 mucosal washing samples collected during colonoscopy of patients with Crohn's disease and healthy controls at Cedars-Sinai Medical Center (CSMC) by high throughput sequencing of fungal rDNA “internal transcribed spacer 1” (ITS1) genes. We observed a strong association of Malassezia spp. (M. restricta & M. globosa) with CD (FIG. 20A).

Malassezia spp. are members of the Basidiomycota phyla of fungi and, while all other members of the Ustilaginomycotina subdivision are plant pathogens, Malassezia are commensal skin microbes found on nearly all warm-blooded animals. Malassezia have among the smallest of eukaryotic genomes, having only around 4000 genes. They grow mainly as yeasts, although some species can develop hyphae. An important feature of Malassezia genomes is the loss of key enzymes required for lipid metabolism, including fatty acid synthase (FAS), A desaturase, and A enoyl CoA isomerase. Therefore, they cannot produce fatty acids themselves and need lipids from the environment for growth. In the skin, they harvest lipids from sebum in hair follicles through secretion of a host of lipases and phospholipases. These enzymes can release unsaturated free fatty acids from sebum lipids including oleic acid and arachidonic acid that can be inflammatory. That Malassezia spp. might be important in intestinal inflammation is unexpected.

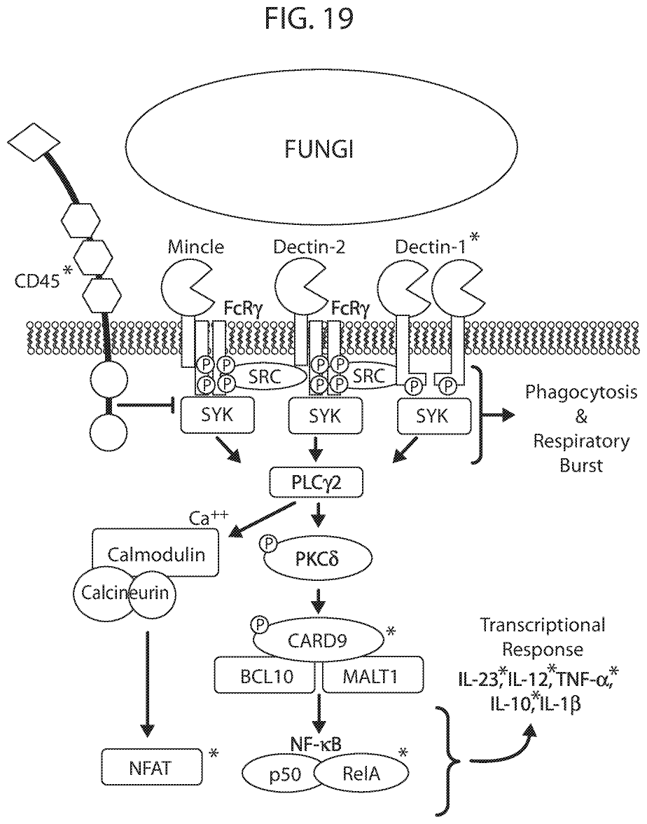

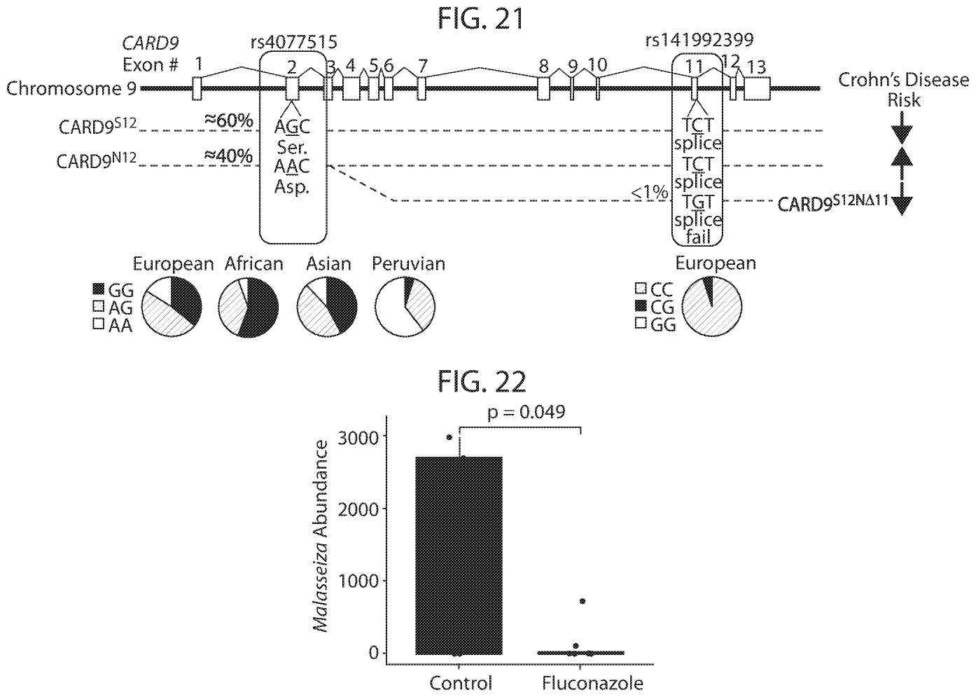

Innate immune responses to fungi are mediated in large part by the C-type lectin family of receptors that includes Dectin-1, Dectin-2 and Mincle. These receptors trigger inflammatory response upon detecting fungi through a signaling pathway that uses a protein called CARD9 to activate NF-κB (FIG. 19). GWAS studies have linked a nonsynonymous single nucleotide polymorphism (SNP) in exon 2 of CARD9 to the risk of CD. This SNP leads to production of a CARD9^{S12N} variant of the protein. The allele frequency of the “minor” N12 coding variant is around 40% and present broadly in all ethnic groups examined (FIG. 21). It is slightly less frequent in Africans and slightly more frequent in native South American populations. In Europeans, a rare second polymorphism at the end of exon 11 linked to the CARD9^{N12} variant results in production of a splice variant lacking exon 11 and production of a protein lacking function. While the N12 variant confers increased risk of developing Crohn's Disease, the truncation variant is protective. The CARD9^{S12N} polymorphism has also been linked to the risk of developing ulcerative colitis, primary sclerosing cholangitis, ankylosing spondylitis, and immunoglobulin A (IgA) nephropathy. The high prevalence of the gene variant suggests that it may be protective against certain diseases, most likely specific fungal pathogens, but this has not yet been proven.

We investigated whether this polymorphism was linked to the presence of specific fungi. Among patients with CD, the presence of Malassezia was positively linked to the presence of the CARD9 risk allele (FIG. 20B-C). To investigate whether the CARD9 risk allele altered how these patients respond to fungi, we collected blood from healthy volunteers, homozygous for the CARD9 risk allele or protective allele (via the “Material and Information Resources for Inflammatory and Digestive Diseases” (MIRIAD) biobank at CSMC, the largest repository of IBD specimens in the world). We stimulated monocyte-derived dendritic cells with Malassezia and other yeasts and found that the risk allele made the cells respond more vigorously (FIG. 20D).

To test whether Malassezia could influence intestinal inflammation, we used DSS and T cell transfer models of colitis in mice and found that Malassezia potently exacerbated disease. To evaluate whether oral antifungals can directly affect Malassezia levels in the gut, we treated mice with oral fluconazole and measured depletion of Malassezia from the stool (FIG. 22).

Together the data indicate that association of Malassezia with the intestinal mucosa may exacerbate disease in a subset of patients who can be identified by their genetics and/or serological markers.

Acquisition of Samples after Antifungal Treatment in Genetically Defined Patients with Active Crohn's Disease.

Details of trial design. Described herein we investigate our belief that CD patients with the CARD9^{S12N} allele may benefit from therapy targeting suppression of intestinal Malassezia. This trial will test the antimicrobial and anti-inflammatory effects of oral posaconazole in patients with active Crohn's disease. Enrolled patients will be selected to be homozygous for the CARD9^{S12N} risk allele from previously genotyped patients in the Cedars-Sinai MIRIAD registry. This will be single-site trial enrolling adults with endoscopically active Crohn's disease. Subjects will be required to have failed conventional therapy with corticosteroids or immunomodulators; previous biologic exposure will be permitted. Concomitant conventional and biologic CD therapy at stable doses will be permitted.

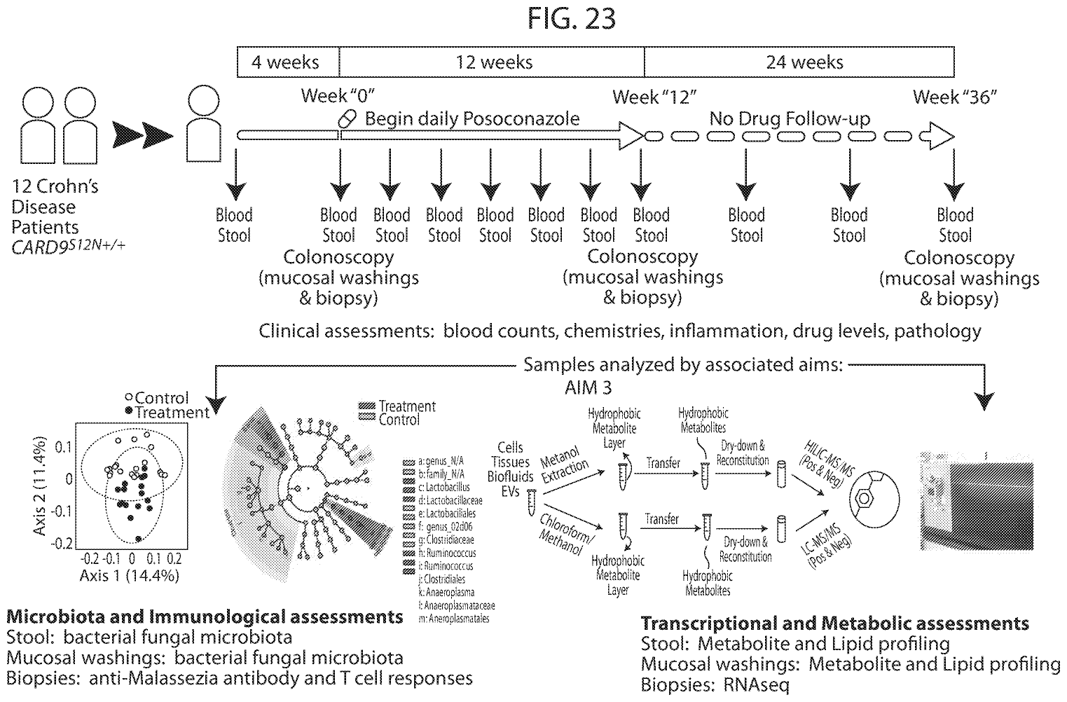

Posaconazole is an azole antifungal agent selected due to its low MIC against most Malassezia spp., its high concentration in feces with low first pass effect that will help optimize topical distal bowel exposure, and the ability to measure its systemic concentration in blood. Subjects will be treated for 12 weeks at an FDA-approved dose; treatment duration with posaconazole for invasive fungal infections frequently exceed 3 months, thus providing confidence in the safety of the dose and duration proposed for the current study. After subjects are consented and enrolled, baseline laboratory, stool, and endoscopic evaluations will be performed during a 4-week screening period (FIG. 23). Endoscopic evaluations will be recorded, and mucosal washings and biopsies collected from involved and uninvolved segments. 15 subjects will be treated with oral posaconazole (Noxafil®), 300 mg twice daily for the first day followed by 300 mg daily for 12 weeks. Blood and stool samples will be collected to assess blood counts, chemistries, inflammation and posaconazole levels every 2 weeks in order to monitor clinical changes and assess safety. At 12 weeks, subjects will undergo clinical and endoscopic assessment (including mucosal washings and biopsies) to assess treatment response and will discontinue posaconazole. Subjects will then be followed for an additional 24 weeks, during which subjects will undergo monthly clinical, biochemical, and microbiomal evaluations, followed by a final colonoscopy with sample collections at the end of the 24 week period. Samples collected during the follow-up period will be used to assess safety, to determine the durability of stool microbiome changes, and to assess sustainability of any clinical and endoscopic improvement. Serum, stool, and colonoscopic washings and biopsies obtained during clinical visits and procedures will be used as described herein. The primary endpoint is endoscopic improvement in Crohn's disease activity, defined by a 50% decrease in the Simple Endoscopic Score for Crohn's Disease (SES-CD) at week 12. Secondary endpoints include clinical remission (CDAI<150 points), normalization of inflammatory markers, and improvement in health-related quality of life.

Enrollment. This will be a single-center open-label study. Patients will be recruited from the Cedars-Sinai Inflammatory Bowel Disease Center, which has an experienced clinical trials team that has participated in and successfully recruited patients for more than 20 clinical trials in IBD over the past 10 years. Approximately 240 adult outpatients with Crohn's disease are seen monthly by 7 IBD physicians and 2 nurse practitioners. Based on current genetic analyses of this population, we estimate that ≈25% of these patients are homozygous for the risk CARD9 allele, and that ≈20% will be on stable therapy with active disease. Thus, approximately 12 eligible patients would be seen monthly, and if only 10%-20% of eligible patients enroll we conservatively estimate an enrollment pace of 1-2 subjects per month, and a total enrollment period of up to 12 months.

Statistical methods. The primary endpoint is endoscopic improvement, defined as a 50% reduction SES-CD scores at week 12. Key secondary endpoints will include clinical remission at week 12, defined by a Crohn's disease Activity Index <150. Other secondary endpoints will include normalization of inflammatory biomarkers, and improvement in health-related quality of life. Assuming a drop-off rate of 15% before the second colonoscopy and alpha=0.05 and 80% power, we estimate that a sample size of n=15 will be necessary to determine an expected 30% improvement from an expected endoscopic improvement rate of 13% with no therapy.

Monitoring for adverse effects (AE): Prior to initiating therapy, subjects will be screened for electrolyte abnormalities and concomitant medications that may be affected by posaconazole metabolism (CYP3A4). Subjects will undergo a baseline a baseline and end-of-study EKG to assess for QT prolongation. AEs will be assessed at every study visit. Common AE associated with posaconazole in clinical trials included diarrhea, dyspepsia, nausea, fever, headache, coughing, and hypokalemia (from package insert). Adverse events will be monitored at each study visit and reported per local and federal guidelines. AEs will be reviewed by a Data Safety Monitoring Board after 6 subjects have completed 12 weeks of treatment. The DSMB will be comprised of a statistician, gastroenterologist, and a clinical trial expert. Excessive toxicity stopping rules: Rule 1: If at any point during the trial the probability of developing Grade 3 AE is above 10%, posaconazole therapy will be deemed unsafe and the trial will stop accrual. If 4 or more patients with Grade 3 AE are observed the trial will be terminated. The probability of observing 4 or more Grade 3 AEs and incorrectly terminating the trial is 0.0256. This is equivalent to a one-sided binomial test with a sample size of 15 patients, 2.5% significance and 80% power where the null hypothesis is p=0.1 and the alternative is p>0.41. Rule 2: If there is an indication that the probability of developing Grade 4 AE is above 2.5%, posaconazole therapy will be deemed unsafe and the trial will stop accrual. If 2 or more patients with Grade 4 AE are observed the trial will be terminated. The probability of observing 2 or more Grade 4 AEs and incorrectly terminating the trial is 0.0349. This is equivalent to a one-sided binomial test with a sample size of 15 patients, 3.5% significance and 80% power were the null hypothesis is p=0.025 and the alternative is p>0.23.

### Effects of Treatment on the IBD Myco/Microbiome.

We focus on understanding the use of oral antifungal drugs to target Malassezia populations in the intestinal gut mucosa in CD patients. While our primary goal is to assess changes in Malassezia populations and anti-Malassezia immune reactivity, it is also important to understand more broadly the effects of oral antifungals on the gut. We investigate the antifungal therapy's impact on the fungal and bacterial mucosa-associated microbiota in patients, as well as intestinal metabolites. We will further investigate how well microbiota changes noted in stool reflect microbiota changes observe in the human intestinal mucosa. Finally, we will assess immune responses to Malassezia and their changes before and after treatment.

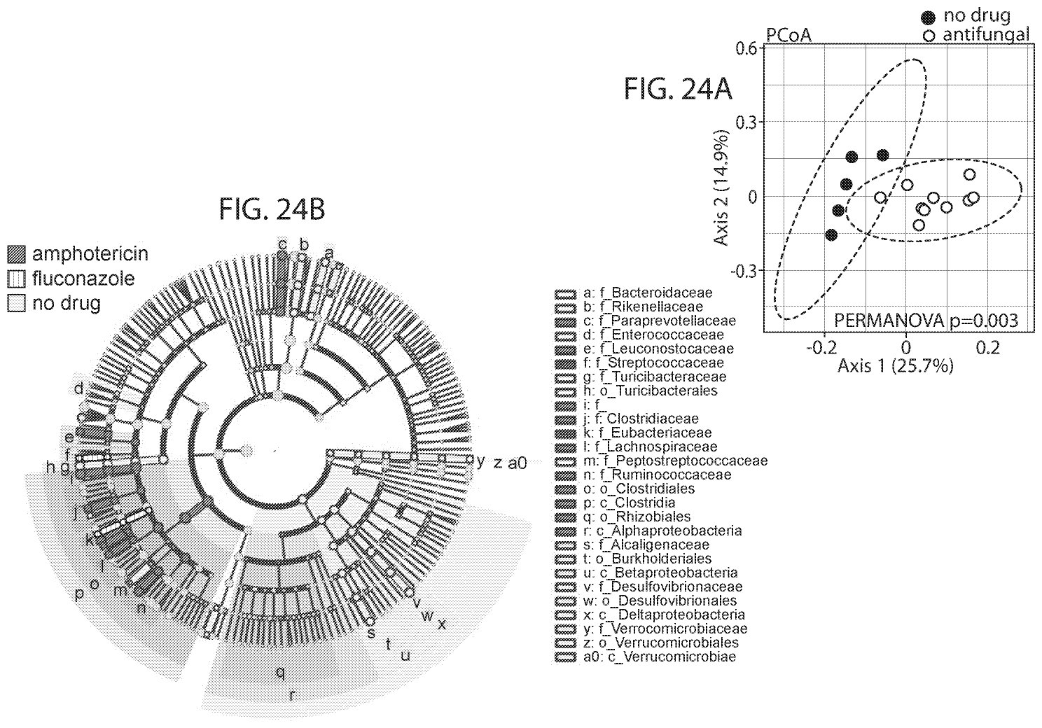

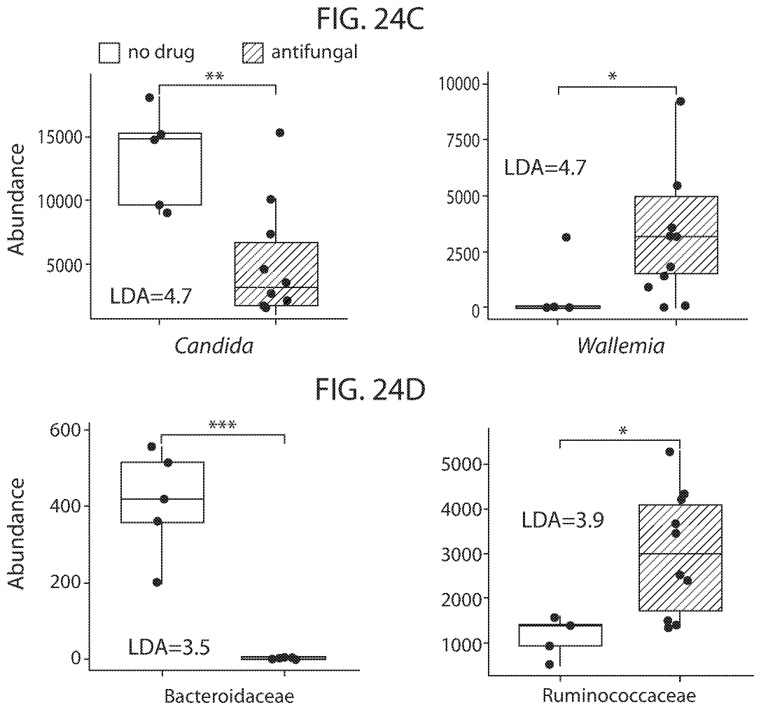

We will investigate the impact of antifungal therapy on the mucosa-associated microbiota in patients by examining samples collected before and after anti-fungal therapy. We will investigate the association between the mycobiota detected by high-throughput ITS (fungal) and 16S (bacterial) rDNA sequencing as well as targeted quantitative PCR in colonic washes, biopsies, and fecal samples. In preliminary studies in mice, we have observed that oral treatment with antifungal drugs (we have tried several) alters the fungal microbiota in fecal samples, which is, of course, not surprising (FIGS. 24A&C), but it also affects the bacterial microbiota (FIGS. 24B&D). The interrelationship between bacterial and fungal microbiota and the susceptibility of these communities to manipulation by antifungal drugs in mice is being further studied in other funded work. In this project we will investigate the effects of antifungal drug treatment on the bacterial and fungal microbiota in people. While not wishing to be bound by any particular theory, we believe that we will observe similar effects on the fungal and bacterial microbiota in patients receiving oral antifungal therapy.

We will prepare DNA from the mucosal washing, stool samples, and biopsies using methods we have developed specifically to optimally recover DNA from fungi. We will analyze bacterial and fungal 16S and ITS1 content by high-throughput sequencing (Illumina MiSeq platform with paired end 300 bp read) as illustrated above. For sequence analyses raw FASTQ data will be filtered to enrich for high quality reads including removing the adapter sequence by cutadapt v1.4.1, demultiplexing and truncating reads not having an average quality score of 20 (Q20) over a 3 base pair sliding window, and removing any reads that do not contain the proximal primer sequence or any reads containing a single N (unknown base). Filtered pair-end reads will be merged with overlap into single reads using SeqPrep v1.0 wrapped by QIIME v1.6 with default settings. 16S OTU assignment will be made by alignment with the GreenGenes reference database at 97% identity. ITS1 sequences will be first aligned to previously-observed host sequences (including rRNA, olfactory receptor and uncharacterized genes in human) to deplete potential contamination, then operational taxonomic unit (OTU) will be picked by aligning filtered reads to the in-house Targeted Host Fungi (THF) custom fungal ITS database (currently version 1.6), using BLAST v2.2.22 in the QIIME v1.6 wrapper with an identity percentage ≥97%. OTUs with average relative abundance >0.00001 will be considered to be present as well as compiled into genera for downstream analysis.

Alpha diversity measures of OTUs will be calculated including Shannon's diversity index, inverse Simpson index, observed OTUs with R package phyloseq v1.16.2 on the unfiltered OTU table. Filtered OTU tables will be used for all other downstream analysis. Two-sided Wilcoxon test will be used to examine differences in alpha diversity indices. Pair-wise differential abundance analysis will be conducted using linear discriminant analysis with LEfSe v 1.38.0 at default settings as well as negative binomial Wald test with phyloseq (FDR corrected p value <0.05), respectively. A multivariate association analysis will be conducted by using MaAsLin v0.0.3 with minimum OTU prevalence of 0.05, minimum abundance of 0.001 and FDR-corrected p-value cutoff of 0.10, on the variables of interest: drug, time, age, gender, site of disease.

Statistical considerations: In our human mycobiome studies detecting a difference in Malassezia levels between Crohn's Patients having the CARD9 AA allele and the GG allele we observed Malassezia proportions in our sigmoid colon sequencing data of 20%+14% and 5%+5% respectively. Assuming a similar change might be induced in CARD9 AA patients upon treatment with antifungal antibiotics and, based on these numbers and an alpha 0.5/90% power, we will need a sample size of at least 9 to detect a difference in our patients. PCR approaches to detecting Malassezia to employed in aim 1 should be even more precise.

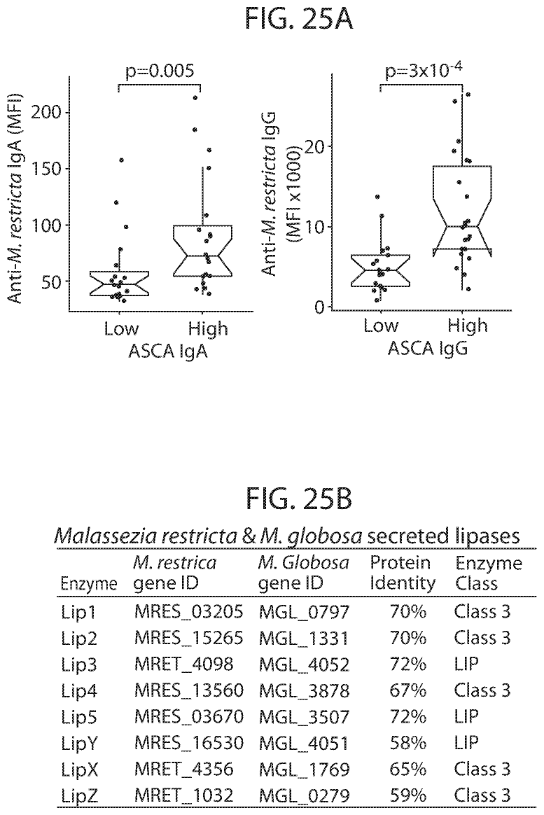

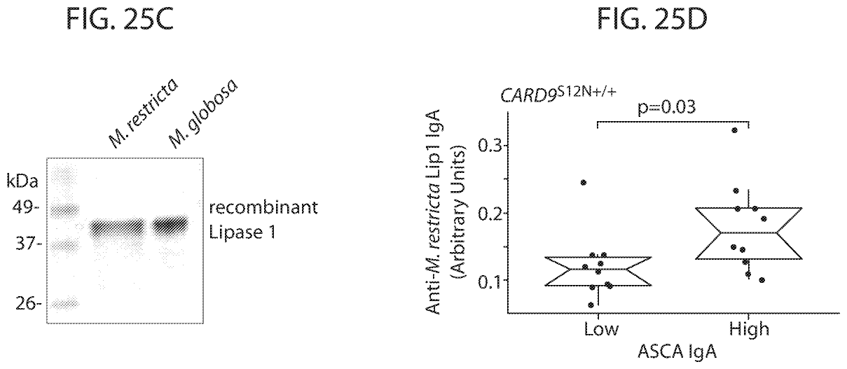

We will investigate human seroreactivity against Malassezia spp. (Specifically, Malassezia restricta and Malassezia globosa). As noted above, ASCA, antibodies recognizing yeast mannans, are increased in patients with IBD. To investigate whether ASCA antibodies might be reactive against Malassezia, we measured binding of IgA and IgG to M. restricta in serum from Crohn's disease patients characterized as being “Low” or “High” in ASCA. In both cases we observed that ASCA sera were reactive with M. restricta (FIG. 25A). Reactivity of ASCA with Malassezia, however, does not tell us which patients might have a specific immune response to Malassezia.

As discussed above, Malassezia cannot produce fatty acids themselves but need lipids from the environment for growth. To harvest lipids from their environment, they secrete a host of highly active lipases that are characteristic of Malassezia (FIG. 25B). We hypothesized that these enzymes might act as Malassezia-specific antigens during host interactions with the fungi. We therefore cloned and recombinantly produced M. restricta and M. globosa lipase 1 (Lip1, FIG. 25C). We found that sera from CD patients with low ASCA levels responded poorly to M. globosa and M. restricta Lip1 (not shown), while ASCA+sera revealed anti-Malassezia Lip1-specific IgA when the patients were homozygous for the CARD9^{S12N} (FIG. 25D). We observed similar results when assessing anti-Lip1 IgG and Lip1 from M. globosa. These data are consistent with the hypothesis that CD in CARD9^{S12N} patients is characterized by unusual exposure and immune response to Malassezia.

While not wishing to be bound by any particular theory, we believe that Malassezia-specific antibody production may be a clinically useful tool in assessing the nature of a patient's disease. We will recombinantly produce each of the Malassezia lipases in FIG. 25B as well as Candida albicans lipase 1 (same enzyme class, but not homologous to Malassezia Lip1) for comparison. We develop ELISA plate-based assays for IgG and IgA reactivity against the proteins. We obtain anti-sera from patients and healthy controls characterized as high or low for ASCA and homozygous for the CARD9^{S12} or CARD9^{N12} alleles from the MIRIAD Biobank. We compare seroreactivity against Malassezia lipases with genotype and ASCA levels (as in FIG. 25D) as well as with antibody reactivity to bacterial antigens (OmpC, fliC) and to nuclear antigens (pANCA). These later data are already part of the MIRIAD

Biobank Data Associated with the Stored Sera.

Statistical considerations: Based on the variance in our preliminary data and an alpha=0.05, a group size of at least 11 will confer 80% power (www.stat.ubc.ca/˜rollin/stats/ssize/n2.html). Since these analyses will be performed on banked patient sera and the genotypes to be tested are not rare, sample availability will not be a barrier. We will evaluate antigen reactivity in at least 20 sera of each genotype. Data will be evaluated by ANOVA with Tukey's multiple comparison test where necessary. Mann-Whitney U Test will be performed where data fail typical tests for normality (i.e. as seen in FIG. 25).

We will test the hypothesis that CARD9^{S12N} CD patients will preferentially produce Th17 CD4⁺ T cells specific for Malassezia. Bacher et al. recently observed that a high fraction (>10%) of CD154⁺ CD4⁺ T cells from healthy donors produce IL-17 and IL-22 when restimulated with Candida albicans, while there is little response to Malassezia. These findings were interpreted to suggest that commensal gut fungi (Candida) are an important source of naturally-occurring Th17 cells and that these cells are an important factor in mucosal immunity. We anticipate that CD patients exposed to Malassezia in the gut mucosa will develop a strong Th17 CD4 T cell response to Malassezia and that this may serve as an independent test of immunological encounter with Malassezia in this population of patients.

To test for development of Malassezia-specific T cell responses in patients, we will use the approach developed by Bacher et al. to evaluate T cell responses to fungi in healthy volunteers. Specifically, we will compare Malassezia-reactive Th17 CD4+ T cell responses in healthy controls (n=10 each, homozygous for CARD9S12 or CARD9N12) and Crohn's disease patients of both genotypes. Fresh blood from genotyped control donors will be provided by the MIRIAD biobank, as we have previously done. CD patient blood will be provided by subjects examined in Aim1 as well as the MIRIAD biobank. The potential for a specific role of Malassezia-induced Th17 cells in causing or driving disease is not implied, and further studies directly examining the functional consequence of this population of cells would be appropriate for future studies.

Statistical considerations: Based on the variance data in Bacher et al. and an alpha=0.05, a group size of at least 6 will confer 80% power. Data will be evaluated by ANOVA with Tukey's multiple comparison test where necessary. Mann-Whitney U Test will be performed should data fail typical tests for normality.

Expected outcomes and alternative approaches: We will have a more complete understanding of the effect of oral antifungal drug on the intestinal bacterial microbiota. We anticipate that posaconazole will alter the bacterial, as well as the fungal, microbiota and that both changes will be reflected in metabolite profiles. We anticipate that bacterial and fungal communities may revert to their pre-drug structures upon cessation of treatment. It is possible that recovery will not be complete or will revert to “new” states. In a recent study on humans taking antibiotics, the bacterial microbiota largely normalized 21 days after ceasing treatment.

We anticipate no problems in making the recombinant proteins or evaluating seroreactivity as illustrated in the preliminary data (2B). Lip1 already looks like a good candidate for assessing Malassezia-specific antibody responses, but we will determine whether one of the other lipases might offer stronger or more consistent signals. Without wishing to be bound by any particular theory, we believe that anti-Malassezia immune responses will characterize a specific subset of CD patients (ASCA+CARD9^{S12N}) and that this signal will not correlate with other serum markers. We routinely get fresh blood from genotyped CD patients in our IBD center and anticipate no problems collecting sufficient samples for the T cell analyses (2C).

### Effects of Treatment on the Transcriptional and Metabolic Signatures of Inflammation.

This study focuses on understanding the use of oral antifungal drugs to target Malassezia populations in the intestinal gut mucosa in CD patients, and while our primary goal is to assess changes in Malassezia populations and anti-Malassezia immune reactivity, it is also important to understand more broadly the effects of oral antifungals on the gut. Described above, we examined the effects of anti-fungal treatment on bacterial and fungal populations overall. We also explore the effects of oral antifungal treatment on the spectrum of metabolites found in stool and endoscopy samples as well as transcriptional activity in the gut mucosa of CD patients.

We investigate the impact of antifungal therapy on the mucosa-associated and fecal metabolome in patients by examining samples collected before and after anti-fungal therapy. We evaluate the presence of metabolites in samples by reversed-phase LC-MS/MS. While not wishing to be bound by any particular theory, we believe that treatment with posaconazole consistently induce changes in specific metabolites. Such changes may have functional consequences and/or may serve as useful biomarkers of disease.

Based on these findings, we believe that it is likely that we will observe drug-induced alterations in stool and mucosal washing (mucosa-associated) metabolites in the patient samples collected. Mass spectrometry-based measurements of metabolites (the metabolome) will be performed with the Cedars-Sinai Proteomics and Metabolomics Core Facility. For LC-MS/MS evaluations, we extract hydrophilic metabolites from stool and mucosal washing samples with methanol and dry down the metabolite-containing upper layer under nitrogen. We extract hydrophobic metabolites with chloroform/methanol and dry down the metabolite-containing lower layer under nitrogen. Both samples will be reconstituted in appropriate buffers and subjected to reversed-phase LC-MS/MS with a positive or negative ion-scanning mode. A targeted MS-based analyses capable of measuring up to 219 polar small molecule metabolites will be employed. Metabolomic analyses are performed using a dedicated triple quadrupole LC/MS (Agilent) instrument capable of precise, reproducible, and quantitative metabolite analysis.

As discussed above, Malassezia are unusual among yeasts in that they require long chain fatty acids for growth and have thus adapted to produce multiple secreted lipases that they use to process and harvest lipids from their environment. We therefore have a special interest in whether posaconazole treatment alters the lipid profile of the mucosal/intestinal environment.

To specifically profile lipids in patient stool and mucosal washing samples, we extract lipids with methyl-tert-butyl ether and dry the sample and resuspend lipids in an isopropanol/methanol mixture for analysis together the Cedars-Sinai Proteomics and Metabolomics Core Facility. The core lipid analysis pipeline provides a profile encompassing 1153 lipid species from 13 different lipid classes including quantitative information of more than 500 different triacylglycerol (TAG) species, 26 cholesterol esters (CE), and many more species from various lipid classes. Data will be acquired using a Lipidyzer (Sciex), a QTRAP mass spectrometer equipped with Differential Mobility Spectrometry (DMS), which functions to separate isobaric lipid species, critical to the depth of the lipid assay and proper analyte specific quantitation. This assay has been used extensively by the Core on plasma, serum, cells, tissue and isolated exosomes. The standards used for quantitation are based on lipid classes, to which each analyte is specifically normalized.

We will investigate the impact of antifungal therapy on the intestinal transcriptome. To determine whether anti-fungal drug-induced changes in the microbiome/metabolome are also reflected in gene expression patterns in the host mucosa, we perform RNA-seq on biopsies collected before anti-fungal drug treatment, after treatment (12 weeks) or after recovery (24 weeks).

Samples are processed for RNA sequencing. Briefly, DNA & RNA will be isolated from RNA-later-preserved biopsies using the AllPrep DNA/RNA Universal Kit from Qiagen. Samples will be cut into 20-25-mg pieces on a dry ice bath and placed in tubes for bead-based mechanical homogenization. After homogenization, the lysate will be passed through an AllPrep DNA Mini spin column that allows selective and efficient binding of genomic DNA which will be eluted in TE buffer after on-column proteinase K digestion. Genomic DNA will be preserved for future reference and evaluation of microbial DNA. Flow-through from the spin column will be digested with proteinase K and passed over an RNeasy Mini spin column. RNA will be eluted in water after on-column DNaseI digestion. RNA library construction will be performed using the Illumina TruSeq Stranded mRNA library preparation kit (Illumina, San Diego, Calif.). cDNA will be synthesized from enriched and fragmented RNA using reverse transcriptase (Invitrogen, Carlsbad, Calif.) and random primers. The cDNA will be further converted into double-stranded DNA (dsDNA), and the resulting dsDNA enriched with PCR for library preparation. PCR-amplified library will be purified using Agencourt AMPure XP beads (Beckman Coulter, Brea, Calif.). Sample libraries will be multiplexed and sequenced on a NextSeq 500 platform (Illumina) using 75 bp single-end sequencing. On average, about 20 million reads are typically generated from each sample.

Raw reads obtained from RNA-Seq will be aligned to the transcriptome using STAR (version 2.5.0) (45)/RSEM (version 1.2.25) with default parameters, using a custom human GRCh38 transcriptome reference downloaded from www.gencodegenes.org, containing all protein coding and long non-coding RNA genes based on human GENCODE version 23 annotation. Expression counts for each gene (TPM: transcripts per million) in all samples will be normalized by the sequencing depth.

Expected outcomes and alternative approaches: Upon completion of this, we understand the effects of oral anti-fungal treatment on intestinal metabolite and transcriptional profiles. Based on our preclinical data, we expect to observe alterations in intestinal metabolites induced by posaconazole.

While preferred embodiments have been shown and described herein, it will be obvious to those skilled in the art that such embodiments are provided by way of example only. Numerous variations, changes, and substitutions will occur to those skilled in the art without departing from the scope of this application. Various alternatives to the embodiments described herein may be employed in practicing the scope of this application.

1. A method of treating an inflammatory disease or condition in a subject in need thereof, comprising:
administering an anti-fungal therapy to the subject in need thereof,
wherein the subject in need thereof has a fungus or an elevated level of a fungus as compared to a control level of the fungus in the gastrointestinal system.

2. A method of manipulating the microbiome of a subject having an inflammatory disease or condition, comprising:
administering an anti-fungal therapy to the subject,
wherein the subject in need thereof has a fungus or an elevated level of a fungus as compared to a control level of the fungus in the gastrointestinal system.

3. A method of inhibiting or reducing fungal growth in a subject having an inflammatory disease or condition, comprising:
administering an anti-fungal therapy to the subject in need thereof,
wherein the subject in need thereof has a fungus or an elevated level of a fungus as compared to a control level of the fungus in the gastrointestinal system.

4. The method of claim 1, wherein the subject in need thereof has a c-type lectin domain containing 7A (CLEC7A) gene risk variant, a caspase recruitment domain family member 9 (CARD9) gene risk variant or both.

5. The method of any of claim 1, wherein the subject in need thereof has a caspase recruitment domain family member 9 (CARD9) gene risk variant A in rs4077515 resulting in an amino acid substitution S12N.

6. The method of claim 1, wherein the subject has an elevated level of a serological marker, as compared to a control level of the serological marker, said serological marker selected from the group consisting of anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globosa antibody, anti-Cladosporium spp. antibody, and a combination thereof.

7. The method of claim 1, wherein the fungus is Malassezia, Cladosporium or both.

8. The method of claim 1, wherein the inflammatory disease is inflammatory bowel disease.

9. The method of claim 1, wherein the inflammatory disease is ulcerative colitis or Crohn's disease.

10. The method of claim 1, wherein the anti-fungal therapy comprises posaconazole.

11. The method of claim 1, wherein the anti-fungal therapy comprises a fungicide agent, a fungistatic agent, or an antimycotic agent.

12. (canceled)

13. The method of claim 11, wherein antimycotic agent comprises a polyene, an azole, an echinocandin, a flucytosine, an allylamine, a tolnaftate, or griseofulvin.

14. The method of claim 13,
wherein the azole comprises triazole, imidazole, clotrimazole, ketoconazole, itraconazole, terconazole, oxiconazole, miconazole, econazole, tioconazole, voriconazole, fluconazole, isavuconazole, itraconazole, pramiconazole, ravuconazole, or posaconazole,
wherein the polyene comprises amphotericin B, nystatin, or natamycin,
wherein the echinocandin comprises caspofungin, anidulafungin, or micafungin, and
wherein the allylamine comprises naftifine or terbinafine.

15. (canceled)

16. (canceled)

17. (canceled)

18. A method of determining a level of a fungus, a fungal antigen, an anti-fungal antibody, or an enzyme of a fungus, in a subject in need thereof, comprising
obtaining a biological sample from the subject;
subjecting the biological sample to an assay suitable to detect the fungus, the fungal antigen the anti-fungal antibody, or the enzyme of a fungus, in the biological sample; and
measuring the level of the fungus, the fungal antigen, the anti-fungal antibody, or the enzyme of the fungus, to determine the level of the fungus, the fungal antigen, the anti-fungal antibody, or the enzyme of the fungus.

19. A method of diagnosing an inflammatory disease or condition in a subject, comprising:
determining a level of a fungus, a fungal antigen, an anti-fungal antibody, or an enzyme of a fungus, in a subject in need thereof accordingly to claim 18; and
diagnosing the subject with the inflammatory disease or condition when an elevated level of the fungus, the fungal antigen, the anti-fungal antibody, or the enzyme of a fungus as compared to each respective control level, is measured in the biological sample.

20. The method of claim 18,
wherein detecting the presence of the fungal antigen comprises contacting an antibody capable specifically binding to the fungal antigen to the biological sample to form a binding complex, and detecting the presence of the binding complex,
wherein detecting the presence of anti-fungal antibody comprises contacting an antibody capable specifically binding to the anti-fungal antibody to form a binding complex, and detecting the presence of the binding complex,
wherein detecting the presence of the enzyme of a fungus comprise contacting an antibody capable specifically binding to the enzyme to form a binding complex, and detecting the presence of the binding complex.

21. (canceled)

22. (canceled)

23. The method of claim 18, wherein the biological sample is selected from the group consisting of blood plasma, blood serum, stool, intestinal aspirate, intestinal tissue sample, intestinal mucosa, intestinal mucus and combinations thereof.

24. The method of claim 18, wherein
the fungus is Malassezia, Cladosporium, or both,
the fungal antigen is from Malassezia, Cladosporium, or both,
the anti-fungal antibody is an antibody capable of specifically binding to Malassezia, Cladosporium, or both, or specifically binding to an antigen of Malassezia, Cladosporium, or both, and
the enzyme is a lipase.

25. The method of claim 18, wherein the enzyme is Malassezia lipase 1 or Cladosporium lipase 1.

26. The method of claim 18, wherein the subject in need thereof has a c-type lectin domain containing 7A (CLEC7A) gene risk variant, a caspase recruitment domain family member 9 (CARD9) gene risk variant or both.

27. The method of claim 18, wherein the subject in need thereof has a caspase recruitment domain family member 9 (CARD9) gene risk variant A in rs4077515 resulting in an amino acid substitution S12N.

28. The method of claim 18, wherein the subject has an elevated level of a serological marker, as compared to a control level of the serological marker, said serological marker selected from the group consisting of anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globosa antibody, anti-Cladosporium spp. antibody, and a combination thereof.

29. The method of claim 18, wherein the subject has an elevated level of a serological marker, as compared to a control level of the serological marker, said serological marker is an enzyme of a fungus.

30. The method of claim 29, wherein the enzyme is Malassezia lipase 1, or Cladosporium lipase 1.


SEQUENCE LISTING (29 sequences)

SEQ ID NO: 1 - DNA, 201 bp, Homo sapiens
ggtgggggcaggagccggcatcgctctcagcactgccccacactccccagcctgcctgctgaggccatgtcggactacgagaacgatgacgagtgctggaycgtcctggagggcttccgggtgacgctcacctcggtcatcgacccctcacgcatcacaccttacctgcggcagtgcaaggtcctgaaccccgatgatgag

SEQ ID NO: 2 - DNA, 201 bp, Homo sapiens
atagatagataggtgatagatgatagacagatggatacatggatagatgacaggtaagtctgtaaggtggagcgggaaggttgaacacggttttccctgayagtaggctggggtggggaagtattgacttcatcatcgtttacttttttttagtgttaagtattgcttctgtcatttaaaaaacagaccaggtgtggcagc

SEQ ID NO: 3 - DNA, 201 bp, Homo sapiens
ctctgagaagtacttcttaacgaaagtagcgtcaatttatttcttcaattggaggagaagacaatggctaatcatccatgaaaactgcctagggggactgycatctgggattttgtcagataggttgaatggaggcagtacagtcctcacattacaaatctattcttttggaaaattaaatgataattactttaaataaat

SEQ ID NO: 4 - DNA, 201 bp, Homo sapiens
ttgttgcatgaccctgggcaaattactccctttgttgttttttattcctctttgtgaaactggggtaataatatctcaaaggattattgcgggaattaaaygagctaacacatataaaagcaccttgtttagagactgctagcacatgataggtgtgttgttgcaggataccgtttgcctacacatatgcataaacatgcg

SEQ ID NO: 5 - DNA, 201 bp, Homo sapiens
attttctgcatttatcttgacctcagctgttactcttttctgttctgttttctgtcctccttactacctcacatatttctctctccttctccacccttccycttacattgaaaacttcttctcacaaatactatatgagggcacactacacagttggtcataaatgactgacacgtgaatccatacacaatttggagatgg

SEQ ID NO: 6 - DNA, 201 bp, Homo sapiens
aaggggaaaaaaagtaacaattctaaatctttatgtgcctgataacacggcttcaacacatcgaataaaatgtttacagaactgtaagtagaatcgcagcygaagatttcaacacaattcttttcatatggaaagaggaagaagcacgtaaaaatcacagaactacaagcacgtggagcatgttctctgatcacagtggaa

SEQ ID NO: 7 - DNA, 201 bp, Homo sapiens
acagcttatggaaggtgttgggactgaaatattatgttagatgtttgattttttttccctctcttccagtttccatagtaatgatcaaatcaaagaaatayagaaaacacctaacattgctggaaactgaagaggtgttcaactcatttatccgaccttgaagccagtagagtgcaggagacatctgagtgttgtcaagga

SEQ ID NO: 8 - DNA, 51 bp, Homo sapiens
  misc_feature (26)..(26): n is a, c, g, t or u
tacgagaacgatgacgagtgctggancgtcctggagggcttccgggtgacg

SEQ ID NO: 9 - DNA, 51 bp, Homo sapiens
  misc_feature (26)..(26): n is a, c, g, t or u
gaaggttgaacacggttttccctganagtaggctggggtggggaagtattg

SEQ ID NO: 10 - DNA, 51 bp, Homo sapiens
  misc_feature (26)..(26): n is a, c, g, t or u
ccatgaaaactgcctagggggactgncatctgggattttgtcagataggtt

SEQ ID NO: 11 - DNA, 51 bp, Homo sapiens
  misc_feature (26)..(26): n is a, c, g, t or u
tcaaaggattattgcgggaattaaangagctaacacatataaaagcacctt

SEQ ID NO: 12 - DNA, 51 bp, Homo sapiens
  misc_feature (26)..(26): n is a, c, g, t or u
tttctctctccttctccacccttccncttacattgaaaacttcttctcaca

SEQ ID NO: 13 - DNA, 51 bp, Homo sapiens
  misc_feature (26)..(26): n is a, c, g, t or u
acagaactgtaagtagaatcgcagcngaagatttcaacacaattcttttca

SEQ ID NO: 14 - DNA, 51 bp, Homo sapiens
  misc_feature (26)..(26): n is a, c, g, t or u
tagtaatgatcaaatcaaagaaatanagaaaacacctaacattgctggaaa

SEQ ID NO: 15 - DNA, 51 bp, Artificial Sequence
  synthetic construct
tacgagaacgatgacgagtgctggaacgtcctggagggcttccgggtgacg

SEQ ID NO: 16 - DNA, 51 bp, Artificial Sequence
  synthetic construct
gaaggttgaacacggttttccctgacagtaggctggggtggggaagtattg

SEQ ID NO: 17 - DNA, 51 bp, Artificial Sequence; identical to SEQ ID NO: 10
  synthetic construct
  misc_feature (26)..(26): n is a, c, g, t or u

SEQ ID NO: 18 - DNA, 51 bp, Artificial Sequence
  synthetic construct
tcaaaggattattgcgggaattaaaggagctaacacatataaaagcacctt

SEQ ID NO: 19 - DNA, 51 bp, Artificial Sequence; identical to SEQ ID NO: 12
  synthetic construct
  misc_feature (26)..(26): n is a, c, g, t or u

SEQ ID NO: 20 - DNA, 51 bp, Artificial Sequence; identical to SEQ ID NO: 13
  synthetic construct
  misc_feature (26)..(26): n is a, c, g, t or u

SEQ ID NO: 21 - DNA, 51 bp, Artificial Sequence; identical to SEQ ID NO: 14
  synthetic construct
  misc_feature (26)..(26): n is a, c, g, t or u

SEQ ID NO: 22 - DNA, 22 bp, Artificial Sequence
  synthetic construct
cttggtcatttagaggaagtaa

SEQ ID NO: 23 - DNA, 20 bp, Artificial Sequence
  synthetic construct
gctgcgttcttcatcgatgc

SEQ ID NO: 24 - DNA, 23 bp, Artificial Sequence
  synthetic construct
tctcatgcaccaccatcaaggac

SEQ ID NO: 25 - DNA, 23 bp, Artificial Sequence
  synthetic construct
tgaccactctccctttgcagaac

SEQ ID NO: 26 - DNA, 23 bp, Artificial Sequence
  synthetic construct
atccagttgccttcttgggactg

SEQ ID NO: 27 - DNA, 23 bp, Artificial Sequence
  synthetic construct
taagcctccgacttgtgaagtgg

SEQ ID NO: 28 - DNA, 20 bp, Artificial Sequence
  synthetic construct
ggctgtattcccctccatcg

SEQ ID NO: 29 - DNA, 22 bp, Artificial Sequence
  synthetic construct
ccagttggtaacaatgccatgt